SEQUENCE LISTING (172 sequences)

SEQ ID NO: 1 - protein, 5 aa, Artificial Sequence
  Synthetic
SYAIS

SEQ ID NO: 2 - protein, 17 aa, Artificial Sequence
  Synthetic
GIIPIFGTANYAQKFQG

SEQ ID NO: 3 - protein, 10 aa, Artificial Sequence
  Synthetic
GSGTLNWFDP

SEQ ID NO: 4 - protein, 101 aa, Artificial Sequence
  Synthetic
QVQLVQSGGTFSSYAISWVRQAPGQGLEWMGGIIPIFGTANYAQKFQGRVTITADESTSTAYMELSSLRSEDTAVYYCARGSGTLNWFDPWGQGTLVTVSS

SEQ ID NO: 5 - protein, 452 aa, Artificial Sequence
  Synthetic
METDTLLLWVLLLWVPGSTGQVQLVQSGGTFSSYAISWVRQAPGQGLEWMGGIIPIFGTANYAQKFQGRVTITADESTSTAYMELSSLRSEDTAVYYCARGSGTLNWFDPWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 14 aa, Artificial Sequence
  Synthetic
TGTSSDIGGYNFVS

SEQ ID NO: 7 - protein, 7 aa, Artificial Sequence
  Synthetic
EVSKRPS

SEQ ID NO: 8 - protein, 11 aa, Artificial Sequence
  Synthetic
SSNSGDYADVV

SEQ ID NO: 9 - protein, 111 aa, Artificial Sequence
  Synthetic
QSVLTQPRSVSGSPGQSVTISCTGTSSDIGGYNFVSWYQQHPGKAPKLVITEVSKRPSGVPDRFSGSKSGNTASLTISGLQAEDEADYYCSSNSGDYADVVFGGGTKLTVL

SEQ ID NO: 10 - protein, 238 aa, Artificial Sequence
  Synthetic
METDTLLLWVLLLWVPGSTGQSVLTQPRSVSGSPGQSVTISCTGTSSDIGGYNFVSWYQQHPGKAPKLVITEVSKRPSGVPDRFSGSKSGNTASLTISGLQAEDEADYYCSSNSGDYADVVFGGGTKLTVLGQPKANPTVTLFPPSSEELQANKATLVCLISDFYPGAVTVAWKADGSPVKAGVETTKPSKQSNNKYPRTSSYLSLTPEQWKSHRSYSCQVTHEGSTVEKTVAPTECS

SEQ ID NO: 11 - protein, 5 aa, Artificial Sequence
  Synthetic
GYYMH

SEQ ID NO: 12 - protein, 17 aa, Artificial Sequence
  Synthetic
RINPNSGGTNYAQKFQG

SEQ ID NO: 13 - protein, 11 aa, Artificial Sequence
  Synthetic
VTYNIGWYIDY

SEQ ID NO: 14 - protein, 120 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSS

SEQ ID NO: 15 - protein, 471 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 11 aa, Artificial Sequence
  Synthetic
GGNNIGSKGVH

SEQ ID NO: 17 - protein, 7 aa, Artificial Sequence
  Synthetic
YDTDRPS

SEQ ID NO: 18 - protein, 11 aa, Artificial Sequence
  Synthetic
QVWDSSSDHPV

SEQ ID NO: 19 - protein, 108 aa, Artificial Sequence
  Synthetic
QAVVTQPPSVSVAPGKTATITCGGNNIGSKGVHWYQQKPGQAPVLVIYYDTDRPSGIPERLSGSNSGNTATLTISRVEAGDEADYYCQVWDSSSDHPVFGGGTKLTVL

SEQ ID NO: 20 - protein, 235 aa, Artificial Sequence
  Synthetic
METDTLLLWVLLLWVPGSTGQAVVTQPPSVSVAPGKTATITCGGNNIGSKGVHWYQQKPGQAPVLVIYYDTDRPSGIPERLSGSNSGNTATLTISRVEAGDEADYYCQVWDSSSDHPVFGGGTKLTVLGQPKANPTVTLFPPSSEELQANKATLVCLISDFYPGAVTVAWKADGSPVKAGVETTKPSKQSNNKYPRTSSYLSLTPEQWKSHRSYSCQVTHEGSTVEKTVAPTECS

SEQ ID NO: 21 - protein, 17 aa, Artificial Sequence
  Synthetic
WINPNSGGTNYAQKFQG

SEQ ID NO: 22 - protein, 18 aa, Artificial Sequence
  Synthetic
GMWVPSITMIVGGGAFDI

SEQ ID NO: 23 - protein, 127 aa, Artificial Sequence
  Synthetic
EVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGMWVPSITMIVGGGAFDIWGQGTMVTVSS

SEQ ID NO: 24 - protein, 478 aa, Artificial Sequence
  Synthetic
METDTLLLWVLLLWVPGSTGEVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGMWVPSITMIVGGGAFDIWGQGTMVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 16 aa, Artificial Sequence
  Synthetic
RSSESLLHRNGFNYLD

SEQ ID NO: 26 - protein, 7 aa, Artificial Sequence
  Synthetic
MGSYRAS

SEQ ID NO: 27 - protein, 11 aa, Artificial Sequence
  Synthetic
MQALQSPPLYT

SEQ ID NO: 28 - protein, 114 aa, Artificial Sequence
  Synthetic
DVVMTQSPLSLPVTPGEPASMSCRSSESLLHRNGFNYLDWYVQRPGQSPKLLIYMGSYRASGVPDRFSGRGSGTDFALKISRVEAEDVGVYYCMQALQSPPLYTFGPGTKLEIK

SEQ ID NO: 29 - protein, 243 aa, Artificial Sequence
  Synthetic
METDTLLLWVLLLWVPGSTGDVVMTQSPLSLPVTPGEPASMSCRSSESLLHRNGFNYLDWYVQRPGQSPKLLIYMGSYRASGVPDRFSGRGSGTDFALKISRVEAEDVGVYYCMQALQSPPLYTFGPGTKLEIKRTVPRTPSAVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 5 aa, Artificial Sequence
  Synthetic
GYYWS

SEQ ID NO: 31 - protein, 16 aa, Artificial Sequence
  Synthetic
EINHSGSTNYNPSLKS

SEQ ID NO: 32 - protein, 8 aa, Artificial Sequence
  Synthetic
GWYLGFDY

SEQ ID NO: 33 - protein, 117 aa, Artificial Sequence
  Synthetic
QVQLQQWGAGLLKPSETLSLTCAVYGGSFSGYYWSWIRQPPGKGLEWIGEINHSGSTNYNPSLKSRVTISVDTSKNQFSLKLSSVTPRTDTAVYYCARGWYLGFDYWGQGTLVTVSS

SEQ ID NO: 34 - protein, 14 aa, Artificial Sequence
  Synthetic
TGTSRDVGGYDYVS

SEQ ID NO: 35 - protein, 7 aa, Artificial Sequence
  Synthetic
GVSERPS

SEQ ID NO: 36 - protein, 10 aa, Artificial Sequence
  Synthetic
CSYAGSFTWV

SEQ ID NO: 37 - protein, 110 aa, Artificial Sequence
  Synthetic
QSVLTQPRSVSGSPGQSVTISCTGTSRDVGGYDYVSWYQQYPGKAPKLMISGVSERPSGVPDRFTGSRSANTASLTISGLQTDDEANYYCCSYAGSFTWVFGDGTKLTVL

SEQ ID NO: 38 - protein, 7 aa, Artificial Sequence
  Synthetic
SSSYYWG

SEQ ID NO: 39 - protein, 16 aa, Artificial Sequence
  Synthetic
SIYYSGSTYYNPSLKS

SEQ ID NO: 40 - protein, 26 aa, Artificial Sequence
  Synthetic
QANQVVPPRTIPWAKPGGTPPNWFDP

SEQ ID NO: 41 - protein, 137 aa, Artificial Sequence
  Synthetic
QVQLQESGPGLVKPSETLSLTCTVSGGSISSSSYYWGWIRQPPGKGLEWIGSIYYSGSTYYNPSLKSRVTISVDTSKNQFSLKLSSVTPRTDTAVYYCARQANQVVPPRTIPWAKPGGTPPNWFDPWGQGTLVTVSS

SEQ ID NO: 42 - protein, 11 aa, Artificial Sequence
  Synthetic
GGNNIGSKSVH

SEQ ID NO: 43 - protein, 7 aa, Artificial Sequence
  Synthetic
YDSARPS

SEQ ID NO: 44 - protein, 12 aa, Artificial Sequence
  Synthetic
QVWDSSSDHPRV

SEQ ID NO: 45 - protein, 109 aa, Artificial Sequence
  Synthetic
QLVLTQPPSVSVAPGETARITCGGNNIGSKSVHWYQQKPGQAPVLVIYYDSARPSGIPERVSGSNSGNTATLTISRVEAGDEADYYCQVWDSSSDHPRVFGTGTKLTVL

SEQ ID NO: 46 - protein, 5 aa, Artificial Sequence
  Synthetic
DYAVS

SEQ ID NO: 47 - protein, 20 aa, Artificial Sequence
  Synthetic
FIESKPYGETREYPRTSVRG

SEQ ID NO: 48 - protein, 7 aa, Artificial Sequence
  Synthetic
VVAWVAY

SEQ ID NO: 49 - protein, 119 aa, Artificial Sequence
  Synthetic
QVQLVESGGDLVNPGRSLRLACTGAGFTFGDYAVSWFRQAPGKGLEWVGFIESKPYGETREYPRTSVRGRFTISRDDSKGIAYLQMNGLKTEDTGVYYCSSVVAWVAYWGQGTLVTVSS

SEQ ID NO: 50 - protein, 11 aa, Artificial Sequence
  Synthetic
RASQSVTTYLN

SEQ ID NO: 51 - protein, 7 aa, Artificial Sequence
  Synthetic
GTSALQS

SEQ ID NO: 52 - protein, 9 aa, Artificial Sequence
  Synthetic
QQSYSLPPT

SEQ ID NO: 53 - protein, 108 aa, Artificial Sequence
  Synthetic
AIQLTQSPSSLSASIGDRVTITCRASQSVTTYLNWYHQKPGKAPTFLIYGTSALQSGVPSRFSGSGSGTDFTLTISSLQPEDFGIYYCQQSYSLPPTFGGGTNVQIRR

SEQ ID NO: 54 - protein, 5 aa, Artificial Sequence
  Synthetic
SSAVQ

SEQ ID NO: 55 - protein, 17 aa, Artificial Sequence
  Synthetic
WIVVGSGNTNYAQKFQE

SEQ ID NO: 56 - protein, 12 aa, Artificial Sequence
  Synthetic
DGDYDIEGALDY

SEQ ID NO: 57 - protein, 122 aa, Artificial Sequence
  Synthetic
QMQLVQSKPEVKKPGTSVKVSCKASGFTFTSSAVQWVRQARGQRLEWIGWIVVGSGNTNYAQKFQERVTITRDMSTSTAYMELSSLRSEDTAVYYCPRTDGDYDIEGALDYWGQGTLVTVSS

SEQ ID NO: 58 - protein, 11 aa, Artificial Sequence
  Synthetic
RASQSVSSYLA

SEQ ID NO: 59 - protein, 7 aa, Artificial Sequence
  Synthetic
DASNRAT

SEQ ID NO: 60 - protein, 8 aa, Artificial Sequence
  Synthetic
QQRSNWIT

SEQ ID NO: 61 - protein, 107 aa, Artificial Sequence
  Synthetic
EIVLTQSPAILSLSPGERATLSCRASQSVSSYLAWYQQKPGQAPRLLIYDASNRATGIPARFSGSGSGTDFTLTISSLEPEDFAVYYCQQRSNWITFGGGTKVEIKR

SEQ ID NO: 62 - protein, 5 aa, Artificial Sequence
  Synthetic
SYYMH

SEQ ID NO: 63 - protein, 17 aa, Artificial Sequence
  Synthetic
IINPSGGSTSYAQKFQG

SEQ ID NO: 64 - protein, 10 aa, Artificial Sequence
  Synthetic
SGTMTHLKGE

SEQ ID NO: 65 - protein, 119 aa, Artificial Sequence
  Synthetic
EVQLVESGAEVKKPGASVKVSCKASGYTFTSYYMHWVRQAPGQGLEWMGIINPSGGSTSYAQKFQGRVTMTRDTSTSTVYMELSSLRSEDTAVYYCASSGTMTHLKGEWGQGTLVTVSS

SEQ ID NO: 66 - protein, 16 aa, Artificial Sequence
  Synthetic
RSSESLLHSSGYNFLD

SEQ ID NO: 67 - protein, 7 aa, Artificial Sequence
  Synthetic
LGSTRAS

SEQ ID NO: 68 - protein, 8 aa, Artificial Sequence
  Synthetic
MQALETPT

SEQ ID NO: 69 - protein, 112 aa, Artificial Sequence
  Synthetic
DIVMTQSPLSLPVTPGEPASISCRSSESLLHSSGYNFLDWYLQKPGQSPQLLIYLGSTRASGVPDRFSGSGSGTDFTLKISRVDAEDVGVYYCMQALETPTFGQGTKLEIKR

SEQ ID NO: 70 - protein, 9 aa, Artificial Sequence
  Synthetic
SRPVRGAYY

SEQ ID NO: 71 - protein, 118 aa, Artificial Sequence
  Synthetic
QVQLVESGAEVKKPGSSVKVSCKASGGTFSSYAISWVRQAPGQGLEWMGGIIPIFGTANYAQKFQGRVTITADESTSTAYMELSSLRSEDTAVYYCARSRPVRGAYYWGQGTLVTVSS

SEQ ID NO: 72 - protein, 12 aa, Artificial Sequence
  Synthetic
RASQIVNSNYLN

SEQ ID NO: 73 - protein, 7 aa, Artificial Sequence
  Synthetic
GVSNLQV

SEQ ID NO: 74 - protein, 10 aa, Artificial Sequence
  Synthetic
QQSYTTPRYS

SEQ ID NO: 75 - protein, 110 aa, Artificial Sequence
  Synthetic
DIQMTQSPSSLSASVGDRVTITCRASQIVNSNYLNWYQQKPGKAPNLLIYGVSNLQVGVPSRFSGSGSGTDFTLSISSLQVEDSATYYCQQSYTTPRYSFGQGTKLEIRR

SEQ ID NO: 76 - protein, 5 aa, Artificial Sequence
  Synthetic
SYGMH

SEQ ID NO: 77 - protein, 17 aa, Artificial Sequence
  Synthetic
FIRYDGFNKYYADSVKG

SEQ ID NO: 78 - protein, 11 aa, Artificial Sequence
  Synthetic
VGRDGYNWFDY

SEQ ID NO: 79 - protein, 121 aa, Artificial Sequence
  Synthetic
QVQLVESGGGVVQPGGSLRLSCPRTSGFTFSSYGMHWVRQAPGEGLEWVAFIRYDGFNKYYADSVKGRFTISRDNSKNTLYLQLNSLRAEDTAVYYCAKVGRDGYNWFDYWGQGTLVTVSS

SEQ ID NO: 80 - protein, 11 aa, Artificial Sequence
  Synthetic
GGDNIAIKTVH

SEQ ID NO: 81 - protein, 7 aa, Artificial Sequence
  Synthetic
DDSDRPS

SEQ ID NO: 82 - protein, 11 aa, Artificial Sequence
  Synthetic
QVWDSRPDHPV

SEQ ID NO: 83 - protein, 110 aa, Artificial Sequence
  Synthetic
QPVLTQPPSMSVAPGQTARITCGGDNIAIKTVHWYQQRPGQAPVLVVHDDSDRPSGIPERFSGSNSGNTPRTLTISRVEAGDEADYYCQVWDSRPDHPVFGGGTKVTVLR

SEQ ID NO: 84 - protein, 8 aa, Artificial Sequence
  Synthetic
SNRPRTWS

SEQ ID NO: 85 - protein, 18 aa, Artificial Sequence
  Synthetic
RTYYRSKWYNDYAVSVES

SEQ ID NO: 86 - protein, 8 aa, Artificial Sequence
  Synthetic
GSFESTWL

SEQ ID NO: 87 - protein, 121 aa, Artificial Sequence
  Synthetic
QLQLQQSGPGLVKPSQTLSLTCAISGDSVSSNRPRTWSWIRQSPSRGLEWLGRTYYRSKWYNDYAVSVESRIIINPDTSKNQFSLQLNPVTPEDTAVYYCARGSFESTWLWGQGTLVTVSS

SEQ ID NO: 88 - protein, 13 aa, Artificial Sequence
  Synthetic
TRSGGSIANNYVH

SEQ ID NO: 89 - protein, 7 aa, Artificial Sequence
  Synthetic
QDNQRRS

SEQ ID NO: 90 - protein, 9 aa, Artificial Sequence
  Synthetic
QSFDNNNVV

SEQ ID NO: 91 - protein, 110 aa, Artificial Sequence
  Synthetic
QAVVTQPHSVSESPGKTVTISCTRSGGSIANNYVHWYQQRPGSFPTALIYQDNQRRSGVPDRFSGSIDSSSNSASLTISGLKTEDEAEYYCQSFDNNNVVFGGGTQLTVL

SEQ ID NO: 92 - protein, 27 aa, Artificial Sequence
  Synthetic
DLAIVYGSGSYYNHHPRIDYYYYGMDV

SEQ ID NO: 93 - protein, 136 aa, Artificial Sequence
  Synthetic
EVQLVESGAEVKKPGSSVKVSCKASGGTFSSYAISWVRQAPGQGLEWMGGIIPIFGTANYAQKFQGRVAITADESTSTAYMELSSLRSEDTAVYYCARDLAIVYGSGSYYNHHPRIDYYYYGMDVWGQGTTVTVSS

SEQ ID NO: 94 - protein, 14 aa, Artificial Sequence
  Synthetic
AGTSSDIGAYNYVS

SEQ ID NO: 95 - protein, 7 aa, Artificial Sequence
  Synthetic
DVSKRPS

SEQ ID NO: 96 - protein, 10 aa, Artificial Sequence
  Synthetic
FSYAGSYTLV

SEQ ID NO: 97 - protein, 110 aa, Artificial Sequence
  Synthetic
QSVLTQPRSVSGSPGQSVTISCAGTSSDIGAYNYVSWYQQHPGKAPTLMINDVSKRPSGVPDRFSGSKSGNTASLTISGLQAEDEGDYYCFSYAGSYTLVFGGGTQLTVL

SEQ ID NO: 98 - protein, 17 aa, Artificial Sequence
  Synthetic
ADYQWVGAIFRLNAFDI

SEQ ID NO: 99 - protein, 126 aa, Artificial Sequence
  Synthetic
QVQLVESGAEVKKPGASVKVSCKASGYTFTGYYMHWVRQAPGQGLEWMGRINPNSGGTNYAQKFQGRFTISRDNSKTTLFLQMNSLRAEDTAVYYCAKADYQWVGAIFRLNAFDIWGQGTMVTVSS

SEQ ID NO: 100 - protein, 11 aa, Artificial Sequence
  Synthetic
RASQGISNYLA

SEQ ID NO: 101 - protein, 8 aa, Artificial Sequence
  Synthetic
PRTSTLQS

SEQ ID NO: 102 - protein, 9 aa, Artificial Sequence
  Synthetic
QKYNSAPYT

SEQ ID NO: 103 - protein, 108 aa, Artificial Sequence
  Synthetic
VIWMTQSPSSLSASVGDRVTITCRASQGISNYLAWYQQKPGKVPKLLIYPRTSTLQSGVPSRFSGSGSGTDFTLTISSLQPEDVATYYCQKYNSAPYTFGQGTKLEIK

SEQ ID NO: 104 - protein, 381 aa, Artificial Sequence
  Synthetic
METDTLLLWVLLLWVPGSTGAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 105 - protein, 361 aa, Artificial Sequence
  Synthetic
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 106 - protein, 381 aa, Artificial Sequence
  Synthetic
METDTLLLWVLLLWVPGSTGAPASSSTKKTQLQLEHLLLDLQMILNGINNYKNPKLTEMLTFKFYMPKKATELKHLQCLEEELKPLEEVLNLAQSKNFHLTARDAVDNMRVIIQELKGSETTFMCEYADETATIVEFLNRWITFSQSIISTLTDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 107 - protein, 361 aa, Artificial Sequence
  Synthetic
APASSSTKKTQLQLEHLLLDLQMILNGINNYKNPKLTEMLTFKFYMPKKATELKHLQCLEEELKPLEEVLNLAQSKNFHLTARDAVDNMRVIIQELKGSETTFMCEYADETATIVEFLNRWITFSQSIISTLTDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 108 - protein, 509 aa, Artificial Sequence
  Synthetic
METDTLLLWVLLLWVPGSTGEVQLVESGGGLVQPGGSLRLSCPRTSGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKPRTLTISGAQPEDEADYYCALWYSNHWVFGGGTKLTVLEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 109 - protein, 489 aa, Artificial Sequence
  Synthetic
EVQLVESGGGLVQPGGSLRLSCPRTSGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKPRTLTISGAQPEDEADYYCALWYSNHWVFGGGTKLTVLEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 110 - protein, 472 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 111 - protein, 452 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 112 - protein, 625 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 113 - protein, 605 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 114 - protein, 625 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLTARDAVDNMRVIIQELKGSETTFMCEYADETATIVEFLNRWITFSQSIISTLT

SEQ ID NO: 115 - protein, 605 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLTARDAVDNMRVIIQELKGSETTFMCEYADETATIVEFLNRWITFSQSIISTLT

SEQ ID NO: 116 - protein, 471 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 117 - protein, 451 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 118 - protein, 624 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 119 - protein, 604 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 120 - protein, 625 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 121 - protein, 605 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 122 - protein, 625 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTDMLTFKFSMPKKATELKHLQCLEEELKPLEEVLNLAQSKNFHLRPRDLISNINVIVLELKGSETTFMCEYADETATIVEFLNRWITFSQSIISTLT

SEQ ID NO: 123 - protein, 605 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTDMLTFKFSMPKKATELKHLQCLEEELKPLEEVLNLAQSKNFHLRPRDLISNINVIVLELKGSETTFMCEYADETATIVEFLNRWITFSQSIISTLT

SEQ ID NO: 124 - protein, 624 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLTARDAVDNMRVIIQELKGSETTFMCEYADETATIVEFLNRWITFSQSIISTLT

SEQ ID NO: 125 - protein, 604 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLTARDAVDNMRVIIQELKGSETTFMCEYADETATIVEFLNRWITFSQSIISTLT

SEQ ID NO: 126 - protein, 472 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 127 - protein, 452 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 128 - protein, 625 aa, Artificial Sequence; identical to SEQ ID NO: 120
  Synthetic

SEQ ID NO: 129 - protein, 605 aa, Artificial Sequence; identical to SEQ ID NO: 121
  Synthetic

SEQ ID NO: 130 - protein, 625 aa, Artificial Sequence; identical to SEQ ID NO: 122
  Synthetic

SEQ ID NO: 131 - protein, 605 aa, Artificial Sequence; identical to SEQ ID NO: 123
  Synthetic

SEQ ID NO: 132 - protein, 499 aa, Artificial Sequence
  Synthetic
METDTLLLWVLLLWVPGSTGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 133 - protein, 479 aa, Artificial Sequence
  Synthetic
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 134 - protein, 688 aa, Artificial Sequence
  Synthetic
METDTLLLWVLLLWVPGSTGDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSPRTGPRTALALTVDLPPASSEARNSAFGFQGRLLHLSAGQRLGVHLHTEARARHAWQLTQGATVLGLFRVTPEIPAGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 135 - protein, 668 aa, Artificial Sequence
  Synthetic
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSPRTGPRTALALTVDLPPASSEARNSAFGFQGRLLHLSAGQRLGVHLHTEARARHAWQLTQGATVLGLFRVTPEIPAGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 136 - protein, 628 aa, Artificial Sequence
  Synthetic
METDTLLLWVLLLWVPGSTGLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLAGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 137 - protein, 608 aa, Artificial Sequence
  Synthetic
LKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLAGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 138 - protein, 625 aa, Artificial Sequence
  Synthetic
METDTLLLWVLLLWVPGSTGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 139 - protein, 605 aa, Artificial Sequence
  Synthetic
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 140 - protein, 636 aa, Artificial Sequence
  Synthetic
METDTLLLWVLLLWVPGSTGDTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 141 - protein, 616 aa, Artificial Sequence
  Synthetic
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDPRTTYYCQQWSSNPLTFGAGTKLELKEPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 142 - protein, 497 aa, Artificial Sequence
  Synthetic
METDTLLLWVLLLWVPGSTGQVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSSGGGGSGGGSGGGSGGGGSDIQMTQSPSSLSASVGDRVTMTCRASSSVSYMHWYQQTPGKAPKPWIYATSNLASGVPSRFSGSGSGTDYTLTISSLQPEDIATYYCQQWSSNPPTFGQGTKLQITREPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 143 - protein, 477 aa, Artificial Sequence
  Synthetic
QVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSSGGGGSGGGSGGGSGGGGSDIQMTQSPSSLSASVGDRVTMTCRASSSVSYMHWYQQTPGKAPKPWIYATSNLASGVPSRFSGSGSGTDYTLTISSLQPEDIATYYCQQWSSNPPTFGQGTKLQITREPKSSDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 144 - protein, 732 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSSGGGGSGGGSGGGSGGGGSDIQMTQSPSSLSASVGDRVTMTCRASSSVSYMHWYQQTPGKAPKPWIYATSNLASGVPSRFSGSGSGTDYTLTISSLQPEDIATYYCQQWSSNPPTFGQGTKLQITREPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 145 - protein, 712 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSSGGGGSGGGSGGGSGGGGSDIQMTQSPSSLSASVGDRVTMTCRASSSVSYMHWYQQTPGKAPKPWIYATSNLASGVPSRFSGSGSGTDYTLTISSLQPEDIATYYCQQWSSNPPTFGQGTKLQITREPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 146 - protein, 744 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKPRTLTISGAQPEDEADYYCALWYSNHWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 147 - protein, 724 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKPRTLTISGAQPEDEADYYCALWYSNHWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 148 - protein, 745 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFNTYAMNWVRQAPGKGLEWVARIRSKYNNYATYYPRTSVKGRFTISRDDSKNSLFLQMNSLKTEDTAVYYCVRHGNFGNSYVSWFAYWGQGTLVTVSSGGGGSGGGGSGGGGSGGGGSQTVVTQEPSLTVSPGGTVTLTCRSSTGAVTTSNYANWVQEKPDHLFTGLIGGTNKRAPGVPARFSGSLLGGKPRTLTLSGVQPEDEAIYFCALWYSNLWVFGSGTKVTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 149 - protein, 725 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFNTYAMNWVRQAPGKGLEWVARIRSKYNNYATYYPRTSVKGRFTISRDDSKNSLFLQMNSLKTEDTAVYYCVRHGNFGNSYVSWFAYWGQGTLVTVSSGGGGSGGGGSGGGGSGGGGSQTVVTQEPSLTVSPGGTVTLTCRSSTGAVTTSNYANWVQEKPDHLFTGLIGGTNKRAPGVPARFSGSLLGGKPRTLTLSGVQPEDEAIYFCALWYSNLWVFGSGTKVTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 150 - protein, 745 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFNTYAMNWVRQAPGKGLEWVARIRSKYNNYATYYPRTSVKGRFTISRDDSKNSLFLQMNSLKTEDTAVYYCVRHGNFGNSYVSWFAYWGQGTLVTVSSGGGGSGGGGSGGGGSGGGGSQAVVTQEPSLTVSPGGTVTLTCASSTGAVTTSNYANWVQEKPDHLFTGLIGGTNKRAPGVPARFSGSLLGGKPRTLTLSGVQPEDEAEYYCALWYSNLWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 151 - protein, 725 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFNTYAMNWVRQAPGKGLEWVARIRSKYNNYATYYPRTSVKGRFTISRDDSKNSLFLQMNSLKTEDTAVYYCVRHGNFGNSYVSWFAYWGQGTLVTVSSGGGGSGGGGSGGGGSGGGGSQAVVTQEPSLTVSPGGTVTLTCASSTGAVTTSNYANWVQEKPDHLFTGLIGGTNKRAPGVPARFSGSLLGGKPRTLTLSGVQPEDEAEYYCALWYSNLWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 152 - protein, 745 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPRGSLRLSCPRTSGFTFNTYAMNWVRQAPGKGLEWVARIRSKYNNYATYYPRTSVKGRFSISRDDSENALYLQMNSLKTEDTAVYYCVRHGNFGNSYVSWFAYWGQGTLVTVSSGGGGSGGGGSGGGGSGGGGSQTVVTQEPSLTVSPGGTVTLTCRSSTGAVTTSNYANWVQEKPDHLFTGLIGGTNKRAPGVPARFSGSLLGGKPRTLTLSGVQPEDEAIYFCALWYSNLWVFGSGTKVTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 153 - protein, 725 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPRGSLRLSCPRTSGFTFNTYAMNWVRQAPGKGLEWVARIRSKYNNYATYYPRTSVKGRFSISRDDSENALYLQMNSLKTEDTAVYYCVRHGNFGNSYVSWFAYWGQGTLVTVSSGGGGSGGGGSGGGGSGGGGSQTVVTQEPSLTVSPGGTVTLTCRSSTGAVTTSNYANWVQEKPDHLFTGLIGGTNKRAPGVPARFSGSLLGGKPRTLTLSGVQPEDEAIYFCALWYSNLWVFGSGTKVTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 154 - protein, 745 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPRGSLRLSCPRTSGFTFNTYAMNWVRQAPGKGLEWVARIRSKYNNYATYYPRTSVKGRFSISRDDSENALYLQMNSLKTEDTAVYYCVRHGNFGNSYVSWFAYWGQGTLVTVSSGGGGSGGGGSGGGGSGGGGSQAVVTQEPSLTVSPGGTVTLTCASSTGAVTTSNYANWVQEKPDHLFTGLIGGTNKRAPGVPARFSGSLLGGKPRTLTLSGVQPEDEAEYYCALWYSNLWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 155 - protein, 725 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPRGSLRLSCPRTSGFTFNTYAMNWVRQAPGKGLEWVARIRSKYNNYATYYPRTSVKGRFSISRDDSENALYLQMNSLKTEDTAVYYCVRHGNFGNSYVSWFAYWGQGTLVTVSSGGGGSGGGGSGGGGSGGGGSQAVVTQEPSLTVSPGGTVTLTCASSTGAVTTSNYANWVQEKPDHLFTGLIGGTNKRAPGVPARFSGSLLGGKPRTLTLSGVQPEDEAEYYCALWYSNLWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 156 - protein, 468 aa, Artificial Sequence
  Synthetic
METDTLLLWVLLLWVPGSTGQVQLQQWGAGLLKPSETLSLTCAVYGGSFSGYYWSWIRQPPGKGLEWIGEINHSGSTNYNPSLKSRVTISVDTSKNQFSLKLSSVTPRTDTAVYYCARGWYLGFDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 448 aa, Artificial Sequence
  Synthetic
QVQLQQWGAGLLKPSETLSLTCAVYGGSFSGYYWSWIRQPPGKGLEWIGEINHSGSTNYNPSLKSRVTISVDTSKNQFSLKLSSVTPRTDTAVYYCARGWYLGFDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 237 aa, Artificial Sequence
  Synthetic
METDTLLLWVLLLWVPGSTGQSVLTQPRSVSGSPGQSVTISCTGTSRDVGGYDYVSWYQQYPGKAPKLMISGVSERPSGVPDRFTGSRSANTASLTISGLQTDDEANYYCCSYAGSFTWVFGDGTKLTVLGQPKANPTVTLFPPSSEELQANKATLVCLISDFYPGAVTVAWKADGSPVKAGVETTKPSKQSNNKYPRTSSYLSLTPEQWKSHRSYSCQVTHEGSTVEKTVAPTECS

SEQ ID NO: 159 - protein, 217 aa, Artificial Sequence
  Synthetic
QSVLTQPRSVSGSPGQSVTISCTGTSRDVGGYDYVSWYQQYPGKAPKLMISGVSERPSGVPDRFTGSRSANTASLTISGLQTDDEANYYCCSYAGSFTWVFGDGTKLTVLGQPKANPTVTLFPPSSEELQANKATLVCLISDFYPGAVTVAWKADGSPVKAGVETTKPSKQSNNKYPRTSSYLSLTPEQWKSHRSYSCQVTHEGSTVEKTVAPTECS

SEQ ID NO: 160 - protein, 468 aa, Artificial Sequence
  Synthetic
METDTLLLWVLLLWVPGSTGEVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPCSRSTSESTPRTLGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 448 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPCSRSTSESTPRTLGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 162 - protein, 465 aa, Artificial Sequence
  Synthetic
METDTLLLWVLLLWVPGSTGQVQLQQWGAGLLKPSETLSLTCAVYGGSFSGYYWSWIRQPPGKGLEWIGEINHSGSTNYNPSLKSRVTISVDTSKNQFSLKLSSVTPRTDTAVYYCARGWYLGFDYWGQGTLVTVSSASTKGPSVFPLAPCSRSTSESTPRTLGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 445 aa, Artificial Sequence
  Synthetic
QVQLQQWGAGLLKPSETLSLTCAVYGGSFSGYYWSWIRQPPGKGLEWIGEINHSGSTNYNPSLKSRVTISVDTSKNQFSLKLSSVTPRTDTAVYYCARGWYLGFDYWGQGTLVTVSSASTKGPSVFPLAPCSRSTSESTPRTLGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 164 - protein, 725 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFNTYAMNWVRQAPGKGLEWVARIRSKANNYATYYPRTSVKGRFTISRDDSKNSLFLQMNSLKTEDTAVYYCVRHGNFGNSYVSWFAYWGQGTLVTVSSGGGGSGGGGSGGGGSGGGGSQAVVTQEPSLTVSPGGTVTLTCASSTGAVTTSNYANWVQEKPDHLFTGLIGGTNKRAPGVPARFSGSLLGGKPRTLTLSGVQPEDEAEYYCALWYSNLWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 165 - protein, 725 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFNTYAMNWVRQAPGKGLEWVARIRSKANAYATYYPRTSVKGRFTISRDDSKNSLFLQMNSLKTEDTAVYYCVRHGNFGNSYVSWFAYWGQGTLVTVSSGGGGSGGGGSGGGGSGGGGSQAVVTQEPSLTVSPGGTVTLTCASSTGAVTTSNYANWVQEKPDHLFTGLIGGTNKRAPGVPARFSGSLLGGKPRTLTLSGVQPEDEAEYYCALWYSNLWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 166 - protein, 725 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTPRTLGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCPRTSGFTFNTYAMNWVRQAPGKGLEWVARIRSKANAYATYYPRTSVKGRFTISRDDSKNSLFLQMNSLKTEDTAVYYCVRHGNFGNSAVSWFAYWGQGTLVTVSSGGGGSGGGGSGGGGSGGGGSQAVVTQEPSLTVSPGGTVTLTCASSTGAVTTSNYANWVQEKPDHLFTGLIGGTNKRAPGVPARFSGSLLGGKPRTLTLSGVQPEDEAEYYCALWYSNLWVFGGGTKLTVLEPKSCDKTHTCPPCPAPEPRTGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 167 - protein, 431 aa, Artificial Sequence
  Synthetic
QVQLVQSGGTFSSYAISWVRQAPGQGLEWMGGIIPIFGTANYAQKFQGRVTITADESTSTAYMELSSLRSEDTAVYYCARGSGTLNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 217 aa, Artificial Sequence
  Synthetic
QSVLTQPRSVSGSPGQSVTISCTGTSSDIGGYNFVSWYQQHPGKAPKLVITEVSKRPSGVPDRFSGSKSGNTASLTISGLQAEDEADYYCSSNSGDYADVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 169 - protein, 450 aa, Artificial Sequence
  Synthetic
EVQLVQSGAEVKKPGASVKVSCKASGYTFTGYYMHWVRQAPGQGLEWMGRINPNSGGTNYAQKFQGRVTMTRDTSISTAYMELSGLRSDDTAVYFCARVTYNIGWYIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 214 aa, Artificial Sequence
  Synthetic
QAVVTQPPSVSVAPGKTATITCGGNNIGSKGVHWYQQKPGQAPVLVIYYDTDRPSGIPERLSGSNSGNTATLTISRVEAGDEADYYCQVWDSSSDHPVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 171 - protein, 457 aa, Artificial Sequence
  Synthetic
EVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGMWVPSITMIVGGGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 222 aa, Artificial Sequence
  Synthetic
DVVMTQSPLSLPVTPGEPASMSCRSSESLLHRNGFNYLDWYVQRPGQSPKLLIYMGSYRASGVPDRFSGRGSGTDFALKISRVEAEDVGVYYCMQALQSPPLYTFGPGTKLEIKRTVAAPSAVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0368275 A1
(43) Pub. Date: Nov. 7, 2024

(54) ANTI-NECTIN4 ANTIBODIES AND MULTI-SPECIFIC PROTEIN COMPLEXES COMPRISING SUCH

(71) Applicant: Elpis Biopharmaceuticals, Lexington, MA (US)

(72) Inventors: Kehao Zhao, Lexington, MA (US); Yan Chen, Lexington, MA (US); Jenna Nguyen, Lexington, MA (US); Suga Subramaniam, Lexington, MA (US); Ning Jiang, Lexington, MA (US)

(73) Assignee: Elpis Biopharmaceuticals, Lexington, MA (US)

(21) Appl. No.: 18/574,860

(22) PCT Filed: Jun. 28, 2022
(86) PCT No.: PCT/US2022/035363

§ 371 Date: Dec. 28, 2023

Related U.S. Application Data
(60) Provisional application No. 63/216,276, filed on Jun. 29, 2021.

(51) Int. Cl. C07K 16/28; A61K 39/00; A61P 35/00; C07K 16/24; G01N 33/574

(52) U.S. Cl. CPC C07K 16/2803; A61P 35/00; C07K 16/246; C07K 16/2809; C07K 16/2818; C07K 16/2827; C07K 16/2878; G01N 33/57492; A61K 2039/505; C07K 2317/21; C07K 2317/24; C07K 2317/31; C07K 2317/622

(57) ABSTRACT

Antibodies that binds Nectin Cell Adhesion Molecule 4 (nectin-4) and multi-specific protein complexes comprising such anti-nectin4 antibodies, at least one additional antibody moiety binding to another target, and/or at least one cytokine moiety. Also provided herein are pharmaceutical compositions comprising such and uses thereof.

### CROSS REFERENCE TO RELATED APPLICATIONS

This application claims the benefit of the filing date of U.S. Provisional Application No. 63/216,276, filed Jun. 29, 2021, the entire contents of which are incorporated by reference herein.

### BACKGROUND OF THE INVENTION

Nectins and nectin-like molecules are cell adhesion molecules involved in calcium-independent cellular adhesion. Nectin Cell Adhesion Molecule 4 (nectin-4), also known as PVRL4, is a member of the nectin family, which is within the immunoglobulin superfamily. Nectin4 has been reported as a tumor associated antigen in various cancer tissues, including pancreatic cancer, ovarian cancer, lung cancer, and breast cancer. Accordingly, nectin4 may be a promising target in cancer therapy.

### SUMMARY OF THE INVENTION

The present disclosure is based, at least in part, on the development of antibodies binding to nectin4 with high binding affinity and specificity, as well as multi-specific protein complexes comprising such (e.g., bi-specific antibodies and protein complexes comprising an anti-nectin4 moiety and a cytokine (e.g., IL2) moiety).

Accordingly, some aspects of the present disclosure provide an isolated antibody that binds Nectin Cell Adhesion Molecule 4 (nectin-4) (“anti-nectin4 antibody”). The anti-nectin4 antibody binds the same epitope as a reference antibody or competes against the reference antibody from binding to nectin-4. The reference antibody is one of the following: 2020EP034-H09 (a.k.a., EP034-H09), 2020EP034-B09 (a.k.a., EP034-B09), 2020EP034-E01 (a.k.a., EP034-E01), 2020EP47-F02 (a.k.a., EP047-F02), 2021EP030-B10 (a.k.a., EP030-B10), 2021EP030-C11 (a.k.a., EP030-C11), 2021EP030-D06 (a.k.a., EP030-D06), 2021EP030-E10 (a.k.a., EP030-E10), 2021EP030-F02 (a.k.a., EP034-F02), 2021EP030-H06 (a.k.a., EP030-H06), 2021EP029-C04 (a.k.a., EP029-C04), 2021EP032-D10 (a.k.a., EP032-D10), and 2021EP032-E06 (a.k.a., EP032-E06). In specific examples, the reference antibody is EP034-B09.

In some embodiments, the anti-nectin4 antibody may comprise: (a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or (b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody.

In some instances, the anti-nectin4 antibody disclosed herein may comprise HC CDRs that collectively contain no more than 8 amino acid residue variations as compared with the HC CDRs of the reference antibody. Alternatively or in addition, the anti-nectin4 antibody may comprise LC CDRs that collectively contain no more than 8 amino acid residue variations as compared with the LC CDRs of the reference antibody. In some instances, the anti-nectin4 antibody may comprise a V_{H} that is at least 85% identical to the V_{H} of the reference antibody, and/or a V_{L} that is at least 85% identical to the V_{L} of the reference antibody. In some instances, the anti-nectin4 antibody disclosed herein may have a binding affinity of less than about 25 nM to nectin-4 expressed on cell surface. For example, the binding affinity may be less than 10 nM. In some examples, the binding affinity may be less than 1 nM.

In specific examples, the anti-nectin4 antibodies disclosed herein may comprise the same heavy chain complementary determining regions (HC CDRs) and the same light chain complementary determining regions (LC CDRs) as the reference antibodies. In some examples, the anti-nectin4 antibodies comprise the same V_{H} and the same V_{L} as the reference antibodies.

Any of the anti-nectin4 antibodies disclosed herein may be a human antibody or a humanized antibody. In some embodiments, the antibody may be a single-chain antibody (scFv). Alternatively, the antibody may be a multi-chain molecule comprising at least two polypeptides. In some examples, each of the at least two polypeptides comprise an Fc fragment.

In other aspects, the present disclosure features a multi-specific antibody, comprising: (a) a first binding moiety that binds nectin-4; and (b) a second binding moiety that binds CD3. In some instances, the first binding moiety that binds nectin-4 can be any anti-nectin4 antibodies disclosed herein (e.g., derived from clone EP034-B09). In some instances, the second binding moiety that binds CD3 can be derived from clone EP500 or a variant thereof (e.g., EP695, EP696, or EP697). In some embodiments, the first binding moiety, the second binding moiety, or both are in single-chain variable fragment (scFv) format. Alternatively, the first binding moiety, the second binding moiety, or both are in immunoglobulin (Ig) format. In one example, one of the first binding moiety and the second binding moiety is in scFv format and the other binding moiety is in Ig format.

In some examples, (i) the first binding moiety comprises a first heavy chain and a first light chain, wherein the first heavy chain comprises a first heavy chain variable region (V_{H}) and a first heavy chain constant region, which comprises a first Fc fragment; and wherein the first light chain comprises a first light chain variable region (V_{L}) and a first light chain constant region; and (ii) the second binding moiety comprises a second heavy chain and a second light chain, wherein the second heavy chain comprises a second heavy chain variable region (V_{H}) and a second heavy chain constant region, which comprises a second Fc fragment; and wherein the second light chain comprises a second light chain variable region (V_{L}) and a second light chain constant region. The first Fc fragment and the second Fc fragment form a dimer.

In some examples, (i) the first binding moiety comprises a first heavy chain, a second heavy chain, and a light chain, wherein the first heavy chain comprises V_{H} and a first heavy chain constant region, which comprises a first Fc fragment, wherein the second heavy chain comprises the V_{H} and a second heavy chain constant region, which comprises a second Fc fragment, and wherein the light chain comprises a V_{L} and a light chain constant region; and (ii) the second binding moiety is an scFv fragment, which is fused with either the first heavy chain or the second heavy chain of (i), optionally wherein the scFv fragment is fused with the first or second heavy chain between the first or second Fc fragment and the V_{H}. The first Fc fragment and the second Fc fragment form a dimer.

In some examples, (i) the first binding moiety comprises a first heavy chain, a second heavy chain, and a light chain, wherein the first heavy chain comprises V_{H} and a first heavy chain constant region, which comprises a first Fc fragment, and wherein the second heavy chain comprises the V_{H} and a second heavy chain constant region, which comprises a second Fc fragment, and wherein the light chain comprises a V_{L} and a light chain constant region; and (ii) the second binding moiety is a heavy chain only fragment (VHH), which is fused with either the first heavy chain or the second heavy chain of (i), optionally wherein the VHH fragment is fused with the first or second heavy chain between the first or second Fc fragment and the V_{H}. The first Fc fragment and the second Fc fragment form a dimer.

In some examples, (i) the first binding moiety comprises a first heavy chain and a first light chain, wherein the first heavy chain comprises a first heavy chain variable region (V_{H}) and a first heavy chain constant region, which comprises a first Fc fragment; and wherein the first light chain comprises a first light chain variable region (V_{L}) and a first light chain constant region; and (ii) the second binding moiety is an scFv fragment fused to a second Fc fragment. The first Fc fragment and the second Fc fragment form a dimer.

Any of the multi-specific antibodies disclosed herein may further comprise a cytokine, which optionally is IL-2. In some embodiments, the cytokine is fused to the C-terminus of the first Fc fragment. In some embodiments, the cytokine is fused to the C-terminus of the second Fc fragment. In some embodiments, the cytokine is fused to both the C-terminus of the first Fc fragment and the C-terminus of the second Fc fragment.

In some embodiments, the multi-specific antibody disclosed herein may further comprise a third binding moiety, which binds a T cell co-stimulatory receptor. Examples include, but are not limited to, ICOS, 4-1BB, CD28, or CD86.

In another aspect, the present disclosure features a protein complex, comprising a first moiety that binds nectin-4 and a second moiety that comprises a cytokine, e.g., IL-2. The first moiety that binds nectin-4 may be any of the anti-nectin4 antibodies disclosed herein. In some embodiments, the first moiety comprises an scFv fragment fused to a first Fc fragment; and the second moiety comprises the cytokine fused to a second Fc fragment. The first Fc fragment and the second Fc fragment form a dimer.

In some embodiments, the first moiety comprises a first polypeptide, which comprises an scFv fragment fused to a first Fc fragment, and a second polypeptide, which comprises the scFv fragment fused to a second Fc fragment. In some examples, the cytokine of the second moiety is fused to the C-terminus of the first Fc fragment. In some examples, the cytokine of the second moiety is fused to the C-terminus of the second Fc fragment. In some examples, the cytokine of the second moiety is fused to both the C-terminus of the first Fc fragment and the C-terminus of the second Fc fragment. The first Fc fragment and the second Fc fragment form a dimer.

In some examples, (i) the first moiety comprises a heavy chain comprising a V_{H} and a heavy chain constant region, which comprises a first Fc fragment, and a light chain comprising a V_{L} and a light chain constant region; and (ii) the second moiety comprises the cytokine fused to a second Fc fragment. The first Fc fragment and the second Fc fragment form a dimer.

In some examples, the first moiety comprises a first heavy chain comprising a V_{H} and a first heavy chain constant region, which comprises a first Fc fragment, a second heavy chain comprising the V_{H} and a second heavy chain constant region, which comprises a second Fc fragment, and a light chain comprising a V_{L} and a light chain constant region. In some examples, the cytokine of the second moiety is fused to the C-terminus of the first Fc fragment. In some examples, the cytokine of the second moiety is fused to the C-terminus of the second Fc fragment. In some examples, the cytokine of the second moiety is fused to both the C-terminus of the first Fc fragment and the C-terminus of the second Fc fragment. The first Fc fragment and the second Fc fragment form a dimer.

In any of the multi-specific antibodies and protein complexes disclosed herein, the first Fc fragment and the second Fc fragment comprise mutations that enhances heterodimeration over homodimeration as relative to the wild-type counterpart. In some embodiments, the mutations are knob-hole mutations. For example, the knob mutation may comprise S354C, T366W and/or K409A. The hole mutation may comprise S354C, Y349C, T366S, L368A, F405K, and/or Y407V.

In addition, the present disclosure provides s nucleic acid or a set of nucleic acids, which collectively encodes any of the nectin4 antibodies disclosed here or any of the multi-specific antibodies or protein complexes as also disclosed herein. In some embodiments, the nucleic acid or the set of nucleic acids can be a vector or a set of vectors. In some examples, the vector is an expression vector. Further, provided herein is a host cell comprising any of the encoding nucleic acid or the set of nucleic acids as disclosed herein.

Moreover, provided herein is a pharmaceutical composition comprising any of the anti-nectin4 or multi-specific antibodies disclosed herein, any of the protein complexes disclosed herein, any of the encoding nucleic acid or nucleic acids, or the host cell comprising such, and a pharmaceutically acceptable carrier.

In other aspects, the present disclosure features a method for inhibiting nectin-4 or nectin-4⁺ cells in a subject, comprising administering to a subject in need thereof any effective amount of the pharmaceutical composition disclosed herein. In some embodiments, the subject is a human patient having nectin-4⁺ pathogenic cells. In some examples, the subject is a human patient having nectin-4 positive cancer. Examples include breast cancer, bladder cancer, ovary cancer, cervical cancer, pancreatic cancer, lung cancer, or head and neck cancer.

Further, the present disclosure features a method for detecting presence of nectin, comprising: (i) contacting an antibody that binds nectin4 as disclosed herein with a sample suspected of containing nectin-4, and (ii) detecting binding of the antibody to nectin-4. In some instances, the antibody is conjugated to a detectable label. In some instances, the nectin-4 is expressed on cell surface. In some instances, the contacting step is performed by administering the antibody to a subject.

In addition, the present disclosure provides a method of producing an antibody binding to nectin-4 or a multi-specific antibody or protein complex comprising such, comprising: (i) culturing the host cell comprising nucleic acid(s) encoding the anti-nectin4 antibody, the multi-specific antibody, or the protein complex as disclosed herein under conditions allowing for expression of the antibody that binds nectin-4, the multi-specific antibody comprising such, or the protein complex comprising such; and (ii) harvesting the antibody, the multi-specific antibody, or the protein complex thus produced from the cell culture.

Also within the scope of the present disclosure are any of the anti-nectin4 antibodies, the multi-specific antibodies, and the protein complexes disclosed herein for use in treating a target disease (e.g., a disease or disorder associated with nectin4⁺ cells such as nectin4⁺ cancer cells) and use of such antibody, multi-specific antibody, or protein complex for manufacturing a medicament for use in treating the target disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram showing antibody-dependent cell cytotoxicity (ADCC) of anti-nectin4 IgG antibodies against cells expressing nectin4.

FIGS. 2A-2E include diagrams illustrating exemplary bispecific antibodies comprising an anti-nectin4 arm, an anti-CD3 arm, and optionally a cytokine moiety. FIG. 2A: anti-nectin4/CD3 bispecific antibody. FIGS. 2A-2C: anti-nectin4/CD3 bispecific antibodies further comprising two cytokines or two copies of a cytokine. FIGS. 2D-2E: anti-nectin4/CD3 bispecific antibodies further comprising a cytokine.

FIGS. 3A-3F include diagrams illustrating exemplary anti-nectin4/cytokine protein complexes. FIGS. 3A, 3C, 3D, and 3F: anti-nectin4 antibody complexed with a cytokine.

FIGS. 3B and 3E: anti-nectin4 antibody complexed with two cytokines or two copies of a cytokine.

FIG. 4 is a diagram showing internalization of various anti-nectin4 antibodies as indicated to CHOK cells.

FIGS. 5A-5E include diagrams showing cytotoxicity of bispecific antibody EP457/EP378/EP289 against cancer cells. FIG. 5A: MCF7 cells. FIG. 5B: T47D cells.

FIG. 5C: T47D cells in PBMC. FIG. 5D: T47D cells in PBMC at 28-hour co-culturing.

FIG. 5E: T47D cells in PBMC at 60 hours coculturing.

FIGS. 6A and 6B include diagrams showing cytokine release. FIG. 6A: IFNγ.

FIG. 6B: TNFα.

FIGS. 7A-7D include diagrams showing p-STAT5 activation by anti-nectin4/IL2 protein complexes. FIG. 7A: CD4⁺/FOXP3⁻ T cells. FIG. 7B: CD8⁺ T cells. FIG. 7C: NK Cells. FIG. 7D: T_{reg} Cells.

FIGS. 8A and 8B include diagrams showing cytotoxic T lymphocyte activity of anti-nectin4/CD3/IL2 protein complexes. FIG. 8A: cell lysis levels. FIG. 8B: IFNγ secretion levels.

FIGS. 9A and 9B include diagrams showing in vivo anti-tumor activity of anti-nectin4/CD3 bispecific antibodies. FIG. 9A: tumor volume. FIG. 9B: animal body weight.

### DETAILED DESCRIPTION OF THE INVENTION

Provided herein are antibodies capable of binding to human nectin4 polypeptide (“anti-nectin4 antibodies), including nectin4 expressed on cell surface, and multi-specific antibodies and protein complexes comprising such an anti-nectin4 antibody. The anti-nectin4 antibodies disclosed herein show high binding affinity and specificity to human nectin4. Such antibodies, in IgG form, showed high cytotoxicity against nectin4-positive cells in vitro. Multi-specific antibodies and protein complexes comprising the anti-nectin4 antibodies, an anti-CD3 moiety, and/or a cytokine moiety (IL-2) showed both high in vitro cytotoxic T lymphocyte (CTL) activity and capability of activating immune cells (e.g., T cells and NK cells), indicating dual functionality in therapeutic uses.

Nectin4 is one of the five members of the Nectin family, which belongs to the immunoglobulin superfamily. Nectin4 includes three conserved immunoglobulin-like domains in its extracellular region. Nectin4 from various species are well known in the art. For example, the amino acid sequence of human nectin4 can be found under GenBank accession no. NM_030916 (see also Gene ID: 81607).

Several reports show that expression of nectin4 is associated with a number of cancer tissues, including pancreatic, ovarian, lung and breast cancers. Zeindler et al., Front. Med. 6:200. doi: 10.3389/fmed.2019.00200. Nectin4 was also reported to be a target for melanoma. Tanaka et al., 2021; 22(2):976. Accordingly, the anti-nectin4 antibodies and multi-specific protein complexes comprising such as disclosed herein can be used for treating diseases associated with nectin4. In addition, the anti-nectin4 antibodies can also be used as diagnostic agents for detecting presence of nectin4, including nectin4⁺ cells. The molecules disclosed herein may also be used for research purposes.

### I. Antibodies Binding to Nectin4

The present disclosure provides antibodies binding to nectin4, for example, human nectin4. In some embodiments, the anti-nectin4 antibodies disclosed herein are capable of binding to nectin4 expressed on cell surface (e.g., binding to nectin4⁺ cells). As such, the antibodies disclosed herein may be used for either therapeutic or diagnostic purposes to target nectin4-positive cells (e.g., cancer cells). As used herein, the term “anti-nectin4 antibody” refers to any antibody capable of binding to a nectin4 polypeptide (e.g., a nectin4 polypeptide expressed on cell surface), which can be of a suitable source, for example, human or a non-human mammal (e.g., mouse, rat, rabbit, primate such as monkey, etc.).

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-nectin4 antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies (e.g., antibody-drug conjugates or ADCs). An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

The anti-nectin4 antibody described herein may be a full-length antibody, which contains two heavy chains and two light chains, each including a variable domain and a constant domain. Alternatively, the anti-nectin4 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., anti-nectin4 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-nectin4 antibodies are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-nectin4 antibodies may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-nectin4 antibody disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-nectin4 antibodies described herein specifically bind to the corresponding target antigen (e.g., nectin4) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (nectin4) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some embodiments, an anti-nectin4 antibody as described herein has a suitable binding affinity for the target antigen (e.g., nectin4) or antigenic epitopes thereof. As used herein, “binding affinity” refers to the apparent association constant or K_{A}. The K_{A} is the reciprocal of the dissociation constant (K_{D}). The anti-nectin4 antibody described herein may have a binding affinity (K_{D}) of at least 100 nM, 10 nM, 1 nM, 0.1 nM, or lower for nectin4. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the anti-nectin4 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-nectin4 antibody disclosed herein has an EC₅₀ value of lower than 10 nM, e.g., <1 nM, <0.5 nM, or lower than 0.1 nM, for binding to nectin4 and/or nectin4-positive cells. As used herein, EC₅₀ values refer to the minimum concentration of an antibody required to bind to 50% of the cells in a nectin4-positive cell population. EC₅₀ values can be determined using conventional assays and/or assays disclosed herein. See, e.g., Examples below.

A number of exemplary anti-nectin4 antibodies are provided in Sequence Table 1 below (CDRs indicated in boldface as determined by the Kabat scheme. See, e.g., Kabat, E. A., et al. (1991) Sequences of Proteins of Immunological Interest, Fifth Edition, U.S. Department of Health and Human Services, NIH Publication No. 91-3242. See also www2.mrc-lmb.cam.ac.uk/vbase/alignments2.php.

In some embodiments, the anti-nectin4 antibodies described herein bind to the same epitope of a nectin4 polypeptide as any of the exemplary antibodies described herein (for example, 2020EP034-B09, 2021EP023-D06, 2021EP030-F02, 2020EP034-E01) or compete against the exemplary antibody from binding to the nectin4 antigen. An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

In some examples, the anti-nectin4 antibody comprises the same V_{H} and/or V_{L} CDRs as an exemplary antibody described herein. See Sequence Table 1. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-nectin4 antibodies may have the same V_{H}, the same V_{L}, or both as compared to an exemplary antibody described herein.

Also within the scope of the present disclosure are functional variants of any of the exemplary anti-nectin4 antibodies as disclosed herein. Such functional variants are substantially similar to the exemplary antibody, both structurally and functionally. A functional variant comprises substantially the same V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same epitope of nectin4 with substantially similar affinity (e.g., having a K_{D} value in the same order). In some instances, the functional variants may have the same heavy chain CDR3 as the exemplary antibody, and optionally the same light chain CDR3 as the exemplary antibody. Alternatively or in addition, the functional variants may have the same heavy chain CDR2 as the exemplary antibody. Such an anti-nectin4 antibody may comprise a V_{H} fragment having CDR amino acid residue variations in only the heavy chain CDR1 as compared with the V_{H} of the exemplary antibody. In some examples, the anti-nectin4 antibody may further comprise a V_{L} fragment having the same V_{L} CDR3, and optionally same V_{L} CDR1 or V_{L} CDR₂ as the exemplary antibody.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-nectin4 antibody may comprise heavy chain CDRs that are at least 80% (e.g., 85%, 90%, 95%, or 98%) sequence identity, individually or collectively, as compared with the V_{H} CDRs of an exemplary antibody described herein. Alternatively or in addition, the anti-nectin4 antibody may comprise light chain CDRs that are at least 80% (e.g., 85%, 90%, 95%, or 98%) sequence identity, individually or collectively, as compared with the V_{L} CDRs as an exemplary antibody described herein. As used herein, “individually” means that one CDR of an antibody shares the indicated sequence identity relative to the corresponding CDR of the exemplary antibody. “Collectively” means that three V_{H} or V_{L} CDRs of an antibody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-nectin4 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-nectin4 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some embodiments, the anti-nectin antibody disclosed herein may be a single chain antibody (scFv). A scFv antibody may comprise a V_{H} fragment and a V_{L} fragment, which may be linked via a flexible peptide linker. In some instances, the scFv antibody may be in the V_{H}→V_{L} orientation (from N-terminus to C-terminus). In other instances, the scFv antibody may be in the V_{L}→V_{H} orientation (from N-terminus to C-terminus). Exemplary anti-nectin4 scFv antibodies include those having the V_{H}/V_{L} pair of any of the exemplary anti-nectin4 antibodies listed in Sequence Table 1.

Any of the anti-nectin4 antibody as described herein, e.g., the exemplary anti-nectin4 antibodies provided here, can bind and inhibit (e.g., reduce or eliminate) the activity of nectin4-positive cells (e.g., B cells). In some embodiments, the anti-nectin4 antibody as described herein can bind and inhibit the activity of nectin4-positive cells by at least 30% (e.g., 35%, 40%, 45%, 50%, 60%, 70%, 80%, 90%, 95% or greater, including any increment therein). The inhibitory activity of an anti-nectin4 antibody described herein can be determined by routine methods known in the art, e.g., by an assay for measuring the K_{i,}^{app} value.

In some examples, the K_{i,}^{aPP} value of an antibody may be determined by measuring the inhibitory effect of different concentrations of the antibody on the extent of a relevant reaction; fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{( [ E ] - [ I ] - {K}_{i}^{app} ) + \sqrt{{( [ E ] - [ I ] - {K}_{i}^{app} )}^{2} + 4 [ E ] \cdot } {K}_{i}^{app}}{2} \qquad ( Equation 1 )$$

Where A is equivalent to νₒ/E, the initial velocity (νₒ) of the enzymatic reaction in the absence of inhibitor (1) divided by the total enzyme concentration (E). In some embodiments, the anti-nectin4 antibody described herein may have a Ki^{app} value of 1000, 500, 100, 50, 40, 30, 20, 10, 5 pM or less for the target antigen or antigen epitope.

### II. Multi-Specific Protein Complexes

Any of the anti-nectin4 antibodies disclosed herein may be used to construct multi-specific antibodies or protein complexes comprising such. As used herein, “multi-specific antibodies” refers to a protein molecule comprising at least two antibody moieties binding to at least two different antigens or antigen epitopes. The multi-specific antibodies disclosed herein may further comprise a non-antibody moiety such as a cytokine moiety as disclosed herein. The “protein complex” (also named as multi-specific protein complex) refers to a protein molecule comprising an anti-nectin4 antibody as disclosed herein and a non-antibody moiety such as a cytokine moiety as disclosed herein.

### (A) Multi-Specific Antibodies

In some embodiments, the multi-specific antibody disclosed herein may be a bi-specific antibody comprising a first binding moiety that binds nectin4 and a second binding moiety that binds CD3. Any of the anti-nectin4 antibodies disclosed herein may be used for constructing such a bi-specific antibody. See above descriptions and Sequence Tables 1 and 2 below. Any anti-CD3 antibodies known in the art may be used as the second binding moiety, for example, the exemplary anti-CD3 antibodies provided in Sequence Table 2 below (e.g., the OKT3 antibody and SP34 antibody. See, e.g., polypeptides EP369 and EP437). In some examples, the anti-CD3 antibody may be a humanized version of the OKT3 or SP34. For example, the anti-CD3 antibody can be EP500, which is a humanized version of SP34, or a variant thereof (e.g., EP695, EP696, or EP697).

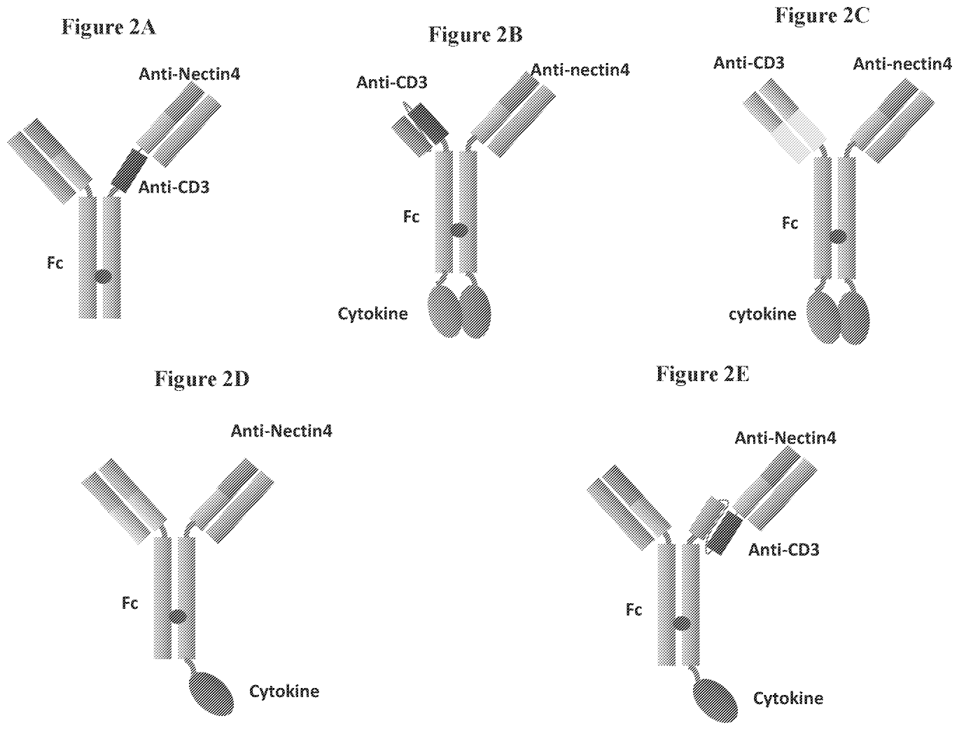

The anti-nectin4/CD3 bispecific antibodies disclosed herein may be in any format known in the art or disclosed in Examples below. See, e.g., FIGS. 2A to 2E. In some embodiments, one or both of the anti-nectin4 and anti-CD3 moieties can be in a single-chain variable fragment (scFv) format. Alternatively, one or more both of the anti-nectin4 and anti-CD3 moieties are in an immunoglobulin (Ig) format (e.g., comprising a heavy chain variable region or light chain variable region linked to the corresponding constant region or a fragment thereof). In other embodiments, one or more both of the anti-nectin4 and anti-CD3 moieties are in a Fab format. In some instances, one of the anti-nectin4 and anti-CD3 moieties can be in one format (e.g., scFv, Ig, VHH, or Fab) and the other binding moiety can be in a different format (e.g., scFv, Ig, VHH, or Fab). In one specific example, one of the anti-nectin4 and anti-CD3 moieties (e.g., the anti-nectin4 moiety) can be in scfv format and the other binding moiety (e.g., the anti-CD3 moiety) can be in Ig format.

In some examples, the anti-nectin4 binding moiety may comprise a first heavy chain and a first light chain. The first heavy chain comprises a first heavy chain variable region (V_{H}) and a first heavy chain constant region, which comprises a first Fc fragment. The first light chain comprises a first light chain variable region (V_{L}) and a first light chain constant region. The second binding moiety (e.g., anti-CD3) comprises a second heavy chain and a second light chain. The second heavy chain comprises a second heavy chain variable region (V_{H}) and a second heavy chain constant region, which comprises a second Fc fragment. The second light chain comprises a second light chain variable region (V_{L}) and a second light chain constant region. The first Fc fragment and the second Fc fragment form a dimer. One example is provided in FIGS. 2C and 2D.

In some examples, the anti-nectin4 binding moiety may comprise a first heavy chain, a second heavy chain, and a light chain. The first heavy chain comprises a V_{H} and a first heavy chain constant region, which comprises a first Fc fragment. The second heavy chain comprises the V_{H} and a second heavy chain constant region, which comprises a second Fc fragment. The light chain comprises a V_{L} and a light chain constant region. The second binding moiety (e.g., binding to CD3) is an scFv fragment, which is fused with either the first heavy chain or the second heavy chain of the anti-nectin4 binding moiety. In some examples, the scFv fragment is fused with the first or second heavy chain between the first or second Fc fragment and the V_{H}. The first Fc fragment and the second Fc fragment form a dimer. One example is provided in FIG. 2E.

In some examples, the anti-nectin4 binding moiety may comprise a first heavy chain, a second heavy chain, and a light chain. The first heavy chain comprises V_{H} and a first heavy chain constant region, which comprises a first Fc fragment. The second heavy chain comprises the V_{H} and a second heavy chain constant region, which comprises a second Fc fragment. The light chain comprises a V_{L} and a light chain constant region. The second binding moiety (e.g., anti-CD3 binding moiety) is a heavy chain only fragment (VHH), which is fused with either the first heavy chain or the second heavy chain of the anti-nectin4 binding moiety. The VHH fragment is fused with the first or second heavy chain between the first or second Fc fragment and the V_{H}. The first Fc fragment and the second Fc fragment form a dimer. One example is provided in FIG. 2A.

In some examples, the anti-nectin4 binding moiety may comprise a first heavy chain and a first light chain. The first heavy chain comprises a first heavy chain variable region (V_{H}) and a first heavy chain constant region, which comprises a first Fc fragment. The first light chain comprises a first light chain variable region (V_{L}) and a first light chain constant region. The second binding moiety is an scFv fragment fused to a second Fc fragment. The first Fc fragment and the second Fc fragment form a dimer. One example is provided in FIG. 2B.

In some instances, the Fc fragments in any of the bi-specific antibodies disclosed herein may comprise one or more mutations to enhance heterodimer formation (between the two polypeptides of the bispecific antibody) and reduce or eliminate formation of homodimers (between two copies of one polypeptide of the bispecific antibody). In some examples, the Fc fragments in any of the bispecific antibodies disclosed herein may comprise one or more knob/hole modifications in the CH2 domain, in the CH3 domain, or in both the CH2 and CH3 domains. Typically, the terms “a knob and a hole” or “knobs-into-holes” are used interchangeably herein. Knobs-into-holes amino acid changes is a rational design strategy known in the art for heterodimerization of the heavy (H) chains in the production of bispecific IgG antibodies. Carter, J. Immunol. Methods, 248(1-2):7-15 (2001), the relevant disclosures of which are incorporated by reference herein for the purpose and subject matter referenced herein. Exemplary knob-hole mutations include S354C, T366W, K409A, Y349C, T366S, L368A, F405K, Y407V or a combination thereof. Positions of the noted amino acid residues follow the EU numbering system.

In some examples, a bispecific antibody for binding to nectin4 and CD3 as disclosed herein may comprise a binding moiety to nectin4 that is described from EP034-B09 and a binding moiety to CD3 that is derived from the OKT3 antibody (e.g., humanized version or functional variants such as EP369, EP456, or EP457). In other examples, a bispecific antibody for binding to nectin4 and CD3 as disclosed herein may comprise a binding moiety to nectin4 that is described from EP034-B09 and a binding moiety to CD3 that is derived from the SP34 antibody (humanized version or functional variants such as EP499, EP500, EP695, EP696, or EP697). Exemplary anti-nectin4/CD3 bispecific antibodies may comprise the polypeptides of (a) SEQ ID NOs: 109, 111, 170, (b) SEQ ID NOs: 143, 111, and 170; (c) SEQ ID NOs: 145, 127, and 170; (d) SEQ ID NOs: 147, 127, and 170; (e) SEQ ID NOs: 149, 127, and 170; (f) SEQ ID NOs: 151, 127, and 170; or (g) SEQ ID NOs: 109, 113, and 170. Other examples can be found in Examples below, all of which are within the scope of the present disclosure.

In some embodiments, the anti-nectin4/CD3 antibodies may further comprise one or more additional binding moieties, which may bind to one or more immune cell receptors, for example, ICOS, 4-1BB, CD28, and/or CD86.

In some embodiments, one or more chains of the multi-specific antibodies disclosed herein may further comprise a cytokine moiety, such as IL-2.

### (B) Protein Complex

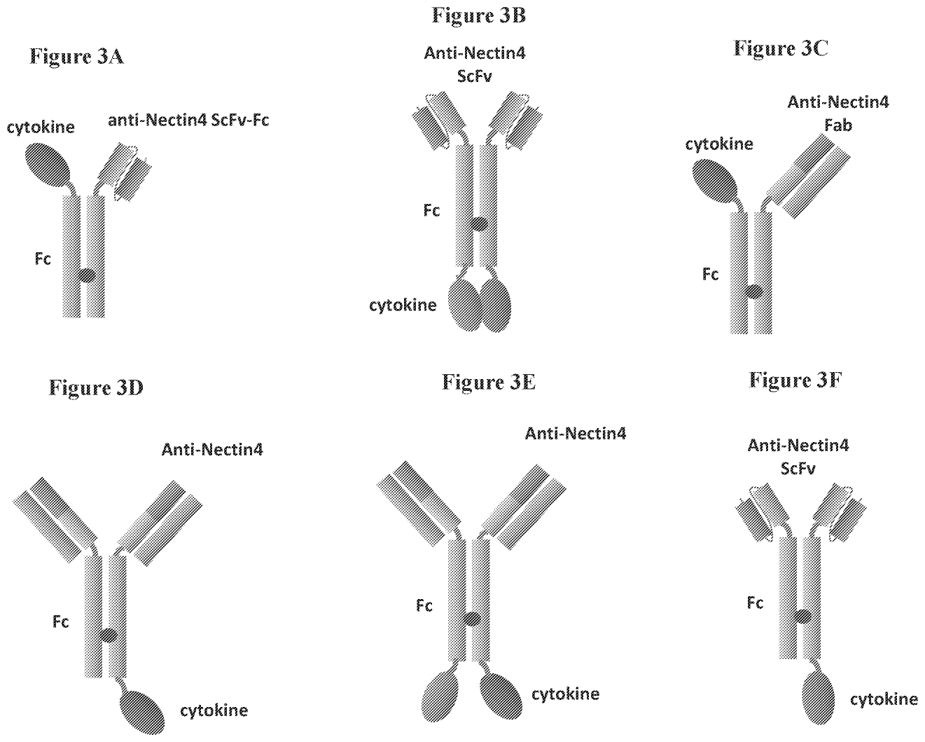

Any of the anti-nectin4 antibodies can also be used to make protein complexes comprising the anti-nectin4 binding moiety and one or more cytokines, which may be IL-2. Some examples are provided in FIGS. 3A-3F. The anti-nectin4 binding moiety may be in any suitable antibody format and the cytokine moieties may be fused to one or more chains of the antibody moiety.

In some examples, the anti-nectin4 binding moiety can comprise an scFv fragment fused to a first Fc fragment; and the cytokine can be fused to a second Fc fragment. The first Fc fragment and the second Fc fragment form a dimer. See, e.g., FIG. 3A.

In some examples, the anti-nectin4 binding moiety may comprise a first polypeptide, which comprises an scFv fragment fused to a first Fc fragment, and a second polypeptide, which comprises the scFv fragment fused to a second Fc fragment. The cytokine can be fused to the C-terminus of the first Fc fragment. Alternatively, the cytokine can be fused to the C-terminus of the second Fc fragment. In some instances, the cytokine can be fused to both the first and second Fc fragments. The first Fc fragment and the second Fc fragment form a dimer. See, e.g., FIGS. 3B and 3F.

In some examples, the anti-nectin4 binding moiety may comprise a heavy chain comprising a V_{H} and a heavy chain constant region, which comprises a first Fc fragment, and a light chain comprising a V_{L} and a light chain constant region. The cytokine can be fused to a second Fc fragment. The first Fc fragment and the second Fc fragment form a dimer. See, e.g., FIG. 3C.

In some examples, the anti-nectin4 binding moiety may comprise a first heavy chain comprising a V_{H} and a first heavy chain constant region, which comprises a first Fc fragment, a second heavy chain comprising the V_{H} and a second heavy chain constant region, which comprises a second Fc fragment, and a light chain comprising a V_{L} and a light chain constant region. The cytokine may be fused to the C-terminus of the first Fc fragment, the C-terminus of the second Fc fragment, or both. The first Fc fragment and the second Fc fragment form a dimer. See, e.g., FIGS. 3D and 3E.

In some instances, the Fc fragments in any of the bi-specific antibodies disclosed herein may comprise one or more mutations to enhance heterodimer formation (between the two polypeptides of the bispecific antibody) and reduce or eliminate formation of homodimers (between two copies of one polypeptide of the bispecific antibody). In some examples, the Fc fragments in any of the bispecific antibodies disclosed herein may comprise one or more knob/hole modifications in the CH2 domain, in the CH3 domain, or in both the CH2 and CH3 domains. Exemplary knob/hole modifications are provided herein, any of which can be used in the protein complexes disclosed herein.

Exemplary protein complexes as disclosed herein may comprise the polypeptides of (a) SEQ ID NOs: 109, 129, and 170; (b) SEQ ID NOs: 127, 129, and 170; (c) SEQ ID NOs: 147, 127, and 170; (d) SEQ ID NOs: 164, 127, and 170; (e) SEQ ID NOs: 165, 127, and 170; or (f) SEQ ID NOs: 166, 127, and 170. Other examples are provided in Examples below, all of which are within the scope of the present disclosure.

### III. Preparation of Anti-Nectin4 Antibodies and Protein Complexes Comprising Such

Antibodies capable of binding nectin4 as described herein can be made by any method known in the art. See, for example, Harlow and Lane, (1998) Antibodies: A Laboratory Manual, Cold Spring Harbor Laboratory, New York. In some embodiments, the antibody may be produced by the conventional hybridoma technology. Alternatively, the anti-nectin4 antibody may be identified from a suitable library (e.g., a human antibody library).

In some instances, high affinity fully human nectin4 binders may be obtained from a human antibody library following conventional screening strategies. See also Example 1 below. This strategy allows for maximizing the library diversity to cover board and active epitopes on nectin4 expressing cells.

If desired, an antibody (monoclonal or polyclonal) of interest (e.g., produced by a hybridoma cell line or isolated from an antibody library) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to, e.g., humanize the antibody or to improve the affinity (affinity maturation), or other characteristics of the antibody. For example, the constant region may be engineered to more resemble human constant regions to avoid immune response if the antibody is from a non-human source and is to be used in clinical trials and treatments in humans. Alternatively or in addition, it may be desirable to genetically manipulate the antibody sequence to obtain greater affinity and/or specificity to the target antigen and greater efficacy in enhancing the activity of nectin4. It will be apparent to one of skill in the art that one or more polynucleotide changes can be made to the antibody and still maintain its binding specificity to the target antigen.

Alternatively, antibodies capable of binding to the target antigens as described herein (a nectin4 molecule) may be isolated from a suitable antibody library via routine practice. Antibody libraries can be used to identify proteins that bind to a target antigen (e.g., human nectin4 such as cell surface nectin4) via routine screening processes. In the selection process, the polypeptide component is probed with the target antigen or a fragment thereof and, if the polypeptide component binds to the target, the antibody library member is identified, typically by retention on a support. Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

There are a number of routine methods known in the art to identify and isolate antibodies capable of binding to the target antigens described herein, including phage display, yeast display, ribosomal display, or mammalian display technology. In some embodiments, mRNA display maybe used for isolating anti-nectin4 antibodies. See Example 1 below.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries).

Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of nectin4 have been replaced (swapped) with sequences from a closely related, but antigenically distinct protein (such as another member of the tumor necrosis factor receptor family). By assessing binding of the antibody to the mutant nectin4, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-nectin4 antibody or a multi-specific protein complex comprising such as disclosed herein can be prepared by recombinant technology as exemplified below.

Nucleic acids encoding the heavy and light chain of an anti-nectin4 antibody as described herein or nucleic acids encoding the multiple polypeptides of a multi-specific protein complex as also disclosed herein can be cloned into one expression vector, each nucleotide sequence being in operable linkage to a suitable promoter. In one example, each of the nucleotide sequences encoding the heavy chain and light chain or the multiple polypeptides is in operable linkage to a distinct prompter. Alternatively, the encoding nucleotide sequences can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two or more chains of the antibody or the multi-specific protein complex are cloned into two or more vectors, which can be introduced into the same or different cells. When the two or more chains are expressed in different cells, each of them can be isolated from the host cells expressing such and the isolated multiple chains can be mixed and incubated under suitable conditions allowing for the formation of the antibody or the multi-specific protein complex.

Generally, a nucleic acid sequence encoding one or all chains of an antibody or a multi-specific protein complex can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies or the multi-specific protein complexes may be introduced into suitable host cells for producing the antibodies. The host cells can be cultured under suitable conditions for expression of the antibody, the multi-specific complex, or any polypeptide chain thereof. Such antibodies, protein complexes, or polypeptide chains thereof can be recovered by the cultured cells (e.g., from the cells or the culture supernatant) via a conventional method, e.g., affinity purification. If necessary, polypeptide chains of the antibody or protein complex can be incubated under suitable conditions for a suitable period of time allowing for production of the antibody.

In some embodiments, methods for preparing an antibody or multi-specific protein complex described herein involve a recombinant expression vector that encodes both the heavy chain and the light chain of an anti-nectin antibody, as also described herein, and optionally chains of a second antibody and/or chain(s) of a cytokine. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two or more polypeptide chains that form the antibody or the multi-specific protein complex, which can be recovered from the cells or from the culture medium. When necessary, the two or more chains recovered from the host cells can be incubated under suitable conditions allowing for the formation of the antibody or the multi-specific protein complex.

In one example, two recombinant expression vectors are provided, one encoding the heavy chain of the anti-nectin4 antibody and the other encoding the light chain of the anti-nectin4 antibody. Alternatively, two or more recombinant expression vectors are provided, each encoding one chain of a multi-specific protein complex as disclosed herein. Each of the two or more recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two or more expression vectors are introduced into the same host cells, the antibody or the multi-specific protein complex produced therein can be recovered from the host cells or from the culture medium. If necessary, the polypeptide chains can be recovered from the host cells or from the culture medium and then incubated under suitable conditions allowing for formation of the antibody or the protein complex. When the two or more expression vectors are introduced into different host cells, each of them can be recovered from the corresponding host cells or from the corresponding culture media. The two or more polypeptide chains can then be incubated under suitable conditions for formation of the antibody or the multi-specific protein complex.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-nectin4 antibody, or the nucleic acids encoding the multiple polypeptides of a multi-specific protein complex as described herein, vectors (e.g., expression vectors) containing such; and host cells comprising the vectors are within the scope of the present disclosure.

### III. Applications of Anti-Nectin4 Antibodies or the Multi-Specific Protein Complexes Comprising Such

Any of the anti-nectin4 antibodies and multi-specific protein complexes disclosed herein can be used for therapeutic, diagnostic, and/or research purposes, all of which are within the scope of the present disclosure.

### Pharmaceutical Compositions

The antibodies and the multi-specific protein complexes, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 m, particularly 0.1 and 0.5 m, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Therapeutic Applications

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered close inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder characterized by carrying nectin4⁺ disease cells. Examples of such target diseases/disorders include cancer, e.g., a cancer comprising nectin4⁺ cancer cells. Examples include, but are not limited to, breast cancer, bladder cancer, ovary cancer, cervical cancer, pancreatic cancer, lung cancer, or head and neck cancer.

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody or protein complex achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe close according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies or multi-specific protein complexes comprising such as described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody or protein complex used) can vary over time.

In some embodiments, for an adult patient of normal weight, closes ranging from about 0.3 to 5.00 mg/kg may be administered. In some examples, the dosage of the anti-nectin4 antibody or the multi-specific protein complex comprising such as described herein can be 10 mg/kg. The particular dosage regimen, i.e., close, timing and repetition, will depend on the particular individual and that individual's medical history, as well as the properties of the individual agents (such as the half-life of the agent, and other considerations well known in the art).

For the purpose of the present disclosure, the appropriate dosage of an antibody or a protein complex comprising such as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody or protein complex comprising such may be essentially continuous over a preselected period of time or may be in a series of spaced close, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., close, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

### Diagnostic Applications

Any of the anti-nectin4 antibodies disclosed here may be used for detecting and quantifying nectin4 levels or nectin⁺ cell levels in a biological sample using a conventional method, for example, any immunohistological method known to those of skill in the art (see, e.g., Jalkanen, et al., J. Cell. Biol. 101:976-985 (1985); Jalkanen et al., J. Cell Biol. 105:3087-3096 (1987)). Other antibody-based methods useful for detecting nectin4 expression include immunoassays, such as the enzyme linked immunosorbent assay (ELISA), immunoprecipitation, or Western blotting. Suitable assays are described in more detail elsewhere herein.

The term “biological sample” means any biological sample obtained from an individual, cell line, tissue culture, or other source of cells potentially expressing nectin4 Methods for obtaining tissue biopsies and body fluids from mammals are well known in the art.

To perform the method disclosed herein, any of the anti-nectin4 antibodies as disclosed herein can be brought in contact with a sample suspected of containing a target antigen as disclosed herein, for example, a human nectin4 protein or a nectin4⁺ cell. In general, the term “contacting” or “in contact” refers to an exposure of the anti-nectin4 antibody disclosed herein with the sample suspected of containing the target antigen for a suitable period under suitable conditions sufficient for the formation of a complex between the anti-nectin4 antibody and the target antigen in the sample, if any. The antibody-antigen complex thus formed, if any, can be determined via a routine approach. Detection of such an antibody-antigen complex after the incubation is indicative of the presence of the target antigen in the sample. When needed, the amount of the antibody-antigen complex can be quantified, which is indicative of the level of the target antigen in the sample.

In some examples, the anti-nectin4 antibodies as described herein can be conjugated to a detectable label, which can be any agent capable of releasing a detectable signal directly or indirectly. The presence of such a detectable signal or intensity of the signal is indicative of presence or quantity of the target antigen in the sample. Alternatively, a secondary antibody specific to the anti-nectin4 antibody or specific to the target antigen may be used in the methods disclosed herein. For example, when the anti-nectin4 antibody used in the method is a full-length antibody, the secondary antibody may bind to the constant region of the anti-nectin4 antibody. In other instances, the secondary antibody may bind to an epitope of the target antigen that is different from the binding epitope of the anti-nectin4 antibody. Any of the secondary antibodies disclosed herein may be conjugated to a detectable label.

Any suitable detectable label known in the art can be used in the assay methods described herein. In some embodiments, a detectable label can be a label that directly releases a detectable signal. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

In some embodiments, the detectable label may be a molecule that releases a detectable signal indirectly, for example, via conversion of a reagent to a product that directly releases the detectable signal. In some examples, such a detectable label may be an enzyme (e.g., 0-galactosidase, HRP or AP) capable of producing a colored product from a colorless substrate.

### IV. Kits for Use in Treatment of Diseases

The present disclosure also provides kits for use in treating or alleviating a target disease, such as nectin4⁺ cancers as described herein. Such kits can include one or more containers comprising an anti-nectin4 antibody or multi-specific protein complex comprising such, e.g., any of those described herein. In some instances, the anti-nectin4 antibody or the protein complex comprising such may be co-used with a second therapeutic agent.

In some embodiments, the kit can comprise instructions for use in accordance with any of the methods described herein. The included instructions can comprise a description of administration of the anti-nectin4 antibody or protein complex comprising such, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-nectin4 antibody or protein complex comprising such generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-nectin4 antibody or protein complex comprising such as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Discovery of Anti-Nectin4 Antibody

This example describes isolation and characterization of anti-nectin4 antibodies.

(a) scFv mRNA Display Screening and Selection

In vitro mRNA display technology was applied for the identification of Nectin4 binders from natural human scFv libraries with a size of ˜10¹²⁻¹³. Briefly, DNA libraries containing fully human antibody heavy and light chain variable domains were first transcribed into mRNA libraries and then translated into mRNA-scFv fusion libraries by covalent coupling through a puromycin linker, similar to the reported procedure in US U.S. Pat. No. 6,258,558B1, the relevant disclosures of which are incorporated by reference for the subject matter and purpose referenced herein. The fusion libraries were first counter selected with human IgGs (negative proteins) to remove non-specific binders, followed by selection against recombinant Nectin4-Fc fusion protein. The resultant binders were captured on Protein G magnetic beads. To enrich scFvs having specific binding activity to cell surface Nectin4, antibodies were selected against a CHO cell line stably overexpressing Nectin4 to capture the Nectin4 binders, which were further enriched by PCR amplification with library specific primers. 5 rounds of selections were performed to generate a highly enriched Nectin4 binding pool for further screening.

### (b) Identification and Characterization of Anti-Nectin4 Antibodies

After 5 rounds of selections, the Nectin4 enriched scFv library was cloned into bacterial periplasmic expression vector pET22b and transformed into TOP 10 competent cells. A C-terminal flag and 6×His tag was fused to the scFv molecule for purification and assay detection purposes. Clones from TOP 10 cells were pooled and the miniprep DNA were prepared and subsequently transformed into bacterial Rosetta II strain for expression. Single clone was picked, grown and induced with 0.1 mM IPTG in 96 well plate. After 16-24 hours induction at 30° C., the supernatant was collected for assays to identify anti-Nectin4 antibodies.

An Nectin4 binding screening ELISA was developed for the identification of individual anti-Nectin4 ScFv antibodies. Briefly, 384 well plate was immobilized with human Fc, human Nectin4-Fc respectively, at final concentration of 2 ug/mL in 1×PBS in total volume of 25 uL per well. The plate was incubated overnight at 4° C. followed by blocking with 80 uL of superblock per well for 1 hour. 25 uL of supernatant was added to Fc and human Nectin4 immobilized wells and incubated for 1 hour with shaking. The Nectin4 binding was detected by adding 25 uL of anti-Flag HRP diluted at 1:5000 in 1×PBST. In between each step, the plate was washed 3 times with 1×PBST in a plate washer. The plate was then developed with 20 uL of TMB substrate for 5 mins and stopped by adding 20 uL of 2 N sulfuric acid. The plate was read at OD450 nm Biotek plate reader and the binding and selectivity was analyzed with Excel bar graph. Clones with Nectin4 target binding over human Fc>2-fold were subjected for DNA sequencing. The unique clones were produced and purified for further characterization.

(c) scFv Antibody Production in E. Coli

The specified anti-Nectin4 clone was picked from a glycerol stock plate and grown overnight into a 5 mL culture in a Thomson 24-well plate with a breathable membrane. This culture, and all subsequent cultures described below were grown at 37° C. and shaking at 225 RPM in Terrific Broth Complete plus 100 ug/mL carbenicillin and 34 ug/mL chloramphenicol, with 1:5,000 dilution of antifoam-204 also added, unless specified otherwise. This overnight starter culture was then used to inoculate the larger culture, 1:100 dilution of starter culture into the designated production culture and grown until OD600 was between 0.5-0.8. At this point, the culture was induced with a final concentration of IPTG at 0.1 mM and incubated over night at 30° C. The following day, the cultures were spun for 30 min at 5,000×g, to pellet the cells and then the supernatant was filter sterilized through a 0.2 um sterilizing PES membrane.

For purification, 3 uL GE Ni Sepharose Excel resin per 1 mL of filtered supernatant was used. Disposable 10 mL or 20 mL BioRad Econo-Pac columns were used. The resin was equilibrated with at least 20 column volume (CV) buffer A (1×PBS, pH7.4 with extra NaCl added to 500 mM). The filter sterilized supernatant was purified by gravity flow by either controlling the flow to 1 mL/min or was poured over two times, over same packed resin bed. The column was then washed with the following buffers: 10 CV buffer A, 20 CV buffer B (1×PBS, pH7.4 with extra NaCl to 500 mM, and 30 mM imidazole). The two Detox buffers were used to remove endotoxin as optional step if needed. For 250 mL expression culture purifications, antibody bound column was washed sequentially with 20 CV buffer C (1×PBS pH7.4 with extra NaCl to 500 mM, 1% Tx114), 20 CV buffer D (1×PBS pH7.4 with extra NaCl to 500 mM, 1% Tx100+0.2% TNBP) and 40 CV buffer E (1×PBS pH7.4 with extra NaCl to 500 mM). The protein was eluted with Eluting buffer F (1×PBS pH7.4 with extra NaCl to 500 mM, and 500 mM imidazole) in a total of six fractions (0.5 CV pre elute, 5×1 CV elute). Fractions were run on a Bradford assay (100 ul diluted Bradford solution+10 ul sample). Fractions with bright blue color were pooled. Protein concentration was measured by A280 extension coefficient. SDS-PAGE gel to analyze the purity of the purified antibodies. In some cases, Tm shift thermal stability assay was run to measure the thermal stability of the purified antibodies.

### (d) Assessing Binding of Scfv Antibodies to Nectin4 Via ELISA

An ELISA assay was developed to determine the EC₅₀ of anti-Nectin4 antibodies. Briefly, 384 well plate was immobilized with human Nectin4-Fc at final concentration of 2 ug/mL in 1×PBS in total volume of 25 uL per well. The plate was incubated overnight at 4° C. followed by blocking with 80 uL of superblock per well for 1 hour. Purified anti-Nectin4 scFvs were 2-fold serial titrated from 200 nM. 25 uL was added to human Nectin4 immobilized wells and incubated for 1 hour with shaking. The Nectin4 binding was detected by adding 25 uL of anti-Flag HRP diluted at 1:5000 in 1×PBST. In between each step, the plate was washed 3 times with 1×PBST in a plate washer. The plate was then developed with 20 uL of TMB substrate for 5 mins and stopped by adding 20 uL of 2 N sulfuric acid. The plate was read at OD450 nm Biotek plate reader and then plotted in Prism 8.1 software. EC₅₀ values were calculated and showed in Table 1 below.

TABLE 1
Binding Activity of Exemplary Anti-Nectin4
Antibodies to Nectin4 via ELISA

| Clones | Nectin4 Binding ELISA EC50 (nM) |
|---|---|
| 2020EP034-H09 | 11.84 |
| 2020EP034-B09 | 5.07 |
| 2020EP034-E01 | positive |
| 2020EP47-F02 | positive |
| 2021EP030-B10 | positive |
| 2021EP030-C11 | 290.6 |
| 2021EP030-D06 | 0.57 |
| 2021EP030-E10 | 26.59 |
| 2021EP030-F02 | 2.97 |
| 2021EP030-H06 | 6.87 |
| 2021EP029-C04 | 0.857 |
| 2021EP032-D10 | 8.76 |
| 2021EP032-E06 | 0.412 |

(e) Assessing Binding of scFv Antibodies to Nectin4 Via Surface Plasmon Resonance (SPR)

Kinetic analysis of anti-Nectin4 scFvs has been assessed by SPR technology with Biacore T200. The assay was run with Biacore T200 control software version 2.0. Anti-human Fc antibody was immobilized on flow cell 1 and 2 of CM5 sensor chip. For each cycle, 1 ug/mL of human Nectin4-Fc protein was captured for 60 seconds at flow rate of 10 ul/min on flow cell 2 in 1×HBSP buffer on anti-hFc sensor chip. 2-fold serial diluted HIS tag purified anti-Nectin4 scFv was injected onto both reference flow cell 1 and Nectin4-Fc captured flow cell 2 for 150 seconds at flow rate of 30 ul/min followed by wash for 300 seconds. The flow cells were then regenerated with Antibody regeneration buffer (GE) for 30 seconds at flow rate of 30 ul/mins. 8 concentration points from 300-0 nM was assayed per anti-Nectin4 scFv in a 96 well plate. The kinetics of scFvs binding to Nectin4 protein was analyzed with Biacore T200 evaluation software version 3.0. The specific binding response unit was derived from subtraction of binding to reference flow cell 1 from Nectin4 captured flow cell 2. The Kₒₙ, K_{off} and K_{D} values were calculated for selected ScFv antibodies and showed in Table 2 below.

TABLE 2
Kinetic Analysis of Exemplary Anti-Nectin4 Antibodies

| Clones | Ka (1/ms) | Kd (1/s) | KD (M) |
|---|---|---|---|
| 2020EP034-H09 | NA | NA | NA |
| 2020EP034-B09 | 1.221E+5 | 1.797E−4 | 1.472E−9 |
| 2020EP034-E01 | 3.704E+4 | 4.058E−4 | 1.095E−8 |
| 2020EP47-F02 | NA | NA | NA |
| 2021EP023-D06 | 4.797E+5 | 1.953E−4 | 4.071E−10 |
| 2021EP023-H06 | 4.198E+4 | 2.617E−4 | 6.234E−9 |
| 2021EP030-F02 | 1.160E+5 | 2.146E−4 | 1.850E−9 |
| 2021EP030-C11 | 4.714E+3 | 2.618E−4 | 5.554E−8 |
| 2021EP029-C04 | NA | NA | NA |
| 2021EP032-D10 | NA | NA | NA |
| 2021EP032-E06 | NA | NA | NA |

(f) Assessing Binding of scFv Antibodies to Cell Surface Nectin4 Via Fluorescence Activated Cell Sorting (FACS)

CHO cells (ATCC) were transfected with a construct encoding the full-length human Nectin4 with C-terminal flag and Myc tags in pCMV6-Entry vector. G418 drug selection process yielded a polyclonal, drug resistant pool of Nectin4 target-expressing cells. In parallel, the empty vector transfected parental line was generated as a negative control. The Nectin4 target-expressing cells were sorted by FACS to yield a Nectin4 target expressing polyclonal pool. The pool was expanded under G418 drug selection. Single cell sorting then was performed followed by further drug selection to form clonal cell lines. The clonal lines were screened for Nectin4 expression by FACS. The high expression Nectin4 cell line was then used for screening and assays.

To determine whether anti-Nectin4 scFvs bind to Nectin4 expressing cells, 200 nM of purified anti-Nectin4 scFv antibodies were diluted in full medium and incubated with Nectin4/CHO and CHO cells in 96 wells plate on ice for 1 hour. Cells were spun down at 1200 rpm for 5 minutes at 4° C. to remove primary antibodies. Cells were then washed once with 200 uL of full medium per well. Samples were detected with premixed anti-His Biotin Streptavidin Alexa fluor 647 by adding 100 uL of diluted secondary antibody and incubated at 4° C. for 30 minutes in the dark. Samples were spun down at 1200 rpm for 5 minutes at 4° C. and washed twice with 200 uL of 1×PBS per well. Reconstituted samples in 200 uL of 1×PBS and read on Attune NxT cytometer. Analysis was done by Attune NxT software plotting the overlay histogram of anti-Nectin4 scFvs binding onto both negative and target cell lines. EC₅₀ values of exemplary scFv antibodies for binding to cell surface Nectin4 were calculated and showed in Table 3 below.

TABLE 3
Binding Activity to Cell Surface Nectin4

| Clones | Nectin4/CHO Binding FACS EC50 (nM) |
|---|---|
| 2020EP034-H09 | weak |
| 2020EP034-B09 | 0.36 |
| 2020EP034-E01 | >50 |
| 2020EP47-F02 | 4.51 |
| 2021EP030-B10 | 24.19 |
| 2021EP030-C11 | 13.02 |
| 2021EP030-D06 | NA |
| 2021EP030-E10 | 7.17 |
| 2021EP030-F02 | 0.194 |
| 2021EP030-H06 | 1.49 |
| 2021EP029-C04 | 0.001 |
| 2021EP032-D10 | 0.29 |
| 2021EP032-E06 | <0.001 |

### Example 2: Characterization of Anti-Nectin4 Antibodies in Igg Format

The variable V_{H} and V_{L} regions of the scFv antibodies isolated in Example 1 were fused to the constant frame sequence of human heavy chain IgG1 backbone and light chain lambda backbone, respectively to generate anti-Nectin4 IgG antibody.

### (a) Binding Activity to Nectin4

An ELISA assay was developed to determine the EC₅₀ of exemplary anti-Nectin4 IgG antibodies. Briefly, 384 well plate was immobilized with human Nectin4-HIS tagged recombinant protein at final concentration of 2 ug/mL in 1×PBS in total volume of 25 uL per well. The plate was incubated overnight at 4° C. followed by blocking with 80 uL of superblock per well for 1 hour. Titration of purified anti-Nectin4 IgG starting at 200 nM 2-fold serial dilution, 25 uL was added to human Nectin4 immobilized wells and incubated for 1 hour with shaking. The Nectin4 binding was detected by adding 25 uL of anti-hFc HRP diluted at 1:5000 in 1×PBST. In between each step, the plate was washed 3 times with 1×PBST in a plate washer. The plate was then developed with 20 ul of TMB substrate for 5 mins and stopped by adding 20 ul of 2N sulfuric acid. The plate was read at OD450 nm Biotek plate reader and then plotted in Prism 8.1 software. Table 4 bellowed showed the EC50 values of the tested IgG antibodies to human, mouse, and monkey nectin4 proteins via ELISA.

TABLE 4
Binding Activity to Nectin4 of Various Species via ELISA

| | IgG | Nectin4 Binding ELISA (nM) | | |
|---|---|---|---|---|
| Antibody | Construct pairs | Human | Mouse | Monkey |
| 2020EP034-H09 | EP298/EP288 | NA | NA | NA |
| 2020EP034-B09 | EP299/EP289 | 0.018 | 0.017 | 0.019 |
| 2020EP034-E01 | EP296/EP286 | 0.035 | 0.037 | 0.043 |
| 2020EP47-F02 | EP544/EP545 | 0.119 | NA | NA |

Kinetic analysis of anti-Nectin4 IgG has been assessed by the SPR technology with Biacore T200. The assay was run with Biacore T200 control software version 2.0. For each cycle, 1 ug/mL of anti-hNectin4-IgG was captured for 60 seconds at flow rate of 10 ul/min on flow cell 2 in 1×HBSP buffer on Protein A sensor chip. 2-fold serial hNectin4-HIS tagged protein was injected onto both reference flow cell 1 and anti-Nectin4 IgG captured flow cell 2 for 150 seconds at flow rate of 30 ul/mins followed by wash for 300 seconds. The flow cells were then regenerated with Glycine pH2 for 60 seconds at flow rate of 30 ul/mins. 8 concentration points from 100-0 nM was assayed per anti-Nectin4 IgG in a 96 well plate. The kinetics of Anti-Nectin4 IgG binding to Nectin4 protein was analyzed with Biacore T200 evaluation software version 3.0. The specific binding response unit was derived from subtraction of binding to reference flow cell 1 from antibody captured flow cell 2. Table 5 below showed the binding kinetics of the anti-Nectin4 IgG antibody by SPR.

TABLE 5
Kinetic Analysis of Exemplary Anti-Nectin4 IgG Antibodies

| Antibody | Construct pairs | Kon | Koff | KD (nM) |
|---|---|---|---|---|
| 2020EP034-H09 | EP298/EP288 | NA | NA | NA |
| 2020EP034-B09 | EP299/EP289 | 1.221E+5 | 1.797E−4 | 1.472E−9 |
| 2020EP034-E01 | EP296/EP286 | 3.704E+4 | 4.058E−4 | 4.058E−4 |
| 2020EP47-F02 | EP544/EP545 | NA | NA | NA |

### (b) Binding Activity to Cell Surface Nectin4

200 nM of purified anti-Nectin4 IgG antibodies were diluted in full medium and incubated with Nectin4/CHO and CHO cells in 96 wells plate on ice for 1 hour. Cells were spun down at 1200 rpm for 5 minutes at 4° C. to remove primary antibodies. Cells were then washed once with 200 uL of full medium per well. Samples were detected with anti-hFc Alexa fluor 647 by adding 100 uL of diluted secondary antibody and incubated at 4° C. for 30 minutes in the dark. Samples were spun down at 1200 rpm for 5 minutes at 4° C. and washed twice with 200 uL of 1×PBS per well. Reconstituted samples in 200 uL of 1×PBS and read on Attune NxT cytometer. Analysis was done by Attune NxT software plotting the overlay histogram of BCMA proteins binding onto both negative and target cell lines. Table 6 below listed the EC₅₀ values of the anti-Nectin4 IgG antibodies for binding to Nectin4-expressing CHO cells.

TABLE 6
Binding of Anti-Nectin4 IgG Antibodies to Cell Surface Nectin4

| Antibody | Construct pairs | EC50 (nM) via FACS |
|---|---|---|
| 2020EP034-H09 | EP298/EP288 | NA |
| 2020EP034-B09 | EP299/EP289 | 0.0062 |
| 2020EP034-E01 | EP296/EP286 | 0.017 |
| 2020EP47-F02 | EP544/EP545 | 0.125* |

### (c) Binding of Anti-Nectin4 IgG Antibodies to Nectin4-Expressing Cells

Nectin4 expressing cell lines were characterized using Quantum Simply Cellular Kit (Bangs Laboratories 18102405-1). CHOK1, CHOK1/Nectin4, A549, HT29, HT1376, HCC1500, T47D were seeded in a 96 wells plate @37° C. for 1 hour. Cells were washed once with 100 uL 1×PBS and stained with 100 uL/well Zombie Fixable Viability Dyes for 30 mins @37° C. Samples were spun down at 1200 rpm for 5 minutes at 4° C. and washed twice with 100 uL of flow buffer. Samples were blocked by 1.25 uL Human TrueStain FcX™ (Fc Receptor Blocking Solution) diluted with 23.75 uL flow buffer for 10 minutes at RT. Human Nectin-4 PE-conjugated antibody (R&D system FAB2659P) was diluted in 1:100 with 1×PBS. Samples were then stained with 50 uL/well Human Nectin-4 PE-conjugated antibody and incubated for 1 hour at 4° C. in dark. For generating the standard curve, Human Nectin-4 PE conjugated antibody was incubated with 1 drop of Quantum Simply Cellular for 1 hour at 4° C. in dark. Samples were spun down at 1200 rpm for 5 minutes at 4° C. and washed twice with 200 uL of flow buffer per well. The samples were reconstituted in 200 uL of flow buffer and read on Attune NxT cytometer. Analysis was done by Attune NxT software. Using the standard curve generated by the Quantum Simply Cellular Kit, the number of receptors for each cells line was calculated based on the MFI of each cell type stained with Human Nectin-4 PE-conjugated antibody. The binding activities of single concentration of EP458/EP378/EP289 to those cells were assessed by FACS analysis. (−) indicated no binding. +++, ++ and + represent the strong, medium and low binding activities, respectively. See Table 7 below.

TABLE 7
Number of Nectin4 Receptor on Various Cell Lines

| | | | # of | EP458/EP378/ |
|---|---|---|---|---|
| Cell | Type | MFI | Receptors | EP289 Binding |
| A549 | Lung cancer | 339 | −23727 | − |
| CHOK | Neg. Ctl. | 206 | −24661 | − |
| CHOK/Nectin4 | Pos. Ctl. | 43811 | 281339 | +++ |
| T47D | Breast cancer | 17527 | 96889 | +++ |
| HT1376 | Bladder cancer | 12804 | 63746 | ++ |
| HCC1500 | Breast cancer | 6767 | 21381 | ++ |
| HT-29 | Colon Cancer | 3923 | 1423 | + |

### (d) ADCC Activity of Anti-Nectin4 IgG Antibodies

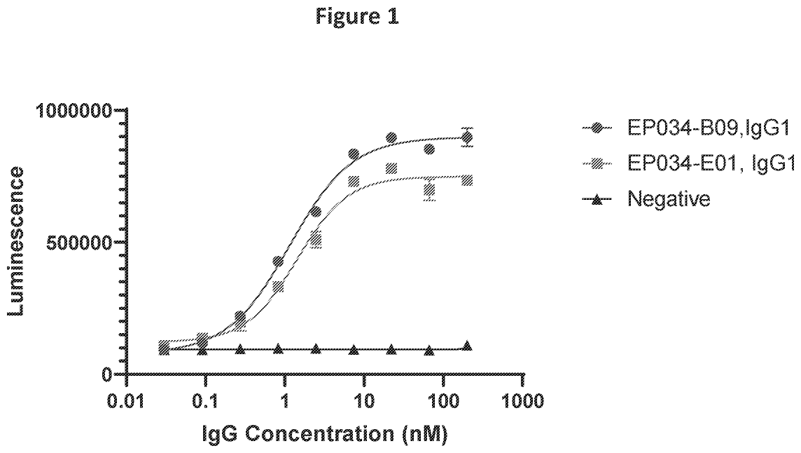

The ADCC activities of exemplary anti-Nectin4 antibody were tested with Promega ADCC Bioreporter assay kit. Briefly, 30,000 Nectin4/CHO target cells were plated on white bottom flat 96 well assay plate and incubated at 37° C. overnight. Following manufacture's protocol, antibodies were 3-fold serial diluted from 200 nM in ADCC assay buffer. Supernatant from target cells was removed. 25 uL of ADCC assay buffer mixed with 25 uL of antibody dilution was added to each well of cells. Cells were incubated at room temperature for 1 hour before effector cells added. Effector cells were thawed following manufacture's protocol and 25 uL of effector cells was plated to each target cells/antibody mixture. The plate was incubated at 37C for 16 hours. Next day, samples were equilibrated at room temperature for 30 minutes and then 75 uL of room temperature Bio-glow reagent was added and incubated at room temperature shaking for 30 minutes in dark. Bio-glow reagent was prepared according to the manufacturing protocol. The plate was read with luminescence on Biotek Neo2 plate reader and data was graphed on Prism 8.0. FIG. 1 shows the ADCC activities of anti-Nectin4 monoclonal antibodies to Nectin4/CHO cells. The EC₅₀ values of EP034-B09 and EP034-E01 are 1.16 nM and 1.43 nM, respectively.

### Example 3: Construction of Multi-Specific Antibodies

The scFv sequences of mouse and humanized anti-CD3 OKT3 antibody, and the humanized SP34 antibody were chosen to generate anti-Nectin4 and anti-CD3 bispecific antibody. To generate monovalent anti-Nectin4/anti-CD3 bispecific antibody, the respective sequence of anti-CD3 antibody was directly fused to the constant CH2 and CH3 region of human IgG1. The S354C, T366W and K409A mutations (Wei et al., Oncotarget, 2017; Xu et al., mAbs, 2015) were introduced to make the chain as a knob molecule. The S354C, Y349C, T366S, L368A, F405K, Y407V mutations (Wei et al, Oncotarget, 2017; Xu et al., mAbs, 2015) were introduced to the heavy chain of anti-Nectin4 antibody to generate a hole molecule. To generate bivalent anti-Nectin4/anti-CD3 bispecific antibody, the S354C, T366W and K409A mutations were introduced to the heavy chain of anti-Nectin4 antibody as a knob molecule.

The N-terminus of the ScFv sequence of anti-CD3 was linked to the C-terminus of the constant CH1 with a (G4S)₂ linker, and its C-terminus was directly linked to the N-terminus of the constant hinge of the anti-Nectin4 heavy chain. The S354C, Y349C, T366S, L368A, F405K, Y407V mutations were introduced to such molecule to generate a hole molecule. In some cases, the extracellular domain of the ligands of T cell stimulating receptors such as ICOS, 4-1BB, CD80, CD86 were fused to anti-CD3 antibody to generate a trispecific antibody. In another case, the IL2 molecule was fused to the C-terminus of above-mentioned hole molecules to create either anti-Nectin4 and IL2 fusion bispecific or monovalent or bivalent anti-Nectin4/anti-CD3 and IL2 fusion trispecific antibody. L234A, L235A and P329G mutations in both the knob and hole molecules were introduced to eliminate complement binding and Fc-γ dependent antibody-dependent cell-mediated cytotoxity (ADCC) effects (Lo et al., JBC 2017).

In some cases, the selected amino acids in the HCDR2 or HCDR3 of the anti-CD3 scFv within the bispecific format were mutated to the alanine residues in order to further fine tune the binding activities of the scFv to CD3. In particular, the EP500 clone was used as a template to generate EP695, EP696 and EP697 respectively.

The DNA encoding the entire above designed sequences were then synthesized with codon optimized for mammalian cell expression, and subcloned to pCDNA3.4 (Invitrogen). FIGS. 2A-2E show exemplary designs of bispecific antibodies, which optionally comprises a cytokine moiety.

### Example 4: Characterization of Anti-Nectin4/CD3 Bispecific Antibodies

### (a) ELISA Analysis of Binding Activity to Nectin4

An ELISA assay was developed to determine the EC₅₀ of anti-Nectin4/CD3 bispecific antibodies. Briefly, 384 well plate was immobilized with human CD3E or Nectin4 HIS tagged protein at final concentration of 2 ug/mL in 1×PBS in total volume of 25 uL per well. The plate was incubated overnight at 4° C. followed by blocking with 80 uL of superblock per well for 1 hour. Purified anti-Nectin4/CD3 was 3-fold serial titrated from 100 nM. 25 uL was added to human Nectin4/CD3 immobilized wells and incubated for 1 hour with shaking. The CD3E or Nectin4 binding was detected by adding 25 uL of anti-Human HRP diluted at 1:10000 in 1×PBST. In between each step, the plate was washed 3 times with 1λPBST in a plate washer. The plate was then developed with 20 ul of TMB substrate for 5 mins and stopped by adding 20 ul of 2N sulfuric acid. The plate was read at OD450 nm Biotek plate reader and then plotted in Prism 8.1 software to calculate EC50. Table 8 below show the binding activities of the bispecific antibodies to Nectin4 and CD3E via ELISA.

TABLE 8
Binding Activity to Nectin4 and CD3ζ

| | EC50 for Nectin4 | EC50 for CD3E |
|---|---|---|
| Antibodies | Binding (nM) | binding (nM) |
| EP369/EP370/EP289 | 0.019 | 0.005 |
| EP456/EP370/EP289 | 0.014 | weak |
| EP457/EP378/EP289 | 0.033 | weak |
| EP458/EP378/EP289 | 0.21 | 0.011 |
| EP499/EP378/EP289 | 0.025 | NA |
| EP500/EP378/EP289 | 0.022 | NA |

### (b) Binding to Cell Surface Antigens Via FACS

The binding activities of the bispecific antibodies to Nectin4 has been assessed by FACS. Nectin4/CHO Nectin4 cells were plated at 100,000 cells/well in a 96-well plate in 50 μL of full media. Cells were rested 1 hour at 37° C. Anti-Nectin4/CD3 was 3-fold serial titrated from 50 nM. 50 μL was added to the cells and incubated for 1 hours with shaking. Samples were washed one time with flow buffer. The samples were incubated with secondary Ab 100 uL Alexa Fluor 647 Goat Anti-Human IgG (Jackson ImmunoResearch 109-606-170) diluted at 1:1000 in full media for 1 hour at 4° C. Samples were washed once with flow buffer. Samples were stained with 100 uL Zombie Fixable Viability Dyes (1:800 dilution) per well and incubated in dark at room temperature for 30 mins. Samples were spun down at 1200 rpm for 5 minutes and washed once with 200 uL flow buffer. Reconstituted samples in 200 uL of flow buffer and read on Attune NxT cytometer. EC₅₀ values for Nectin4 Binding were determined using Prism software. Similar method was used to determine the binding activities of antibodies to cancer cell line T47D and HT1376.

The engagement of the antibodies to CD3 has been assessed by FACS. Jurkat cells were plated at 100,000 cells/well in a 96-well plate with 50 μL of full media. Anti-Nectin4/CD3 antibodies were 3-fold serial titrated from 20 nM. 50 μL was added to the cells and incubated for 1 hours at room temperature with shaking. Samples were washed one time with flow buffer. The samples were incubated with 100 uL Alexa Fluor® 647 Goat Anti-Human IgG (Jackson ImmunoResearch 109-606-170) diluted at 1:1000 in full media for 1 hour at 4° C. Samples were washed once with flow buffer. Samples were stained with 100 uL Zombie Fixable Viability Dyes at 1:800 dilution per well and incubated in dark at room temperature for 30 mins. Samples were spun down at 1300 rpm for 5 minutes and washed once with 200 uL flow buffer. Reconstituted samples in 200 uL of flow buffer and read on Attune NxT cytometer. EC50 values for CD3 binding were calculated using Prism 8.1 software.

Table 9 below shows the binding activity of the exemplary bispecific antibodies to cell surface nectin4 and CD3.

TABLE 9
Binding Activity to Cell Surface Antigens

| | | T47D | HT1376 | Jurkat CD3 |
|---|---|---|---|---|
| | CHO/Nectin4 | EC50 | EC50 | binding FACS |
| Antibodies | EC50 (nM) | (nM) | (nM) | (nM) |
| EP369/370/289 | 0.146 | NA | NA | 0.169 |
| EP456/370/289 | 0.253 | NA | NA | 1.03 |
| EP457/378/289 | 0.222 | 0.057 | 0.099 | 45.3 |
| EP458/378/289 | 0.146 | 0.044 | 0.140 | 4.7 |
| EP499/378/289 | 0.235 | NA | NA | NA |
| EP500/378/289 | 0.234 | NA | NA | NA |

### (c) Binding Kinetics by SPR

Kinetic analysis of exemplary anti-Nectin4/CD3 bispecific antibodies to CD3E and Nectin4 has been assessed by SPR technology with Biacore T200. The assay was run with Biacore T200 control software version 2.0. For each cycle, 1 ug/mL of anti-hNectin4/CD3 antibody was captured for 60 seconds at flow rate of 10 ul/min on flow cell 2 in 1×HBSP buffer on Protein A sensor chip. 2-fold serial diluted CD3E-HIS or hNectin4-HIS tagged protein was injected onto both reference flow cell 1 and anti-Nectin4/CD3 bispecific captured flow cell 2 for 150 seconds at flow rate of 30 ul/mins followed by wash for 300 seconds. The flow cells were then regenerated with Glycine pH2 for 60 seconds at flow rate of 30 ul/mins. 8 concentration points from 100 nM-0 nM (CD3E-HIS) or 300 nM-0 nM (Nectin4-HIS) was assayed per anti-Nectin4 IgG in a 96 well plate. The kinetics of Anti-Nectin4/CD3 bispecific binding to CD3E and Nectin4 proteins was analyzed with Biacore T200 evaluation software version 3.0. The specific binding response unit was derived from subtraction of binding to reference flow cell 1 from antibody captured flow cell 2. Table 10 below listed the kinetics results. ND means not determined. NA means data not available.

TABLE 10
Binding Kinetics of Exemplary Anti-Nectin4/CD3 Bispecific Antibodies

| | Nectin4 Binding SPR | | | CD3E Binding SPR | | |
|---|---|---|---|---|---|---|
| Antibody | ka (1/Ms) | kd (1/s) | KD (M) | ka (1/Ms) | kd (1/s) | KD (M) |
| EP369/EP370/EP289 | 9.875E+4 | 1.202E−4 | 1.217E−9 | 2.647E+5 | 5.900E−4 | 2.229E−9 |
| EP456/EP370/EP289 | 9.736E+4 | 4.118E−4 | 4.230E−9 | ND | ND | ND |
| EP457/EP378/EP289 | 9.803E+4 | 1.443E−4 | 1.472E−9 | ND | ND | ND |
| EP458/EP378/EP289 | 1.287E+5 | 4.716E−4 | 3.665E−9 | 2.268E+5 | 7.385E−4 | 3.257E−9 |
| EP499/EP378/EP289 | NA | NA | NA | NA | NA | NA |
| EP500/EP378/EP289 | NA | NA | NA | NA | NA | NA |

### (d) Internalization of Anti-Nectin4 IgG Antibodies

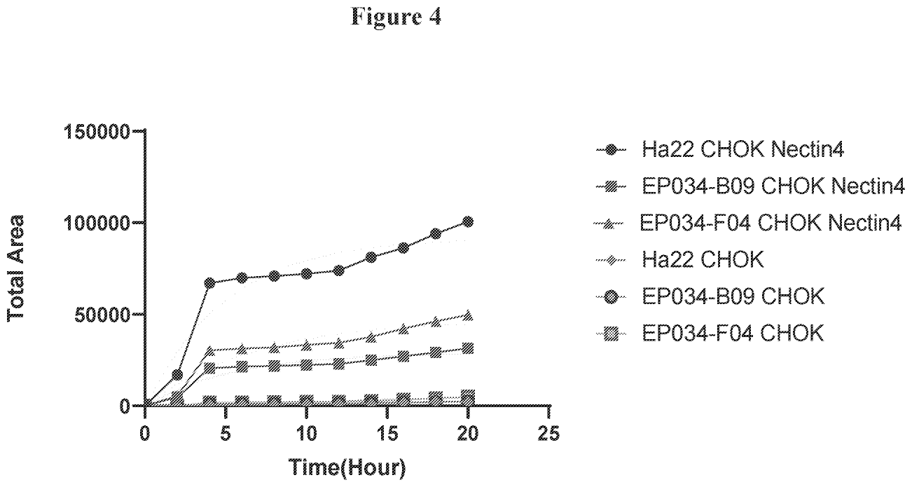

An internalization assay was performed to observe the internalization of the anti-nectin4 antibodies. Briefly, CHOK1/Nectin4 and CHOK1 cells were place in a 96-well plate at 5,000 cells in 50 uL cell culture medium per well. 200 uL Zenon™ pHrodo™ iFL Red-labeled Fab fragments (Invitrogen, Z25612) was diluted with 2.3 mL full media to make 4×Zenon working solution. Anti-Nectin4/CD3 antibody was diluted with full media to make 4× antibody working solution with the final concentration of 4.5 nM. 25 uL of 4× Zenon working solution was incubated with 25 uL of 4× antibody working solution for 5 minutes at room temperature. 50 uL Zenon pHrodo labeled antibody was then added to each well of the 96-well plate with cells. The samples were spun down at 500×g for 5 minutes and placed in Cytation 5 Cell Imaging Multi-Mode Reader. The anti-Nectin4 HA22 antibody was used as reference. Images were taken every 4 hours and confluence of the images were measured to analyze. FIG. 4 shows the internalization activities of anti-Nectin4 monoclonal antibodies to Nectin4/CHOK1 cells.

### (e) NFAT Reporter Assay

The activity of anti-Nectin4/CD3 bispecific antibodies in activating immune cells was examined in the Jurkat cell NFAT reporter assay (Promega, J1250) described above. The results are shown in Table 11 below.

TABLE 11
Immune Cell Activation Activity

| Antibody | NFAT reporter EC50 (nM) |
|---|---|
| EP369/370/289 | 0.0038 |
| EP456/370/289 | 0.1021 |
| EP457/378/289 | 0.169 |
| EP458/378/289 | 0.0004 |
| EP499/378/289 | 0.0014 |
| EP500/378/289 | NA |

### (f) In Vitro Cytotoxic T Lymphocyte Activities

In vitro cytotoxic T lymphocyte activity of anti-Nectin4/CD3 antibodies were performed by cancer cell killing assay. Pan T-cells were isolated from LRS clones of two separate donors using EasySep™ Human Pan T Cell Isolation Kit. Pan T-cells were plated at 25,000 cells/well in a black 96-well plate in 25 μL of phenol red free RPMI with 5% FBS. Anti-Nectin4/CD3 antibodies were 3-fold serial titrated from 50 nM. 50 μL of antibody was mixed with T-cells and incubated for 1 hours at 37° C. With the E:T ratio of 5:1, T47D RFP cells were added to samples at 5,000 cells/well in 25 μL phenol red free medium. The plate was spun at 500×g for 5 minutes. Cytation 5 Cell Imaging Multi-Mode Reader was used to take images every 4 hours and confluence of the images were measured to analyze the viability of the cells.

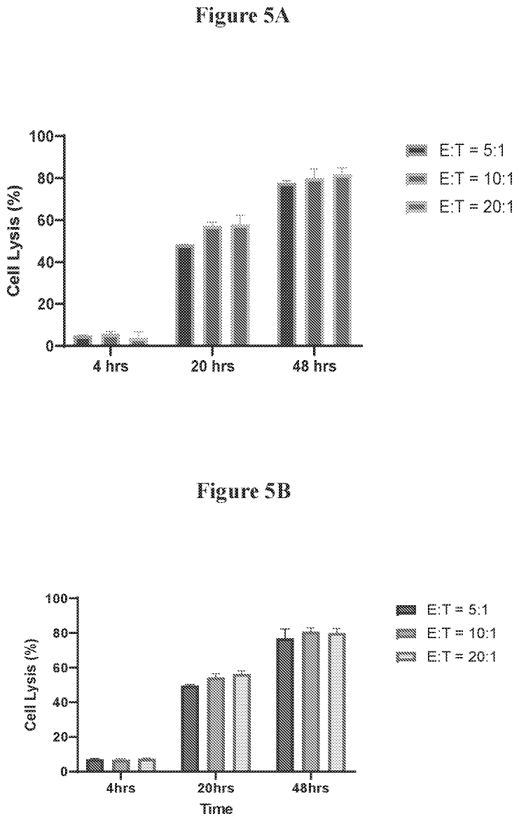

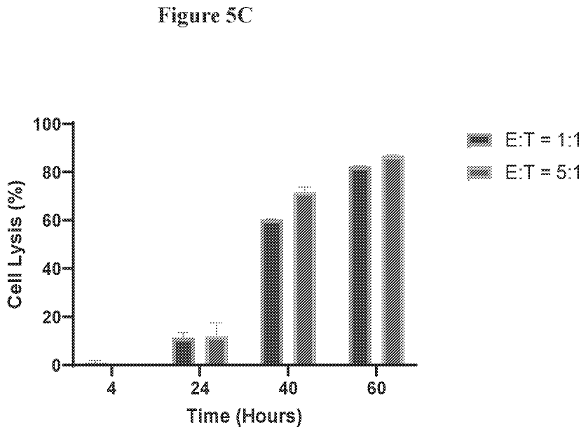

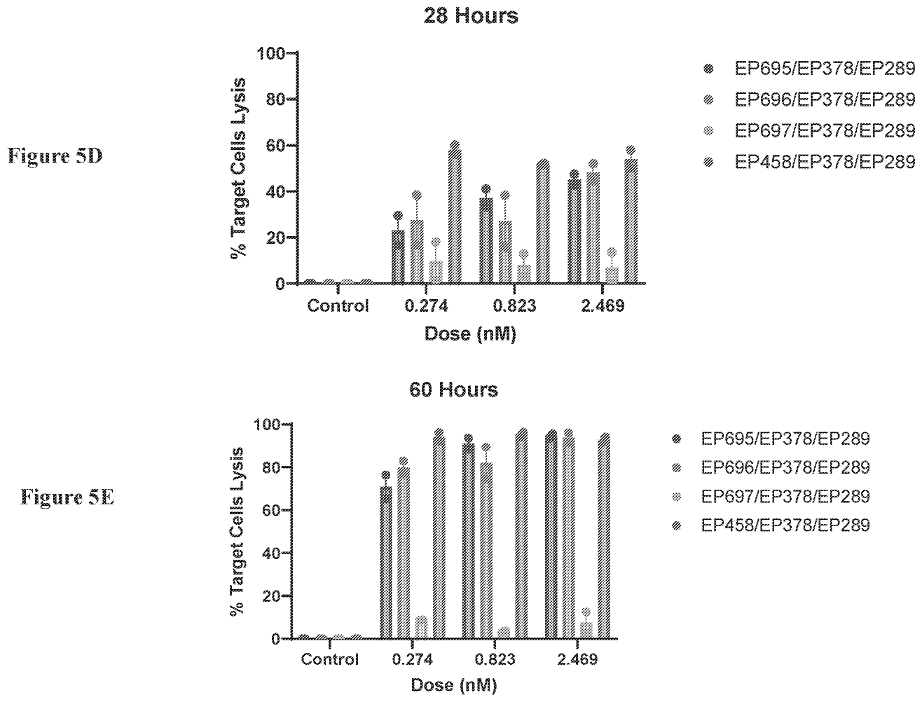

FIGS. 5A and 5B show the E:T ratio cancer cell killing activities of EP457/EP378/EP289 (25 nM) against Nectin4 positive MCF7 and T47D cell lines in PBMC. FIG. 5C shows the E:T ratio cancer cell killing activities of EP458/EP378/EP289 (2.5 nM) against Nectin4 positive T47D cell line in PBMC. FIG. 5D and FIG. 5E shows the E:T ratio (5:1) cancer cell killing activities of EP458/EP378/EP289, EP695/EP378/EP289, EP696/EP378/EP289 and EP697/EP378/EP289 at different concentration against Nectin4 positive T47D cell line in PBMC. EP697/EP378/EP289 lost its cancer killing activities consistent with its inability of CD3 cell binding. Whereas EP695/EP378/EP289 and EP696/EP378/EP289 retain their tumor killing activities, the activities are apparently lower than that of EP458/EP378/EP289.

The EC50 values are provided in Tables 12 and 13 below.

TABLE 12
EC50 Values in CTL Assay Against PBMC

| Antibody | EC₅₀ | |
|---|---|---|
| EP457/378/289 | 1.08 | nM |
| EP458/378/289 | 1.4 | pM |

TABLE 13
EC50 Values in CTL Assay Against T47D Cancer Cells

| Ab | EC50 (pM) |
|---|---|
| EP458/378/289, PBMC | 4.7 |
| EP458/378/289, T cell | 6.4 |
| PADCEV, PBMC | — |
| PADCEV, T cell | — |

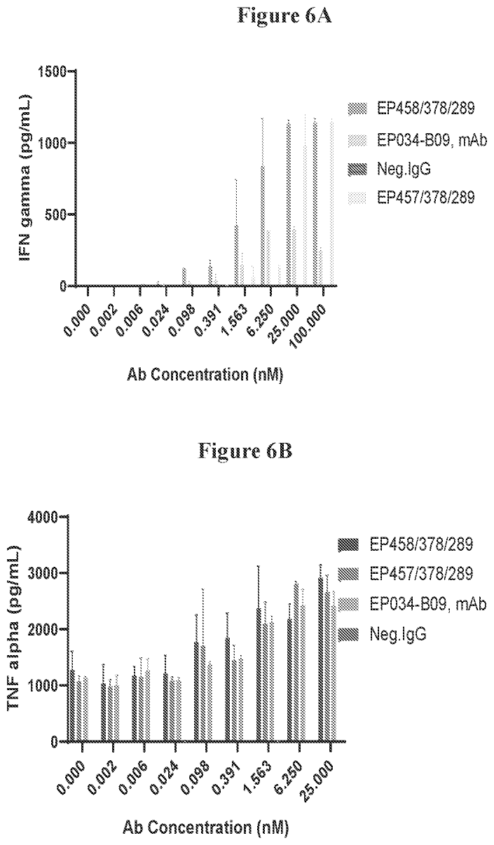

The bispecific antibodies were also found to induce cytokine release, such as IFNγ and TNFα release. FIGS. 6A and 6B.

### Example 5: Antibody Production

The anti-Nectin4 monoclonal antibody was expressed transiently in ExpiHEK293-F cells in free style system (Invitrogen) according to standard protocol with a ratio of the plasmid DNA of heavy chain and light chain of 1:2. The cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH2.5+150 mM NaCl and quickly neutralized with 20 mM citrate pH 5.0+300 mM NaCl. The antibody was then further purified by a Superdex 200 16/600 column. The monomeric peak fractions were pooled and concentrated. The final purified protein has endotoxin of lower than 10EU/mg and kept in 20 mM Histidine pH 6.0+150 mM NaCl.

The anti-Nectin4/anti-CD3 and anti-Nectin4/anti-CD3/IL-2 trispecific antibody production, the “knob” and “hole” constructs in respective IgG1 backbone formats were transfected to ExpiHEK293-F cells in free style system (Invitrogen) according to standard protocol. Cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH2.5+150 mM NaCl and quickly neutralized with 20 mM citrate pH 5.0+300 mM NaCl. The antibody was then further purified by a Superdex 200 Increase 16/600 column. The monomeric peak fractions were pooled and concentrated. The final purified protein has endotoxin of lower than 10 EU/mg and kept in 1×PBS buffer.

### Example 6: Antibody-Cytokine Protein Complexes and Characterization Thereof

Protein complexes comprising an anti-nectin4 moiety and an interleukin-2 (IL-2) moiety, or protein complexes comprising an anti-nectin4 moiety, an anti-CD3 moiety, and an IL-2 moiety were constructed following routine practice or disclosures provided herein. Exemplary formats of such protein complexes are provided in FIGS. 2A-2E and FIGS. 3A-3F. bioactivity of the protein complexes were explored as provided below.

### (a) Binding Activity Determined by ELISA

ELISA was performed to determine the EC₅₀ of anti-Nectin4/CD3/IL2 protein complexes. Briefly, 384 well plate was immobilized with human Nectin4-HIS tagged recombinant protein at final concentration of 2 ug/mL in 1×PBS in total volume of 25 uL per well. The plate was incubated overnight at 4° C. followed by blocking with 80 uL of superblock per well for 1 hour. Titration of purified anti-Nectin4/CD3/IL2 starting at 25 nM 3-fold serial dilution, 25 uL was added to human Nectin4 immobilized wells and incubated for 1 hour with shaking. The Nectin4 binding was detected by adding 25 uL of anti-hFc HRP diluted at 1:10000 in 1×PBST. In between each step, the plate was washed 3 times with 1λPBST in a plate washer. The plate was then developed with 20 ul of TMB substrate for 5 mins and stopped by adding 20 ul of 2N sulfuric acid. The plate was read at OD450 nm Biotek plate reader and then plotted in Prism 8.1 software. Similar SPR methods were applied to measure the binding of the protein complexes to Nectin4, CD3E and IL2 receptors. Table 14 below listed the EC₅₀ values of various protein complexes via ELISA.

TABLE 14
Binding Activity of Anti-Nectin4/CD3/IL2 Complexes

| Protein Complexes | EC₅₀ (nM) via ELISA |
|---|---|
| EP369/371/289 | 0.016 |
| EP369/379/289 | 0.042 |
| EP378/379/289 | 0.022 |
| EP034-B09, mAb | 0.061 |

### (b) Binding to Jurkat Cells by FACS

Similar methods of above mentioned were used to measure the binding activities of anti-Nectin4/CD3/IL-2 complexes to CHO/Nectin4 and Jurkat cells, respectively. Table 15 below showed the EC₅₀ values of the binding.

TABLE 15
Binding Activity to Jurkat Cells

| | Jurkat Cell Binding |
|---|---|
| Antibody | EC₅₀ (nM) |
| EP369/371/289 | 1.654 |
| EP369/379/289 | 1.04 |
| EP378/379/289 | — |
| EP458/378/280 | 4.37 |
| EP695/378/289 | weak |
| EP696/378/289 | weak |
| EP697/378/289 | ND |

* ND: no binding
“—”: not determined

### (c) Jurkat Cell Activation Determined by NFAT Reporter Assay

The target cells were cultured with Jurkat Cell Line containing a firefly luciferase gene under the control of the NFAT response element stably integrated into Jurkat cells. This cell line has also been validated for response to T cell activation through a variety of TCR activators. This reporter cell line has been designed to monitor T-cell activation as well as inhibition a 96 well plate at 15,000 cells/well in 96 well plate in 50 uL media. After 24 hrs. the Anti-Nectin/CD3 Ab is added to the well at a starting concentration of 1 nM with 3× dilution. After 1 hr of incubation add 30,000 cells/well Jurkat cells and leave the plate in the incubator at 37° C. for 24 hrs. Remove the plate from incubator and place them in room temperature for 15 minutes. 150 uL of Bio-Glo™ Reagent was add to each assay well. Samples were incubated at room temperature for 30 minutes. The plate was read at OD450 nm Biotek plate reader and then plotted in Prism 8.1 software to calculate EC50 values, which are provided in Table 16 below.

TABLE 16
Jurkat Cell Activation Activity

| Protein Complexes | EC50 (nM) |
|---|---|
| EP378/379/289 | — |
| EP369/371/289 | 0.009 |
| EP369/379/289 | 0.044 |

(d) p-STAT5 Activation

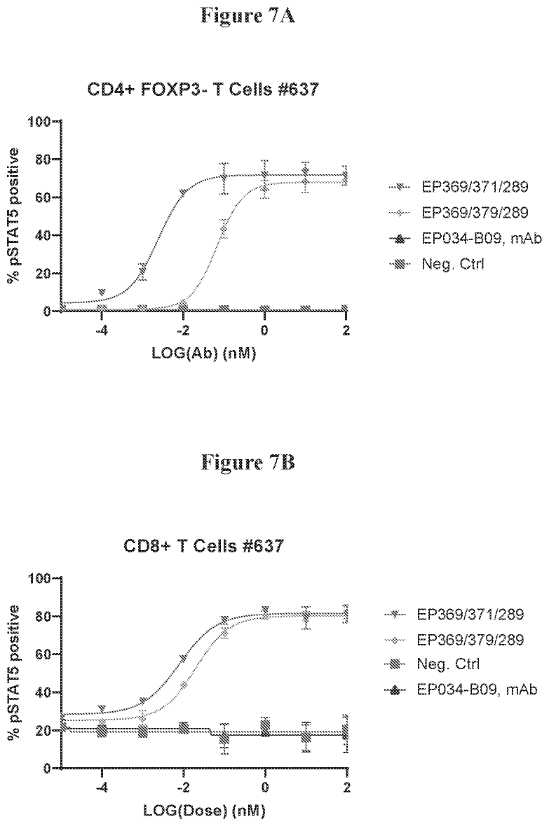

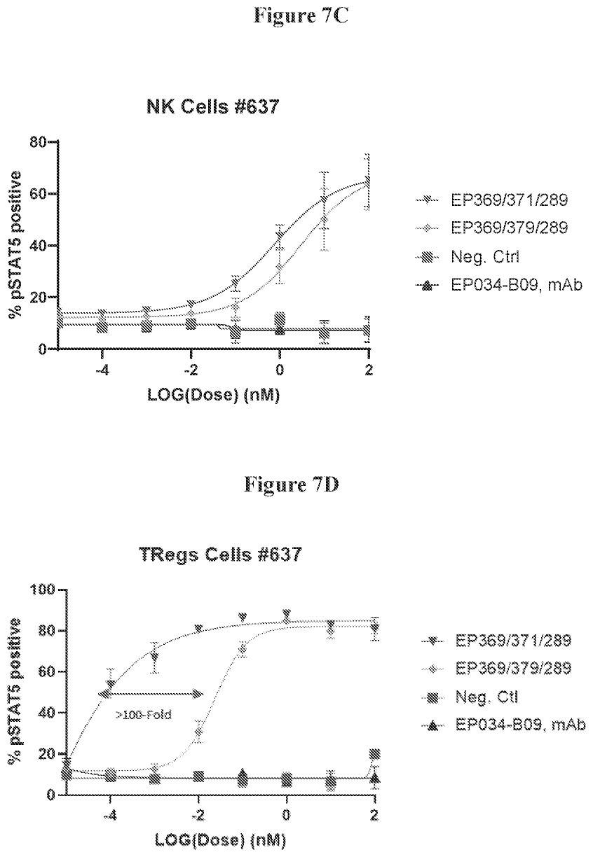

Human PBMCs were isolated from LRS cones of two separate donors and plated at 250,000 cells/well in a 96-well plate in 90 μL of media. Cells were rested 1 hr at 37° C. Cells were stimulated with human IL2-Fc wt and engineered IL2-Fc mutants at 10× concentration in 10 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56-CD4-CD8+. NK cells were defined as CD3-CD56+. T regulatory cells were defined as CD3+CD56-CD4+CD8-FOXP3+. The % of cells that were p-STAT5+ was determined and graphed versus each IL2 titration. The results are shown in FIGS. 7A-7D.

### (e) In Vitro Cytotoxic T Lymphocyte Activity

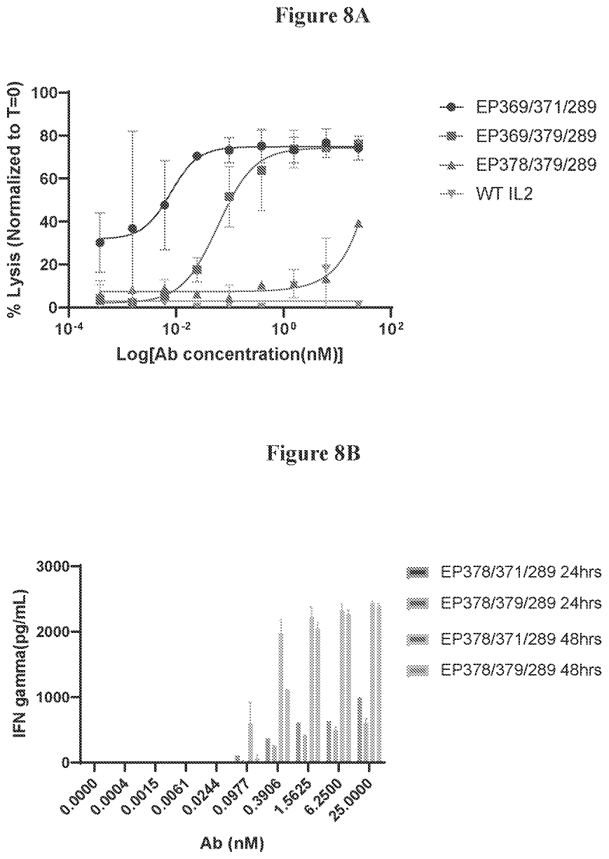

Same protocol as described above was used to determine the T-cell mediated cancer cell killing activities of tri-specific antibodies and anti-Nectin4/IL2 antibodies. FIG. 8A shows a dose dependent curve of cancer cell killing activities. Only anti-CD3 carrying protein complexes showed cancer cell killing activities. FIG. 8B shows IFN gamma release induced by the protein complexes as indicated.

### Example 7: In Vivo Tumor Growth Inhibition Activity of Anti-Nectin4/CD3 Antibody

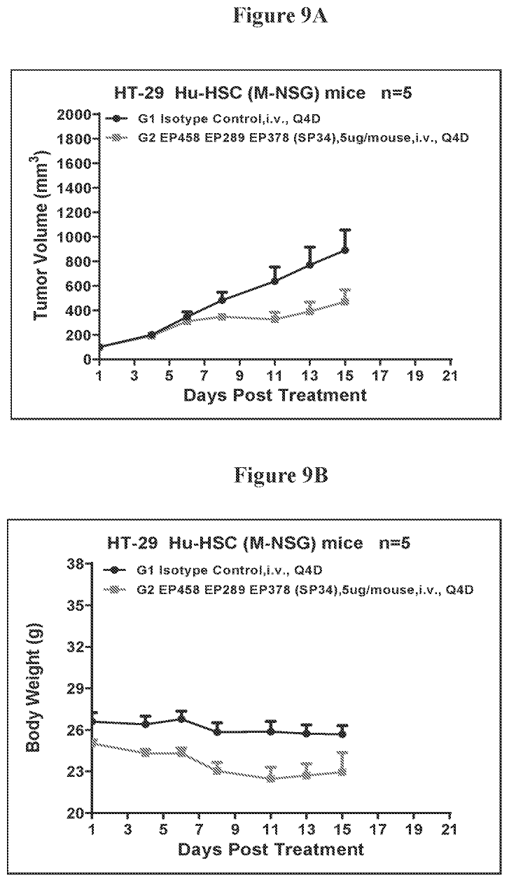

6-8-week-old, female Hu-HSC (M-NSG) mice were injected with 5×10⁶ cells HT-29 cells in 50% matrigel subcutaneously on their right flank region. When the average tumor size reached a volume of approximately 100 mm³ and when tumors were palpable, the experimental treatments were begun. The mice were treated with 5 ug human Anti-Nectin4 antibody EP458/EP378/EP289 or isotype control for 20 days. Tumor volume and body weight were measured twice/week. FIGS. 9A and 9B show more than 50% tumor growth inhibition of EP458/EP378/EP289 comparing to the vehicle group.

Sequence Table 1: Exemplary Anti-Nectin4 Antibodies

| | | | SEQ ID |
|---|---|---|---|
| Ab | | Amino Acid Sequences | NO |
| EP034- | HCDR1 | SYAIS | 1 |
| H09 | HCDR2 | GIIPIFGTANYAQKFQG | 2 |
| | HCDR3 | GSGTLNWFDP | 3 |
| | VH | QVQLVQSGGTFSSYAISWVRQAPGQGLEWMGGIIPIFGTANYAQKFQGR | 4 |
| | | VTITADESTSTAYMELSSLRSEDTAVYYCARGSGTLNWFDPWGQGTLVT | |
| | | VSS | |
| | H chain | METDTLLLWVLLLWVPGSTGQVQLVQSGGTFSSYAISWVRQAPGQGLEW | 5 |
| | (EP298) | MGGIIPIFGTANYAQKFQGRVTITADESTSTAYMELSSLRSEDTAVYYC | 167 |
| | | ARGSGTLNWFDPWGQGTLVTVSSASTKGPSVFPLAPSSKSTSGGTAALG | (no signal |
| | | CLVKDYFPEPVTVSWNSGALTSGVHTFPAVLQSSGLYSLSSVVTVPSSS | peptide) |
| | | LGTQTYICNVNHKPSNTKVDKKVEPKSCDKTHTCPPCPAPELLGGPSVF | |
| | | LFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKA | |
| | | KGQPREPQVYTLPPSRDELTKNQVSLTCLVKGFYPSDIAVEWESNGQPE | |
| | | NNYKTTPPVLDSDGSFFLYSKLTVDKSRWQQGNVFSCSVMHEALHNHYT | |
| | | QKSLSLSPGK | |
| | LCDR1 | TGTSSDIGGYNFVS | 6 |
| | LCDR2 | EVSKRPS | 7 |
| | LCDR3 | SSNSGDYADVV | 8 |
| | VL | QSVLTQPRSVSGSPGQSVTISCTGTSSDIGGYNFVSWYQQHPGKAPKLV | 9 |
| | | ITEVSKRPSGVPDRFSGSKSGNTASLTISGLQAEDEADYYCSSNSGDYA | |
| | | DVVFGGGTKLTVL | |
| | L chain | METDTLLLWVLLLWVPGSTGQSVLTQPRSVSGSPGQSVTISCTGTSSDI | 10 |
| | (EP288) | GGYNFVSWYQQHPGKAPKLVITEVSKRPSGVPDRFSGSKSGNTASLTIS | 168 |
| | | GLQAEDEADYYCSSNSGDYADVVFGGGTKLTVLGQPKANPTVTLFPPSS | (no signal |
| | | EELQANKATLVCLISDFYPGAVTVAWKADGSPVKAGVETTKPSKQSNNK | peptide) |
| | | YAASSYLSLTPEQWKSHRSYSCQVTHEGSTVEKTVAPTECS | |
| EP034- | HCDR1 | GYYMH | 11 |
| B09 | HCDR2 | RINPNSGGTNYAQKFQG | 12 |
| | HCDR3 | VTYNIGWYIDY | 13 |
| | VH | EVQLVQSGAEVKKPGASVKVSCKASGYTFTGYYMHWVRQAPGQGLEWMG | 14 |
| | | RINPNSGGTNYAQKFQGRVTMTRDTSISTAYMELSGLRSDDTAVYFCAR | |
| | | VTYNIGWYIDYWGQGTLVTVSS | |
| | H chain | METDTLLLWVLLLWVPGSTGEVQLVQSGAEVKKPGASVKVSCKASGYTF | 15 |
| | (EP299) | TGYYMHWVRQAPGQGLEWMGRINPNSGGTNYAQKFQGRVTMTRDTSIST | 169 |
| | | AYMELSGLRSDDTAVYFCARVTYNIGWYIDYWGQGTLVTVSSASTKGPS | (no signal |
| | | VFPLAPSSKSTSGGTAALGCLVKDYFPEPVTVSWNSGALTSGVHTFPAV | peptide) |
| | | LQSSGLYSLSSVVTVPSSSLGTQTYICNVNHKPSNTKVDKKVEPKSCDK | |
| | | THTCPPCPAPELLGGPSVFLFPPKPKDTLMISRTPEVTCVVVDVSHEDP | |
| | | EVKFNWYVDGVEVHNAKTKPREEQYNSTYRVVSVLTVLHQDWLNGKEYK | |
| | | CKVSNKALPAPIEKTISKAKGQPREPQVYTLPPSRDELTKNQVSLTCLV | |
| | | KGFYPSDIAVEWESNGQPENNYKTTPPVLDSDGSFFLYSKLTVDKSRWQ | |
| | | QGNVFSCSVMHEALHNHYTQKSLSLSPGK | |
| | LCDR1 | GGNNIGSKGVH | 16 |
| | LCDR2 | YDTDRPS | 17 |
| | LCDR3 | QVWDSSSDHPV | 18 |
| | VL | QAVVTQPPSVSVAPGKTATITCGGNNIGSKGVHWYQQKPGQAPVLVIYY | 19 |
| | | DTDRPSGIPERLSGSNSGNTATLTISRVEAGDEADYYCQVWDSSSDHPV | |
| | | FGGGTKLTVL | |
| | L chain | METDTLLLWVLLLWVPGSTGQAVVTQPPSVSVAPGKTATITCGGNNIGS | 20 |
| | (EP289) | KGVHWYQQKPGQAPVLVIYYDTDRPSGIPERLSGSNSGNTATLTISRVE | 170 |
| | | AGDEADYYCQVWDSSSDHPVFGGGTKLTVLGQPKANPTVTLFPPSSEEL | (no signal |
| | | QANKATLVCLISDFYPGAVTVAWKADGSPVKAGVETTKPSKQSNNKYAA | peptide) |
| | | SSYLSLTPEQWKSHRSYSCQVTHEGSTVEKTVAPTECS | |
| EP034- | HCDR1 | GYYMH | 11 |
| E01 | HCDR2 | WINPNSGGTNYAQKFQG | 21 |
| | HCDR3 | GMWVPSITMIVGGGAFDI | 22 |
| | VH | EVQLVQSGAEVKKPGASVKVSCKASGYTFTGYYMHWVRQAPGQGLEWMG | 23 |
| | | WINPNSGGTNYAQKFQGRVTMTRDTSISTAYMELSRLRSDDTAVYYCAR | |
| | | GMWVPSITMIVGGGAFDIWGQGTMVTVSS | |
| | H chain | METDTLLLWVLLLWVPGSTGEVQLVQSGAEVKKPGASVKVSCKASGYTF | 24 |
| | (EP296) | TGYYMHWVRQAPGQGLEWMGWINPNSGGTNYAQKFQGRVTMTRDTSIST | 171 |
| | | AYMELSRLRSDDTAVYYCARGMWVPSITMIVGGGAFDIWGQGTMVTVSS | (no signal |
| | | ASTKGPSVFPLAPSSKSTSGGTAALGCLVKDYFPEPVTVSWNSGALTSG | peptide) |
| | | VHTFPAVLQSSGLYSLSSVVTVPSSSLGTQTYICNVNHKPSNTKVDKKV | |
| | | EPKSCDKTHTCPPCPAPELLGGPSVFLFPPKPKDTLMISRTPEVTCVVV | |
| | | DVSHEDPEVKFNWYVDGVEVHNAKTKPREEQYNSTYRVVSVLTVLHQDW | |
| | | LNGKEYKCKVSNKALPAPIEKTISKAKGQPREPQVYTLPPSRDELTKNQ | |
| | | VSLTCLVKGFYPSDIAVEWESNGQPENNYKTTPPVLDSDGSFFLYSKLT | |
| | | VDKSRWQQGNVFSCSVMHEALHNHYTQKSLSLSPGK | |
| | LCDR1 | RSSESLLHRNGFNYLD | 25 |
| | LCDR2 | MGSYRAS | 26 |
| | LCDR3 | MQALQSPPLYT | 27 |
| | VL | DVVMTQSPLSLPVTPGEPASMSCRSSESLLHRNGFNYLDWYVQRPGQSP | 28 |
| | | KLLIYMGSYRASGVPDRFSGRGSGTDFALKISRVEAEDVGVYYCMQALQ | |
| | | SPPLYTFGPGTKLEIK | |
| | L chain | METDTLLLWVLLLWVPGSTGDVVMTQSPLSLPVTPGEPASMSCRSSESL | 29 |
| | (EP286) | LHRNGFNYLDWYVQRPGQSPKLLIYMGSYRASGVPDRFSGRGSGTDFAL | 172 |
| | | KISRVEAEDVGVYYCMQALQSPPLYTFGPGTKLEIKRTVAAPSAVFIFP | (no signal |
| | | PSDEQLKSGTASVVCLLNNFYPREAKVQWKVDNALQSGNSQESVTEQDS | peptide) |
| | | KDSTYSLSSTLTLSKADYEKHKVYACEVTHQGLSSPVTKSFNRGEC | |
| EP47- | HCDR1 | GYYWS | 30 |
| F02 | HCDR2 | EINHSGSTNYNPSLKS | 31 |
| | HCDR3 | GWYLGFDY | 32 |
| | VH | QVQLQQWGAGLLKPSETLSLTCAVYGGSFSGYYWSWIRQPPGKGLEWIG | 33 |
| | | EINHSGSTNYNPSLKSRVTISVDTSKNQFSLKLSSVTAADTAVYYCARG | |
| | | WYLGFDYWGQGTLVTVSS | |
| | LCDR1 | TGTSRDVGGYDYVS | 34 |
| | LCDR2 | GVSERPS | 35 |
| | LCDR3 | CSYAGSFTWV | 36 |
| | VL | QSVLTQPRSVSGSPGQSVTISCTGTSRDVGGYDYVSWYQQYPGKAPKLM | 37 |
| | | ISGVSERPSGVPDRFTGSRSANTASLTISGLQTDDEANYYCCSYAGSFT | |
| | | WVFGDGTKLTVL | |
| EP030- | HCDR1 | SSSYYWG | 38 |
| F02 | HCDR2 | SIYYSGSTYYNPSLKS | 39 |
| | HCDR3 | QANQVVPAAIPWAKPGGTPPNWFDP | 40 |
| | VH | QVQLQESGPGLVKPSETLSLTCTVSGGSISSSSYYWGWIRQPPGKGLEW | 41 |
| | | IGSIYYSGSTYYNPSLKSRVTISVDTSKNQFSLKLSSVTAADTAVYYCA | |
| | | RQANQVVPAAIPWAKPGGTPPNWFDPWGQGTLVTVSS | |
| | LCDR1 | GGNNIGSKSVH | 42 |
| | LCDR2 | YDSARPS | 43 |
| | LCDR3 | QVWDSSSDHPRV | 44 |
| | VL | QLVLTQPPSVSVAPGETARITCGGNNIGSKSVHWYQQKPGQAPVLVIYY | 45 |
| | | DSARPSGIPERVSGSNSGNTATLTISRVEAGDEADYYCQVWDSSSDHPR | |
| | | VFGTGTKLTVL | |
| EP030- | HCDR1 | DYAVS | 46 |
| B10 | HCDR2 | FIESKPYGETREYAASVRG | 47 |
| | HCDR3 | VVAWVAY | 48 |
| | VH | QVQLVESGGDLVNPGRSLRLACTGAGFTFGDYAVSWFRQAPGKGLEWVG | 49 |
| | | FIESKPYGETREYAASVRGRFTISRDDSKGIAYLQMNGLKTEDTGVYYC | |
| | | SSVVAWVAYWGQGTLVTVSS | |
| | LCDR1 | RASQSVTTYLN | 50 |
| | LCDR2 | GTSALQS | 51 |
| | LCDR3 | QQSYSLPPT | 52 |
| | VL | AIQLTQSPSSLSASIGDRVTITCRASQSVTTYLNWYHQKPGKAPTFLIY | 53 |
| | | GTSALQSGVPSRFSGSGSGTDFTLTISSLQPEDFGIYYCQQSYSLPPTF | |
| | | GGGTNVQIRR | |
| EP030- | HCDR1 | SSAVQ | 54 |
| C11 | HCDR2 | WIVVGSGNTNYAQKFQE | 55 |
| | HCDR3 | DGDYDIEGALDY | 56 |
| | VH | QMQLVQSKPEVKKPGTSVKVSCKASGFTFTSSAVQWVRQARGQRLEWIG | 57 |
| | | WIVVGSGNTNYAQKFQERVTITRDMSTSTAYMELSSLRSEDTAVYYCAA | |
| | | DGDYDIEGALDYWGQGTLVTVSS | |
| | LCDR1 | RASQSVSSYLA | 58 |
| | LCDR2 | DASNRAT | 59 |
| | LCDR3 | QQRSNWIT | 60 |
| | VL | EIVLTQSPAILSLSPGERATLSCRASQSVSSYLAWYQQKPGQAPRLLIY | 61 |
| | | DASNRATGIPARFSGSGSGTDFTLTISSLEPEDFAVYYCQQRSNWITFG | |
| | | GGTKVEIKR | |
| EP023- | HCDR1 | SYYMH | 62 |
| E10 | HCDR2 | IINPSGGSTSYAQKFQG | 63 |
| | HCDR3 | SGTMTHLKGE | 64 |
| | VH | EVQLVESGAEVKKPGASVKVSCKASGYTFTSYYMHWVRQAPGQGLEWMG | 65 |
| | | IINPSGGSTSYAQKFQGRVTMTRDTSTSTVYMELSSLRSEDTAVYYCAS | |
| | | SGTMTHLKGEWGQGTLVTVSS | |
| | LCDR1 | RSSESLLHSSGYNFLD | 66 |
| | LCDR2 | LGSTRAS | 67 |
| | LCDR3 | MQALETPT | 68 |
| | VL | DIVMTQSPLSLPVTPGEPASISCRSSESLLHSSGYNFLDWYLQKPGQSP | 69 |
| | | QLLIYLGSTRASGVPDRFSGSGSGTDFTLKISRVDAEDVGVYYCMQALE | |
| | | TPTFGQGTKLEIKR | |
| EP023- | HCDR1 | SYAIS | 1 |
| D06 | HCDR2 | GIIPIFGTANYAQKFQG | 2 |
| | HCDR3 | SRPVRGAYY | 70 |
| | VH | QVQLVESGAEVKKPGSSVKVSCKASGGTFSSYAISWVRQAPGQGLEWMG | 71 |
| | | GIIPIFGTANYAQKFQGRVTITADESTSTAYMELSSLRSEDTAVYYCAR | |
| | | SRPVRGAYYWGQGTLVTVSS | |
| | LCDR1 | RASQIVNSNYLN | 72 |
| | LCDR2 | GVSNLQV | 73 |
| | LCDR3 | QQSYTTPRYS | 74 |
| | VL | DIQMTQSPSSLSASVGDRVTITCRASQIVNSNYLNWYQQKPGKAPNLLI | 75 |
| | | YGVSNLQVGVPSRFSGSGSGTDFTLSISSLQVEDSATYYCQQSYTTPRY | |
| | | SFGQGTKLEIRR | |
| EP023- | HCDR1 | SYGMH | 76 |
| H06 | HCDR2 | FIRYDGFNKYYADSVKG | 77 |
| | HCDR3 | VGRDGYNWFDY | 78 |
| | VH | QVQLVESGGGVVQPGGSLRLSCAASGFTFSSYGMHWVRQAPGEGLEWVA | 79 |
| | | FIRYDGFNKYYADSVKGRFTISRDNSKNTLYLQLNSLRAEDTAVYYCAK | |
| | | VGRDGYNWFDYWGQGTLVTVSS | |
| | LCDR1 | GGDNIAIKTVH | 80 |
| | LCDR2 | DDSDRPS | 81 |
| | LCDR3 | QVWDSRPDHPV | 82 |
| | VL | QPVLTQPPSMSVAPGQTARITCGGDNIAIKTVHWYQQRPGQAPVLVVHD | 83 |
| | | DSDRPSGIPERFSGSNSGNTAALTISRVEAGDEADYYCQVWDSRPDHPV | |
| | | FGGGTKVTVLR | |
| EP029- | HCDR1 | SNRAAWS | 84 |
| C04 | HCDR2 | RTYYRSKWYNDYAVSVES | 85 |
| | HCDR3 | GSFESTWL | 86 |
| | VH | QLQLQQSGPGLVKPSQTLSLTCAISGDSVSSNRAAWSWIRQSPSRGLEW | 87 |
| | | LGRTYYRSKWYNDYAVSVESRIIINPDTSKNQFSLQLNPVTPEDTAVYY | |
| | | CARGSFESTWLWGQGTLVTVSS | |
| | LCDR1 | TRSGGSIANNYVH | 88 |
| | LCDR2 | QDNQRRS | 89 |
| | LCDR3 | QSFDNNNVV | 90 |
| | VL | QAVVTQPHSVSESPGKTVTISCTRSGGSIANNYVHWYQQRPGSFPTALI | 91 |
| | | YQDNQRRSGVPDRFSGSIDSSSNSASLTISGLKTEDEAEYYCQSFDNNN | |
| | | VVFGGGTQLTVL | |
| EP032- | HCDR1 | SYAIS | 1 |
| D10 | HCDR2 | GIIPIFGTANYAQKFQG | 2 |
| | HCDR3 | DLAIVYGSGSYYNHHPRIDYYYYGMDV | 92 |
| | VH | EVQLVESGAEVKKPGSSVKVSCKASGGTFSSYAISWVRQAPGQGLEWMG | 93 |
| | | GIIPIFGTANYAQKFQGRVAITADESTSTAYMELSSLRSEDTAVYYCAR | |
| | | DLAIVYGSGSYYNHHPRIDYYYYGMDVWGQGTTVTVSS | |
| | LCDR1 | AGTSSDIGAYNYVS | 94 |
| | LCDR2 | DVSKRPS | 95 |
| | LCDR3 | FSYAGSYTLV | 96 |
| | VL | QSVLTQPRSVSGSPGQSVTISCAGTSSDIGAYNYVSWYQQHPGKAPTLM | 97 |
| | | INDVSKRPSGVPDRFSGSKSGNTASLTISGLQAEDEGDYYCFSYAGSYT | |
| | | LVFGGGTQLTVL | |
| EP032- | HCDR1 | GYYMH | 11 |
| E06 | HCDR2 | RINPNSGGTNYAQKFQG | 12 |
| | HCDR3 | ADYQWVGAIFRLNAFDI | 98 |
| | VH | QVQLVESGAEVKKPGASVKVSCKASGYTFTGYYMHWVRQAPGQGLEWMG | 99 |
| | | RINPNSGGTNYAQKFQGRFTISRDNSKTTLFLQMNSLRAEDTAVYYCAK | |
| | | ADYQWVGAIFRLNAFDIWGQGTMVTVSS | |
| | LCDR1 | RASQGISNYLA | 100 |
| | LCDR2 | AASTLQS | 101 |
| | LCDR3 | QKYNSAPYT | 102 |
| | VL | VIWMTQSPSSLSASVGDRVTITCRASQGISNYLAWYQQKPGKVPKLLIY | 103 |
| | | AASTLQSGVPSRFSGSGSGTDFTLTISSLQPEDVATYYCQKYNSAPYTF | |
| | | GQGTKLEIK | |

Sequence Table 2: Polypeptides for Bispecific Antibodies and Antibody-IL2 Protein
Complexes

| | | | SEQ ID |
|---|---|---|---|
| ID | Description | Sequences | NO |
| EP290 | WT_IL2_ | METDTLLLWVLLLWVPGSTGAPASSSTKKTQLQLEHLLLDLQMILNGINN | With |
| | IgG1_Knob | YKNPKLTRMLTFKFYMPKKATELKHLQCLEEELKPLEEVLNLAQSKNFHL | Signal |
| | | RPRDLISNINVIVLELKGSETTFMCEYADETATIVEFLNRWITFSQSIIS | Peptide: |
| | | TLTDKTHTCPPCPAPEAAGGPSVFLFPPKPKDTLMISRTPEVTCVVVDVS | 104 |
| | | HEDPEVKFNWYVDGVEVHNAKTKPREEQYNSTYRVVSVLTVLHQDWLNGK | Without |
| | | EYKCKVSNKALGAPIEKTISKAKGQPREPQVYTLPPCRDELTKNQVSLWC | Signal |
| | | LVKGFYPSDIAVEWESNGQPENNYKTTPPVLDSDGSFFLYSALTVDKSRW | Peptide: |
| | | QQGNVFSCSVMHEALHNHYTQKSLSLSPGK | 105 |
| EP297 | B3_ | METDTLLLWVLLLWVPGSTGAPASSSTKKTQLQLEHLLLDLQMILNGINN | With |
| | IgG1_Knob | YKNPKLTEMLTFKFYMPKKATELKHLQCLEEELKPLEEVLNLAQSKNFHL | Signal |
| | | TARDAVDNMRVIIQELKGSETTFMCEYADETATIVEFLNRWITFSQSIIS | Peptide: |
| | | TLTDKTHTCPPCPAPEAAGGPSVFLFPPKPKDTLMISRTPEVTCVVVDVS | 106 |
| | | HEDPEVKFNWYVDGVEVHNAKTKPREEQYNSTYRVVSVLTVLHQDWLNGK | Without |
| | | EYKCKVSNKALGAPIEKTISKAKGQPREPQVYTLPPCRDELTKNQVSLWC | Signal |
| | | LVKGFYPSDIAVEWESNGQPENNYKTTPPVLDSDGSFFLYSALTVDKSRW | Peptide: |
| | | QQGNVFSCSVMHEALHNHYTQKSLSLSPGK | 107 |
| EP369 | Anti- | METDTLLLWVLLLWVPGSTGEVQLVESGGGLVQPGGSLRLSCAASGFTFS | With |
| | CD3, | TYAMNWVRQAPGKGLEWVGRIRSKYNNYATYYADSVKGRFTISRDDSKNT | Signal |
| | sp34, Sc | LYLQMNSLRAEDTAVYYCVRHGNFGDSYVSWFAYWGQGTLVTVSSGGGGS | Peptide:: |
| | Fv-Fc, | GGGGSGGGGSGGGGSQAVVTQEPSLTVSPGGTVTLTCGSSTGAVTTSNYA | 108 |
| | Knob | NWVQQKPGKSPRGLIGGTNKRAPGVPARFSGSLLGGKAALTISGAQPEDE | Without |
| | | ADYYCALWYSNHWVFGGGTKLTVLEPKSSDKTHTCPPCPAPEAAGGPSVF | Signal |
| | | LFPPKPKDTLMISRTPEVTCVVVDVSHEDPEVKFNWYVDGVEVHNAKTKP | Peptide: |
| | | REEQYNSTYRVVSVLTVLHQDWLNGKEYKCKVSNKALGAPIEKTISKAKG | 109 |
| | | QPREPQVYTLPPCRDELTKNQVSLWCLVKGFYPSDIAVEWESNGQPENNY | |
| | | KTTPPVLDSDGSFFLYSALTVDKSRWQQGNVFSCSVMHEALHNHYTQKSL | |
| | | SLSPGK | |
| EP370 | EP34B09_ | METDTLLLWVLLLWVPGSTGEVQLVQSGAEVKKPGASVKVSCKASGYTFT | With |
| | HC_ | GYYMHWVRQAPGQGLEWMGRINPNSGGTNYAQKFQGRVTMTRDTSISTAY | Signal |
| | NOADCC_ | MELSGLRSDDTAVYFCARVTYNIGWYIDYWGQGTLVTVSSASTKGPSVFP | Peptide: |
| | Hole | LAPSSKSTSGGTAALGCLVKDYFPEPVTVSWNSGALTSGVHTFPAVLQSS | 110 |
| | | GLYSLSSVVTVPSSSLGTQTYICNVNHKPSNTKVDKKVEPKSCDKTHTCP | Without |
| | | PCPAPEAAGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LGAPIEKTISKAKGQPREPQVCTLPPSRDELTKNQVSLSCAVKGFYPSDI | 111 |
| | | AVEWESNGQPENNYKTTPPVLDSDGSFKLVSKLTVDKSRWQQGNVFSCSV | |
| | | MHEALHNHYTQKSLSLSPGK | |
| EP371 | EP34B09_ | METDTLLLWVLLLWVPGSTGEVQLVQSGAEVKKPGASVKVSCKASGYTFT | With |
| | WTIL2_ | GYYMHWVRQAPGQGLEWMGRINPNSGGTNYAQKFQGRVTMTRDTSISTAY | Signal |
| | HC_noADCC_ | MELSGLRSDDTAVYFCARVTYNIGWYIDYWGQGTLVTVSSASTKGPSVFP | Peptide: |
| | Hole | LAPSSKSTSGGTAALGCLVKDYFPEPVTVSWNSGALTSGVHTFPAVLQSS | 112 |
| | | GLYSLSSVVTVPSSSLGTQTYICNVNHKPSNTKVDKKVEPKSCDKTHTCP | Without |
| | | PCPAPEAAGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LGAPIEKTISKAKGQPREPQVCTLPPSRDELTKNQVSLSCAVKGFYPSDI | 113 |
| | | AVEWESNGQPENNYKTTPPVLDSDGSFKLVSKLTVDKSRWQQGNVFSCSV | |
| | | MHEALHNHYTQKSLSLSPGKGGGGSGGGGSGGGGSGGGGSAPASSSTKKT | |
| | | QLQLEHLLLDLQMILNGINNYKNPKLTRMLTFKFYMPKKATELKHLQCLE | |
| | | EELKPLEEVLNLAQSKNFHLRPRDLISNINVIVLELKGSETTFMCEYADE | |
| | | TATIVEFLNRWITFSQSIISTLT | |
| EP372 | EP34B09_ | METDTLLLWVLLLWVPGSTGEVQLVQSGAEVKKPGASVKVSCKASGYTFT | With |
| | B03IL2_ | GYYMHWVRQAPGQGLEWMGRINPNSGGTNYAQKFQGRVTMTRDTSISTAY | Signal |
| | HC_noADCC_ | MELSGLRSDDTAVYFCARVTYNIGWYIDYWGQGTLVTVSSASTKGPSVFP | Peptide: |
| | Hole | LAPSSKSTSGGTAALGCLVKDYFPEPVTVSWNSGALTSGVHTFPAVLQSS | 114 |
| | | GLYSLSSVVTVPSSSLGTQTYICNVNHKPSNTKVDKKVEPKSCDKTHTCP | Without |
| | | PCPAPEAAGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LGAPIEKTISKAKGQPREPQVCTLPPSRDELTKNQVSLSCAVKGFYPSDI | 115 |
| | | AVEWESNGQPENNYKTTPPVLDSDGSFKLVSKLTVDKSRWQQGNVFSCSV | |
| | | MHEALHNHYTQKSLSLSPGKGGGGSGGGGSGGGGSGGGGSAPASSSTKKT | |
| | | QLQLEHLLLDLQMILNGINNYKNPKLTRMLTFKFYMPKKATELKHLQCLE | |
| | | EELKPLEEVLNLAQSKNFHLTARDAVDNMRVIIQELKGSETTFMCEYADE | |
| | | TATIVEFLNRWITFSQSIISTLT | |
| EP373 | EP34B09_ | METDTLLLWVLLLWVPGSTGEVQLVQSGAEVKKPGASVKVSCKASGYTFT | With |
| | HC_ | GYYMHWVRQAPGQGLEWMGRINPNSGGTNYAQKFQGRVTMTRDTSISTAY | Signal |
| | wADCC_Knob | MELSGLRSDDTAVYFCARVTYNIGWYIDYWGQGTLVTVSSASTKGPSVFP | Peptide: |
| | | LAPSSKSTSGGTAALGCLVKDYFPEPVTVSWNSGALTSGVHTFPAVLQSS | 116 |
| | | GLYSLSSVVTVPSSSLGTQTYICNVNHKPSNTKVDKKVEPKSCDKTHTCP | Without |
| | | PCPAPELLGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LPAPIEKTISKAKGQPREPQVYTLPPCRDELTKNQVSLWCLVKGFYPSDI | 117 |
| | | AVEWESNGQPENNYKTTPPVLDSDGSFFLYSALTVDKSRWQQGNVFSCSV | |
| | | MHEALHNHYTQKSLSLSPGK | |
| EP374 | EP34B09_ | METDTLLLWVLLLWVPGSTGEVQLVQSGAEVKKPGASVKVSCKASGYTFT | With |
| | WTIL2HC_ | GYYMHWVRQAPGQGLEWMGRINPNSGGTNYAQKFQGRVTMTRDTSISTAY | Signal |
| | wADCC_ | MELSGLRSDDTAVYFCARVTYNIGWYIDYWGQGTLVTVSSASTKGPSVFP | Peptide: |
| | Hole | LAPSSKSTSGGTAALGCLVKDYFPEPVTVSWNSGALTSGVHTFPAVLQSS | 118 |
| | | GLYSLSSVVTVPSSSLGTQTYICNVNHKPSNTKVDKKVEPKSCDKTHTCP | Without |
| | | PCPAPELLGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LPAPIEKTISKAKGQPREPQVCTLPPSRDELTKNQVSLSCAVKGFYPSDI | 119 |
| | | AVEWESNGQPENNYKTTPPVLDSDGSFKLVSKLTVDKSRWQQGNVFSCSV | |
| | | MHEALHNHYTQKSLSLSPGKGGGGSGGGGSGGGGSGGGGSAPASSSTKKT | |
| | | QLQLEHLLLDLQMILNGINNYKNPKLTRMLTFKFYMPKKATELKHLQCLE | |
| | | EELKPLEEVLNLAQSKNFHLRPRDLISNINVIVLELKGSETTFMCEYADE | |
| | | TATIVEFLNRWITFSQSIISTLT | |
| EP375 | EP34B09_ | METDTLLLWVLLLWVPGSTGEVQLVQSGAEVKKPGASVKVSCKASGYTFT | With |
| | K35LR38D_ | GYYMHWVRQAPGQGLEWMGRINPNSGGTNYAQKFQGRVTMTRDTSISTAY | Signal |
| | F42R_ | MELSGLRSDDTAVYFCARVTYNIGWYIDYWGQGTLVTVSSASTKGPSVFP | Peptide: |
| | IL_ | LAPSSKSTSGGTAALGCLVKDYFPEPVTVSWNSGALTSGVHTFPAVLQSS | 120 |
| | HC_wADCC_ | GLYSLSSVVTVPSSSLGTQTYICNVNHKPSNTKVDKKVEPKSCDKTHTCP | Without |
| | Hole | PCPAPEAAGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LGAPIEKTISKAKGQPREPQVCTLPPSRDELTKNQVSLSCAVKGFYPSDI | 121 |
| | | AVEWESNGQPENNYKTTPPVLDSDGSFKLVSKLTVDKSRWQQGNVFSCSV | |
| | | MHEALHNHYTQKSLSLSPGKGGGGSGGGGSGGGGSGGGGSAPASSSTKKT | |
| | | QLQLEHLLLDLQMILNGINNYKNPLLTDMLTRKFYMPKKATELKHLQCLE | |
| | | EELKPLEEVLNLAQSKNFHLRPRDLISNINVIVLELKGSETTFMCEYADE | |
| | | TATIVEFLNRWITFSQSIISTLT | |
| EP376 | EP34B09_ | METDTLLLWVLLLWVPGSTGEVQLVQSGAEVKKPGASVKVSCKASGYTFT | With |
| | R38DY45S_ | GYYMHWVRQAPGQGLEWMGRINPNSGGTNYAQKFQGRVTMTRDTSISTAY | Signal |
| | IL2_ | MELSGLRSDDTAVYFCARVTYNIGWYIDYWGQGTLVTVSSASTKGPSVFP | Peptide: |
| | HC_ | LAPSSKSTSGGTAALGCLVKDYFPEPVTVSWNSGALTSGVHTFPAVLQSS | 122 |
| | wADCC_Hole | GLYSLSSVVTVPSSSLGTQTYICNVNHKPSNTKVDKKVEPKSCDKTHTCP | Without |
| | | PCPAPEAAGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LGAPIEKTISKAKGQPREPQVCTLPPSRDELTKNQVSLSCAVKGFYPSDI | 123 |
| | | AVEWESNGQPENNYKTTPPVLDSDGSFKLVSKLTVDKSRWQQGNVFSCSV | |
| | | MHEALHNHYTQKSLSLSPGKGGGGSGGGGSGGGGSGGGGSAPASSSTKKT | |
| | | QLQLEHLLLDLQMILNGINNYKNPKLTDMLTFKFSMPKKATELKHLQCLE | |
| | | EELKPLEEVLNLAQSKNFHLRPRDLISNINVIVLELKGSETTFMCEYADE | |
| | | TATIVEFLNRWITFSQSIISTLT | |
| EP377 | EP34B09_ | METDTLLLWVLLLWVPGSTGEVQLVQSGAEVKKPGASVKVSCKASGYTFT | With |
| | B03IL2_ | GYYMHWVRQAPGQGLEWMGRINPNSGGTNYAQKFQGRVTMTRDTSISTAY | Signal |
| | HC_wADCC_ | MELSGLRSDDTAVYFCARVTYNIGWYIDYWGQGTLVTVSSASTKGPSVFP | Peptide: |
| | Hole | LAPSSKSTSGGTAALGCLVKDYFPEPVTVSWNSGALTSGVHTFPAVLQSS | 124 |
| | | GLYSLSSVVTVPSSSLGTQTYICNVNHKPSNTKVDKKVEPKSCDKTHTCP | Without |
| | | PCPAPELLGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LPAPIEKTISKAKGQPREPQVCTLPPSRDELTKNQVSLSCAVKGFYPSDI | 125 |
| | | AVEWESNGQPENNYKTTPPVLDSDGSFKLVSKLTVDKSRWQQGNVFSCSV | |
| | | MHEALHNHYTQKSLSLSPGKGGGGSGGGGSGGGGSGGGGSAPASSSTKKT | |
| | | QLQLEHLLLDLQMILNGINNYKNPKLTRMLTFKFYMPKKATELKHLQCLE | |
| | | EELKPLEEVLNLAQSKNFHLTARDAVDNMRVIIQELKGSETTFMCEYADE | |
| | | TATIVEFLNRWITFSQSIISTLT | |
| EP378 | EP34B09_ | METDTLLLWVLLLWVPGSTGEVQLVQSGAEVKKPGASVKVSCKASGYTFT | With |
| | HC_NoADCC_ | GYYMHWVRQAPGQGLEWMGRINPNSGGTNYAQKFQGRVTMTRDTSISTAY | Signal |
| | Knob | MELSGLRSDDTAVYFCARVTYNIGWYIDYWGQGTLVTVSSASTKGPSVFP | Peptide: |
| | | LAPSSKSTSGGTAALGCLVKDYFPEPVTVSWNSGALTSGVHTFPAVLQSS | 126 |
| | | GLYSLSSVVTVPSSSLGTQTYICNVNHKPSNTKVDKKVEPKSCDKTHTCP | Without |
| | | PCPAPEAAGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LGAPIEKTISKAKGQPREPQVYTLPPCRDELTKNQVSLWCLVKGFYPSDI | 127 |
| | | AVEWESNGQPENNYKTTPPVLDSDGSFFLYSALTVDKSRWQQGNVFSCSV | |
| | | MHEALHNHYTQKSLSLSPGK | |
| EP379 | EP34B09_ | METDTLLLWVLLLWVPGSTGEVQLVQSGAEVKKPGASVKVSCKASGYTFT | With |
| | K35L_R38D_ | GYYMHWVRQAPGQGLEWMGRINPNSGGTNYAQKFQGRVTMTRDTSISTAY | Signal |
| | F42R_ | MELSGLRSDDTAVYFCARVTYNIGWYIDYWGQGTLVTVSSASTKGPSVFP | Peptide: |
| | WTIL2_ | LAPSSKSTSGGTAALGCLVKDYFPEPVTVSWNSGALTSGVHTFPAVLQSS | 128 |
| | HC_NoADCC_ | GLYSLSSVVTVPSSSLGTQTYICNVNHKPSNTKVDKKVEPKSCDKTHTCP | Without |
| | Hole | PCPAPEAAGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LGAPIEKTISKAKGQPREPQVCTLPPSRDELTKNQVSLSCAVKGFYPSDI | 129 |
| | | AVEWESNGQPENNYKTTPPVLDSDGSFKLVSKLTVDKSRWQQGNVFSCSV | |
| | | MHEALHNHYTQKSLSLSPGKGGGGSGGGGSGGGGSGGGGSAPASSSTKKT | |
| | | QLQLEHLLLDLQMILNGINNYKNPLLTDMLTRKFYMPKKATELKHLQCLE | |
| | | EELKPLEEVLNLAQSKNFHLRPRDLISNINVIVLELKGSETTFMCEYADE | |
| | | TATIVEFLNRWITFSQSIISTLT | |
| EP380 | EP34B09_ | METDTLLLWVLLLWVPGSTGEVQLVQSGAEVKKPGASVKVSCKASGYTFT | With |
| | R38D_Y45S_ | GYYMHWVRQAPGQGLEWMGRINPNSGGTNYAQKFQGRVTMTRDTSISTAY | Signal |
| | IL2_ | MELSGLRSDDTAVYFCARVTYNIGWYIDYWGQGTLVTVSSASTKGPSVFP | Peptide: |
| | HC_NoADCC_ | LAPSSKSTSGGTAALGCLVKDYFPEPVTVSWNSGALTSGVHTFPAVLQSS | 130 |
| | Hole | GLYSLSSVVTVPSSSLGTQTYICNVNHKPSNTKVDKKVEPKSCDKTHTCP | Without |
| | | PCPAPEAAGGPSVFLFPPKPKDTLMISRTPEVTCVVVDVSHEDPEVKFNW | Signal |
| | | YVDGVEVHNAKTKPREEQYNSTYRVVSVLTVLHQDWLNGKEYKCKVSNKA | Peptide: |
| | | LGAPIEKTISKAKGQPREPQVCTLPPSRDELTKNQVSLSCAVKGFYPSDI | 131 |
| | | AVEWESNGQPENNYKTTPPVLDSDGSFKLVSKLTVDKSRWQQGNVFSCSV | |
| | | MHEALHNHYTQKSLSLSPGKGGGGSGGGGSGGGGSGGGGSAPASSSTKKT | |
| | | QLQLEHLLLDLQMILNGINNYKNPKLTDMLTFKFSMPKKATELKHLQCLE | |
| | | EELKPLEEVLNLAQSKNFHLRPRDLISNINVIVLELKGSETTFMCEYADE | |
| | | TATIVEFLNRWITFSQSIISTLT | |
| EP437 | Anti- | METDTLLLWVLLLWVPGSTGDIKLQQSGAELARPGASVKMSCKTSGYTFT | With |
| | CD3, OKT3_ | RYTMHWVKQRPGQGLEWIGYINPSRGYTNYNQKFKDKATLTTDKSSSTAY | Signal |
| | ScFv_ | MQLSSLTSEDSAVYYCARYYDDHYCLDYWGQGTTLTVSSGGGGSGGGGSG | Peptide: |
| | Knob | GGGSGGGGSDIQLTQSPAIMSASPGEKVTMTCRASSSVSYMNWYQQKSGT | 132 |
| | | SPKRWIYDTSKVASGVPYRFSGSGSGTSYSLTISSMEAEDAATYYCQQWS | Without |
| | | SNPLTFGAGTKLELKEPKSSDKTHTCPPCPAPEAAGGPSVFLFPPKPKDT | Signal |
| | | LMISRTPEVTCVVVDVSHEDPEVKFNWYVDGVEVHNAKTKPREEQYNSTY | Peptide: |
| | | RVVSVLTVLHQDWLNGKEYKCKVSNKALGAPIEKTISKAKGQPREPQVYT | 133 |
| | | LPPCRDELTKNQVSLWCLVKGFYPSDIAVEWESNGQPENNYKTTPPVLDS | |
| | | DGSFFLYSALTVDKSRWQQGNVFSCSVMHEALHNHYTQKSLSLSPGK | |
| EP452 | 4-1BB, | METDTLLLWVLLLWVPGSTGDPAGLLDLRQGMFAQLVAQNVLLIDGPLSW | With |
| | monomer | YSDPGLAGVSLTGGLSYKEDTKELVVAKAGVYYVFFQLELRRVVAGEGSG | Signal |
| | fused | SVSLALHLQPLRSAAGAAALALTVDLPPASSEARNSAFGFQGRLLHLSAG | Peptide: |
| | to OKT3 | QRLGVHLHTEARARHAWQLTQGATVLGLFRVTPEIPAGGGGSGGGGSGGG | 134 |
| | ScFv- | GSGGGGSDIKLQQSGAELARPGASVKMSCKTSGYTFTRYTMHWVKQRPGQ | Without |
| | Fc, Knob | GLEWIGYINPSRGYTNYNQKFKDKATLTTDKSSSTAYMQLSSLTSEDSAV | Signal |
| | | YYCARYYDDHYCLDYWGQGTTLTVSSGGGGSGGGGSGGGGSGGGGSDIQL | Peptide: |
| | | TQSPAIMSASPGEKVTMTCRASSSVSYMNWYQQKSGTSPKRWIYDTSKVA | 135 |
| | | SGVPYRFSGSGSGTSYSLTISSMEAEDAATYYCQQWSSNPLTFGAGTKLE | |
| | | LKEPKSSDKTHTCPPCPAPEAAGGPSVFLFPPKPKDTLMISRTPEVTCVV | |
| | | VDVSHEDPEVKFNWYVDGVEVHNAKTKPREEQYNSTYRVVSVLTVLHQDW | |
| | | LNGKEYKCKVSNKALGAPIEKTISKAKGQPREPQVYTLPPCRDELTKNQV | |
| | | SLWCLVKGFYPSDIAVEWESNGQPENNYKTTPPVLDSDGSFFLYSALTVD | |
| | | KSRWQQGNVFSCSVMHEALHNHYTQKSLSLSPGK | |
| EP453 | Cd86, | METDTLLLWVLLLWVPGSTGLKIQAYFNETADLPCQFANSQNQSLSELVV | With |
| | 26-134, | FWQDQENLVLNEVYLGKEKFDSVHSKYMGRTSFDSDSWTLRLHNLQIKDK | Signal |
| | fused | GLYQCIIHHKKPTGMIRIHQMNSELSVLAGGGGSGGGGSGGGGSGGGGSD | Peptide: |
| | to OKT3 | IKLQQSGAELARPGASVKMSCKTSGYTFTRYTMHWVKQRPGQGLEWIGYI | 136 |
| | ScFv-Fc | NPSRGYTNYNQKFKDKATLTTDKSSSTAYMQLSSLTSEDSAVYYCARYYD | Without |
| | Knob | DHYCLDYWGQGTTLTVSSGGGGSGGGGSGGGGSGGGGSDIQLTQSPAIMS | Signal |
| | | ASPGEKVTMTCRASSSVSYMNWYQQKSGTSPKRWIYDTSKVASGVPYRFS | Peptide: |
| | | GSGSGTSYSLTISSMEAEDAATYYCQQWSSNPLTFGAGTKLELKEPKSSD | 137 |
| | | KTHTCPPCPAPEAAGGPSVFLFPPKPKDTLMISRTPEVTCVVVDVSHEDP | |
| | | EVKFNWYVDGVEVHNAKTKPREEQYNSTYRVVSVLTVLHQDWLNGKEYKC | |
| | | KVSNKALGAPIEKTISKAKGQPREPQVYTLPPCRDELTKNQVSLWCLVKG | |
| | | FYPSDIAVEWESNGQPENNYKTTPPVLDSDGSFFLYSALTVDKSRWQQGN | |
| | | VFSCSVMHEALHNHYTQKSLSLSPGK | |
| EP454 | Cd80, | METDTLLLWVLLLWVPGSTGVIHVTKEVKEVATLSCGHNVSVEELAQTRI | With |
| | 35-140, | YWQKEKKMVLTMMSGDMNIWPEYKNRTIFDITNNLSIVILALRPSDEGTY | Signal |
| | fused | ECVVLKYEKDAFKREHLAEVTLSVKAGGGGSGGGGSGGGGSGGGGSDIKL | Peptide: |
| | to OKT3 | QQSGAELARPGASVKMSCKTSGYTFTRYTMHWVKQRPGQGLEWIGYINPS | 138 |
| | ScFv- | RGYTNYNQKFKDKATLTTDKSSSTAYMQLSSLTSEDSAVYYCARYYDDHY | Without |
| | Fc, Knob | CLDYWGQGTTLTVSSGGGGSGGGGSGGGGSGGGGSDIQLTQSPAIMSASP | Signal |
| | | GEKVTMTCRASSSVSYMNWYQQKSGTSPKRWIYDTSKVASGVPYRFSGSG | Peptide: |
| | | SGTSYSLTISSMEAEDAATYYCQQWSSNPLTFGAGTKLELKEPKSSDKTH | 139 |
| | | TCPPCPAPEAAGGPSVFLFPPKPKDTLMISRTPEVTCVVVDVSHEDPEVK | |
| | | FNWYVDGVEVHNAKTKPREEQYNSTYRVVSVLTVLHQDWLNGKEYKCKVS | |
| | | NKALGAPIEKTISKAKGQPREPQVYTLPPCRDELTKNQVSLWCLVKGFYP | |
| | | SDIAVEWESNGQPENNYKTTPPVLDSDGSFFLYSALTVDKSRWQQGNVFS | |
| | | CSVMHEALHNHYTQKSLSLSPGK | |
| EP455 | IcosL, | METDTLLLWVLLLWVPGSTGDTQEKEVRAMVGSDVELSCACPEGSREDLN | With |
| | 18-135, | DVYVYWQTSESKTVVTYHIPQNSSLENVDSRYRNRALMSPAGMLRGDFSL | Signal |
| | fused | RLFNVTPQDEQKFHCLVLSQSLGFQEVLSVEVTLHVAGGGGSGGGGSGGG | Peptide: |
| | to OKT3 | GSGGGGSDIKLQQSGAELARPGASVKMSCKTSGYTFTRYTMHWVKQRPGQ | 140 |
| | ScFv- | GLEWIGYINPSRGYTNYNQKFKDKATLTTDKSSSTAYMQLSSLTSEDSAV | Without |
| | Fc, Knob | YYCARYYDDHYCLDYWGQGTTLTVSSGGGGSGGGGSGGGGSGGGGSDIQL | Signal |
| | | TQSPAIMSASPGEKVTMTCRASSSVSYMNWYQQKSGTSPKRWIYDTSKVA | Peptide: |
| | | SGVPYRFSGSGSGTSYSLTISSMEAEDAATYYCQQWSSNPLTFGAGTKLE | 141 |
| | | LKEPKSSDKTHTCPPCPAPEAAGGPSVFLFPPKPKDTLMISRTPEVTCVV | |
| | | VDVSHEDPEVKFNWYVDGVEVHNAKTKPREEQYNSTYRVVSVLTVLHQDW | |
| | | LNGKEYKCKVSNKALGAPIEKTISKAKGQPREPQVYTLPPCRDELTKNQV | |
| | | SLWCLVKGFYPSDIAVEWESNGQPENNYKTTPPVLDSDGSFFLYSALTVD | |
| | | KSRWQQGNVFSCSVMHEALHNHYTQKSLSLSPGK | |
| EP456 | Humanized | METDTLLLWVLLLWVPGSTGQVQLVQSGGGVVQPGRSLRLSCKASGYTFT | With |
| | OKT3, h12F6, | SYTMHWVRQAPGKGLEWIGYINPSSGYTKYNQKFKDRFTISADKSKSTAF | Signal |
| | ScFv_ | LQMDSLRPEDTGVYFCARWQDYDVYFDYWGQGTPVTVSSGGGGSGGGSGG | Peptide: |
| | Fc-Knob | GSGGGGSDIQMTQSPSSLSASVGDRVTMTCRASSSVSYMHWYQQTPGKAP | 142 |
| | | KPWIYATSNLASGVPSRFSGSGSGTDYTLTISSLQPEDIATYYCQQWSSN | Without |
| | | PPTFGQGTKLQITREPKSSDKTHTCPPCPAPEAAGGPSVFLFPPKPKDTL | Signal |
| | | MISRTPEVTCVVVDVSHEDPEVKFNWYVDGVEVHNAKTKPREEQYNSTYR | Peptide: |
| | | VVSVLTVLHQDWLNGKEYKCKVSNKALGAPIEKTISKAKGQPREPQVYTL | 143 |
| | | PPCRDELTKNQVSLWCLVKGFYPSDIAVEWESNGQPENNYKTTPPVLDSD | |
| | | GSFFLYSALTVDKSRWQQGNVFSCSVMHEALHNHYTQKSLSLSPGK | |
| EP457 | anti- | METDTLLLWVLLLWVPGSTGEVQLVQSGAEVKKPGASVKVSCKASGYTFT | With |
| | Nectin4- | GYYMHWVRQAPGQGLEWMGRINPNSGGTNYAQKFQGRVTMTRDTSISTAY | Signal |
| | B09- | MELSGLRSDDTAVYFCARVTYNIGWYIDYWGQGTLVTVSSASTKGPSVFP | Peptide: |
| | (g4S)2- | LAPSSKSTSGGTAALGCLVKDYFPEPVTVSWNSGALTSGVHTFPAVLQSS | 144 |
| | h12F6- | GLYSLSSVVTVPSSSLGTQTYICNVNHKPSNTKVDKKVEPKSCDGGGGSG | Without |
| | ScFv- | GGGSQVQLVQSGGGVVQPGRSLRLSCKASGYTFTSYTMHWVRQAPGKGLE | Signal |
| | IgG1, hole | WIGYINPSSGYTKYNQKFKDRFTISADKSKSTAFLQMDSLRPEDIGVYFC | Peptide: |
| | | ARWQDYDVYFDYWGQGTPVTVSSGGGGSGGGSGGGSGGGGSDIQMTQSPS | 145 |
| | | SLSASVGDRVTMTCRASSSVSYMHWYQQTPGKAPKPWIYATSNLASGVPS | |
| | | RFSGSGSGTDYTLTISSLQPEDIATYYCQQWSSNPPTFGQGTKLQITREP | |
| | | KSCDKTHTCPPCPAPEAAGGPSVFLFPPKPKDTLMISRTPEVTCVVVDVS | |
| | | HEDPEVKFNWYVDGVEVHNAKTKPREEQYNSTYRVVSVLTVLHQDWLNGK | |
| | | EYKCKVSNKALGAPIEKTISKAKGQPREPQVCTLPPSRDELTKNQVSLSC | |
| | | AVKGFYPSDIAVEWESNGQPENNYKTTPPVLDSDGSFKLVSKLTVDKSRW | |
| | | QQGNVFSCSVMHEALHNHYTQKSLSLSPGK | |
| EP458 | anti- | METDTLLLWVLLLWVPGSTGEVQLVQSGAEVKKPGASVKVSCKASGYTFT | With |
| | Nectin4- | GYYMHWVRQAPGQGLEWMGRINPNSGGTNYAQKFQGRVTMTRDTSISTAY | Signal |
| | B09- | MELSGLRSDDTAVYFCARVTYNIGWYIDYWGQGTLVTVSSASTKGPSVEP | Peptide: |
| | (g4S)2- | LAPSSKSTSGGTAALGCLVKDYFPEPVTVSWNSGALTSGVHTFPAVLQSS | 146 |
| | sp34- | GLYSLSSVVTVPSSSLGTQTYICNVNHKPSNTKVDKKVEPKSCDGGGGSG | Without |
| | ScFv- | GGGSEVQLVESGGGLVQPGGSLRLSCAASGFTFSTYAMNWVRQAPGKGLE | Signal |
| | IgG1, hole | WVGRIRSKYNNYATYYADSVKGRFTISRDDSKNTLYLQMNSLRAEDTAVY | Peptide: |
| | | YCVRHGNFGDSYVSWFAYWGQGTLVTVSSGGGGSGGGGSGGGGSGGGGSQ | 147 |
| | | AVVTQEPSLTVSPGGTVTLTCGSSTGAVTTSNYANWVQQKPGKSPRGLIG | |
| | | GTNKRAPGVPARFSGSLLGGKAALTISGAQPEDEADYYCALWYSNHWVFG | |
| | | GGTKLTVLEPKSCDKTHTCPPCPAPEAAGGPSVFLFPPKPKDTLMISRTP | |
| | | EVTCVVVDVSHEDPEVKFNWYVDGVEVHNAKTKPREEQYNSTYRVVSVLT | |
| | | VLHQDWLNGKEYKCKVSNKALGAPIEKTISKAKGQPREPQVCTLPPSRDE | |
| | | LTKNQVSLSCAVKGFYPSDIAVEWESNGQPENNYKTTPPVLDSDGSFKLV | |
| | | SKLTVDKSRWQQGNVFSCSVMHEALHNHYTQKSLSLSPGK | |
| EP499 | humanized | METDTLLLWVLLLWVPGSTGEVQLVQSGAEVKKPGASVKVSCKASGYTFT | With |
| | SP34, | GYYMHWVRQAPGQGLEWMGRINPNSGGTNYAQKFQGRVTMTRDTSISTAY | Signal |
| | QIV65546_ | MELSGLRSDDTAVYFCARVTYNIGWYIDYWGQGTLVTVSSASTKGPSVFP | Peptide: |
| | HC, | LAPSSKSTSGGTAALGCLVKDYFPEPVTVSWNSGALTSGVHTFPAVLQSS | 148 |
| | 3T2N_L_ | GLYSLSSVVTVPSSSLGTQTYICNVNHKPSNTKVDKKVEPKSCDGGGGSG | Without |
| | LC | GGGSEVQLVESGGGLVQPGGSLRLSCAASGFTFNTYAMNWVRQAPGKGLE | Signal |
| | | WVARIRSKYNNYATYYAASVKGRFTISRDDSKNSLFLQMNSLKTEDTAVY | Peptide: |
| | | YCVRHGNFGNSYVSWFAYWGQGTLVTVSSGGGGSGGGGSGGGGSGGGGSQ | 149 |
| | | TVVTQEPSLTVSPGGTVTLTCRSSTGAVTTSNYANWVQEKPDHLFTGLIG | |
| | | GTNKRAPGVPARFSGSLLGGKAALTLSGVQPEDEAIYFCALWYSNLWVFG | |
| | | SGTKVTVLEPKSCDKTHTCPPCPAPEAAGGPSVFLFPPKPKDTLMISRTP | |
| | | EVTCVVVDVSHEDPEVKFNWYVDGVEVHNAKTKPREEQYNSTYRVVSVLT | |
| | | VLHQDWLNGKEYKCKVSNKALGAPIEKTISKAKGQPREPQVCTLPPSRDE | |
| | | LTKNQVSLSCAVKGFYPSDIAVEWESNGQPENNYKTTPPVLDSDGSFKLV | |
| | | SKLTVDKSRWQQGNVFSCSVMHEALHNHYTQKSLSLSPGK | |
| EP500 | humanized | METDTLLLWVLLLWVPGSTGEVQLVQSGAEVKKPGASVKVSCKASGYTFT | With |
| | SP34, | GYYMHWVRQAPGQGLEWMGRINPNSGGTNYAQKFQGRVTMTRDTSISTAY | Signal |
| | QIV65546_ | MELSGLRSDDTAVYFCARVTYNIGWYIDYWGQGTLVTVSSASTKGPSVFP | Peptide: |
| | HC, | LAPSSKSTSGGTAALGCLVKDYFPEPVTVSWNSGALTSGVHTFPAVLQSS | 150 |
| | QFR45443_ | GLYSLSSVVTVPSSSLGTQTYICNVNHKPSNTKVDKKVEPKSCDGGGGSG | Without |
| | LC | GGGSEVQLVESGGGLVQPGGSLRLSCAASGFTFNTYAMNWVRQAPGKGLE | Signal |
| | | WVARIRSKYNNYATYYAASVKGRFTISRDDSKNSLFLQMNSLKTEDTAVY | Peptide: |
| | | YCVRHGNFGNSYVSWFAYWGQGTLVTVSSGGGGSGGGGSGGGGSGGGGSQ | 151 |
| | | AVVTQEPSLTVSPGGTVTLTCASSTGAVTTSNYANWVQEKPDHLFTGLIG | |
| | | GTNKRAPGVPARFSGSLLGGKAALTLSGVQPEDEAEYYCALWYSNLWVFG | |
| | | GGTKLTVLEPKSCDKTHTCPPCPAPEAAGGPSVFLFPPKPKDTLMISRTP | |
| | | EVTCVVVDVSHEDPEVKFNWYVDGVEVHNAKTKPREEQYNSTYRVVSVLT | |
| | | VLHQDWLNGKEYKCKVSNKALGAPIEKTISKAKGQPREPQVCTLPPSRDE | |
| | | LTKNQVSLSCAVKGFYPSDIAVEWESNGQPENNYKTTPPVLDSDGSFKLV | |
| | | SKLTVDKSRWQQGNVFSCSVMHEALHNHYTQKSLSLSPGK | |
| EP501 | humanized | METDTLLLWVLLLWVPGSTGEVQLVQSGAEVKKPGASVKVSCKASGYTFT | With |
| | SP34, | GYYMHWVRQAPGQGLEWMGRINPNSGGTNYAQKFQGRVTMTRDTSISTAY | Signal |
| | ABM67282_ | MELSGLRSDDTAVYFCARVTYNIGWYIDYWGQGTLVTVSSASTKGPSVFP | Peptide: |
| | HC, | LAPSSKSTSGGTAALGCLVKDYFPEPVTVSWNSGALTSGVHTFPAVLQSS | 152 |
| | 3T2N_L_ | GLYSLSSVVTVPSSSLGTQTYICNVNHKPSNTKVDKKVEPKSCDGGGGSG | Without |
| | LC | GGGSEVQLVESGGGLVQPRGSLRLSCAASGFTFNTYAMNWVRQAPGKGLE | Signal |
| | | WVARIRSKYNNYATYYAASVKGRFSISRDDSENALYLQMNSLKTEDTAVY | Peptide: |
| | | YCVRHGNFGNSYVSWFAYWGQGTLVTVSSGGGGSGGGGSGGGGSGGGGSQ | 153 |
| | | TVVTQEPSLTVSPGGTVTLTCRSSTGAVTTSNYANWVQEKPDHLFTGLIG | |
| | | GTNKRAPGVPARFSGSLLGGKAALTLSGVQPEDEAIYFCALWYSNLWVFG | |
| | | SGTKVTVLEPKSCDKTHTCPPCPAPEAAGGPSVFLFPPKPKDTLMISRTP | |
| | | EVTCVVVDVSHEDPEVKFNWYVDGVEVHNAKTKPREEQYNSTYRVVSVLT | |
| | | VLHQDWLNGKEYKCKVSNKALGAPIEKTISKAKGQPREPQVCTLPPSRDE | |
| | | LTKNQVSLSCAVKGFYPSDIAVEWESNGQPENNYKTTPPVLDSDGSFKLV | |
| | | SKLTVDKSRWQQGNVFSCSVMHEALHNHYTQKSLSLSPGK | |
| EP502 | humanized | METDTLLLWVLLLWVPGSTGEVQLVQSGAEVKKPGASVKVSCKASGYTFT | With |
| | SP34, | GYYMHWVRQAPGQGLEWMGRINPNSGGTNYAQKFQGRVTMTRDTSISTAY | Signal |
| | ABM67282_ | MELSGLRSDDTAVYFCARVTYNIGWYIDYWGQGTLVTVSSASTKGPSVFP | Peptide: |
| | HC, | LAPSSKSTSGGTAALGCLVKDYFPEPVTVSWNSGALTSGVHTFPAVLQSS | 154 |
| | QFR45443_ | GLYSLSSVVTVPSSSLGTQTYICNVNHKPSNTKVDKKVEPKSCDGGGGSG | Without |
| | LC | GGGSEVQLVESGGGLVQPRGSLRLSCAASGFTFNTYAMNWVRQAPGKGLE | Signal |
| | | WVARIRSKYNNYATYYAASVKGRFSISRDDSENALYLQMNSLKTEDTAVY | Peptide: |
| | | YCVRHGNFGNSYVSWFAYWGQGTLVTVSSGGGGSGGGGSGGGGSGGGGSQ | 155 |
| | | AVVTQEPSLTVSPGGTVTLTCASSTGAVTTSNYANWVQEKPDHLFTGLIG | |
| | | GTNKRAPGVPARFSGSLLGGKAALTLSGVQPEDEAEYYCALWYSNLWVFG | |
| | | GGTKLTVLEPKSCDKTHTCPPCPAPEAAGGPSVFLFPPKPKDTLMISRTP | |
| | | EVTCVVVDVSHEDPEVKFNWYVDGVEVHNAKTKPREEQYNSTYRVVSVLT | |
| | | VLHQDWLNGKEYKCKVSNKALGAPIEKTISKAKGQPREPQVCTLPPSRDE | |
| | | LTKNQVSLSCAVKGFYPSDIAVEWESNGQPENNYKTTPPVLDSDGSFKLV | |
| | | SKLTVDKSRWQQGNVFSCSVMHEALHNHYTQKSLSLSPGK | |
| EP544 | 2020EP47- | METDTLLLWVLLLWVPGSTGQVQLQQWGAGLLKPSETLSLTCAVYGGSFS | With |
| | F02_HC | GYYWSWIRQPPGKGLEWIGEINHSGSTNYNPSLKSRVTISVDTSKNQFSL | Signal |
| | | KLSSVTAADTAVYYCARGWYLGFDYWGQGTLVTVSSASTKGPSVFPLAPS | Peptide: |
| | | SKSTSGGTAALGCLVKDYFPEPVTVSWNSGALTSGVHTFPAVLQSSGLYS | 156 |
| | | LSSVVTVPSSSLGTQTYICNVNHKPSNTKVDKKVEPKSCDKTHTCPPCPA | Without |
| | | PELLGGPSVFLFPPKPKDTLMISRTPEVTCVVVDVSHEDPEVKFNWYVDG | Signal |
| | | VEVHNAKTKPREEQYNSTYRVVSVLTVLHQDWLNGKEYKCKVSNKALPAP | Peptide: |
| | | IEKTISKAKGQPREPQVYTLPPSRDELTKNQVSLTCLVKGFYPSDIAVEW | 157 |
| | | ESNGQPENNYKTTPPVLDSDGSFFLYSKLTVDKSRWQQGNVFSCSVMHEA | |
| | | LHNHYTQKSLSLSPGK | |
| EP545 | 2020EP47- | METDTLLLWVLLLWVPGSTGQSVLTQPRSVSGSPGQSVTISCTGTSRDVG | With |
| | F02_LC | GYDYVSWYQQYPGKAPKLMISGVSERPSGVPDRFTGSRSANTASLTISGL | Signal |
| | | QTDDEANYYCCSYAGSFTWVFGDGTKLTVLGQPKANPTVTLFPPSSEELQ | Peptide: |
| | | ANKATLVCLISDFYPGAVTVAWKADGSPVKAGVETTKPSKQSNNKYAASS | 158 |
| | | YLSLTPEQWKSHRSYSCQVTHEGSTVEKTVAPTECS | Without |
| | | | Signal |
| | | | Peptide: |
| | | | 159 |
| EP559 | EP034B09_ | METDTLLLWVLLLWVPGSTGEVQLVQSGAEVKKPGASVKVSCKASGYTFT | With |
| | IgG4 | GYYMHWVRQAPGQGLEWMGRINPNSGGTNYAQKFQGRVTMTRDTSISTAY | Signal |
| | HC, | MELSGLRSDDTAVYFCARVTYNIGWYIDYWGQGTLVTVSSASTKGPSVFP | Peptide: |
| | anti- | LAPCSRSTSESTAALGCLVKDYFPEPVTVSWNSGALTSGVHTFPAVLQSS | 160 |
| | Nectin4 | GLYSLSSVVTVPSSSLGTKTYTCNVDHKPSNTKVDKRVESKYGPPCPPCP | Without |
| | | APEFLGGPSVFLFPPKPKDTLMISRTPEVTCVVVDVSQEDPEVQFNWYVD | Signal |
| | | GVEVHNAKTKPREEQFNSTYRVVSVLTVLHQDWLNGKEYKCKVSNKGLPS | Peptide: |
| | | SIEKTISKAKGQPREPQVYTLPPSQEEMTKNQVSLTCLVKGFYPSDIAVE | 161 |
| | | WESNGQPENNYKTTPPVLDSDGSFFLYSRLTVDKSRWQEGNVFSCSVMHE | |
| | | ALHNHYTQKSLSLSLGK | |
| EP560 | 2020EP47- | METDTLLLWVLLLWVPGSTGQVQLQQWGAGLLKPSETLSLTCAVYGGSFS | With |
| | F02_IgG_ | GYYWSWIRQPPGKGLEWIGEINHSGSTNYNPSLKSRVTISVDTSKNQFSL | Signal |
| | HC, | KLSSVTAADTAVYYCARGWYLGFDYWGQGTLVTVSSASTKGPSVFPLAPC | Peptide: |
| | anti- | SRSTSESTAALGCLVKDYFPEPVTVSWNSGALTSGVHTFPAVLQSSGLYS | 162 |
| | Nectin4 | LSSVVTVPSSSLGTKTYTCNVDHKPSNTKVDKRVESKYGPPCPPCPAPEF | Without |
| | | LGGPSVFLFPPKPKDTLMISRTPEVTCVVVDVSQEDPEVQFNWYVDGVEV | Signal |
| | | HNAKTKPREEQFNSTYRVVSVLTVLHQDWLNGKEYKCKVSNKGLPSSIEK | Peptide: |
| | | TISKAKGQPREPQVYTLPPSQEEMTKNQVSLTCLVKGFYPSDIAVEWESN | 163 |
| | | GQPENNYKTTPPVLDSDGSFFLYSRLTVDKSRWQEGNVFSCSVMHEALHN | |
| | | HYTQKSLSLSLGK | |
| EP695 | Single | EVQLVQSGAEVKKPGASVKVSCKASGYTFTGYYMHWVRQAPGQGLEWMGR | 164 |
| | point | INPNSGGTNYAQKFQGRVTMTRDTSISTAYMELSGLRSDDTAVYFCARVT | |
| | mutation | YNIGWYIDYWGQGTLVTVSSASTKGPSVFPLAPSSKSTSGGTAALGCLVK | |
| | within | DYFPEPVTVSWNSGALTSGVHTFPAVLQSSGLYSLSSVVTVPSSSLGTQT | |
| | HCDR2 of | YICNVNHKPSNTKVDKKVEPKSCDGGGGSGGGGSEVQLVESGGGLVQPGG | |
| | EP500 | SLRLSCAASGFTENTYAMNWVRQAPGKGLEWVARIRSKANNYATYYAASV | |
| | | KGRFTISRDDSKNSLFLQMNSLKTEDTAVYYCVRHGNFGNSYVSWFAYWG | |
| | | QGTLVTVSSGGGGSGGGGSGGGGSGGGGSQAVVTQEPSLTVSPGGTVTLT | |
| | | CASSTGAVTTSNYANWVQEKPDHLFTGLIGGTNKRAPGVPARFSGSLLGG | |
| | | KAALTLSGVQPEDEAEYYCALWYSNLWVFGGGTKLTVLEPKSCDKTHTCP | |
| | | PCPAPEAAGGPSVFLFPPKPKDTLMISRTPEVTCVVVDVSHEDPEVKFNW | |
| | | YVDGVEVHNAKTKPREEQYNSTYRVVSVLTVLHQDWLNGKEYKCKVSNKA | |
| | | LGAPIEKTISKAKGQPREPQVCTLPPSRDELTKNQVSLSCAVKGFYPSDI | |
| | | AVEWESNGQPENNYKTTPPVLDSDGSFKLVSKLTVDKSRWQQGNVFSCSV | |
| | | MHEALHNHYTQKSLSLSPGK | |
| EP696 | Two | EVQLVQSGAEVKKPGASVKVSCKASGYTFTGYYMHWVRQAPGQGLEWMGR | 165 |
| | residue | INPNSGGTNYAQKFQGRVTMTRDTSISTAYMELSGLRSDDTAVYFCARVT | |
| | mutations | YNIGWYIDYWGQGTLVTVSSASTKGPSVFPLAPSSKSTSGGTAALGCLVK | |
| | within | DYFPEPVTVSWNSGALTSGVHTFPAVLQSSGLYSLSSVVTVPSSSLGTQT | |
| | HCDR2 of | YICNVNHKPSNTKVDKKVEPKSCDGGGGSGGGGSEVQLVESGGGLVQPGG | |
| | EP500 | SLRLSCAASGFTFNTYAMNWVRQAPGKGLEWVARIRSKANAYATYYAASV | |
| | | KGRFTISRDDSKNSLFLQMNSLKTEDTAVYYCVRHGNFGNSYVSWFAYWG | |
| | | QGTLVTVSSGGGGSGGGGSGGGGSGGGGSQAVVTQEPSLTVSPGGTVTLT | |
| | | CASSTGAVTTSNYANWVQEKPDHLFTGLIGGINKRAPGVPARFSGSLLGG | |
| | | KAALTLSGVQPEDEAEYYCALWYSNLWVFGGGTKLTVLEPKSCDKTHTCP | |
| | | PCPAPEAAGGPSVFLFPPKPKDTLMISRTPEVTCVVVDVSHEDPEVKFNW | |
| | | YVDGVEVHNAKTKPREEQYNSTYRVVSVLTVLHQDWLNGKEYKCKVSNKA | |
| | | LGAPIEKTISKAKGQPREPQVCTLPPSRDELTKNQVSLSCAVKGFYPSDI | |
| | | AVEWESNGQPENNYKTTPPVLDSDGSFKLVSKLTVDKSRWQQGNVFSCSV | |
| | | MHEALHNHYTQKSLSLSPGK | |
| EP697 | Two | EVQLVQSGAEVKKPGASVKVSCKASGYTFTGYYMHWVRQAPGQGLEWMGR | 166 |
| | residue | INPNSGGTNYAQKFQGRVTMTRDTSISTAYMELSGLRSDDTAVYFCARVT | |
| | mutations | YNIGWYIDYWGQGTLVTVSSASTKGPSVFPLAPSSKSTSGGTAALGCLVK | |
| | within | DYFPEPVTVSWNSGALTSGVHTFPAVLQSSGLYSLSSVVTVPSSSLGTQT | |
| | HCDR2 and | YICNVNHKPSNTKVDKKVEPKSCDGGGGSGGGGSEVQLVESGGGLVQPGG | |
| | single point | SLRLSCAASGFTFNTYAMNWVRQAPGKGLEWVARIRSKANAYATYYAASV | |
| | mutation | KGRFTISRDDSKNSLFLQMNSLKTEDTAVYYCVRHGNFGNSAVSWFAYWG | |
| | with HCDR3 | QGTLVTVSSGGGGSGGGGSGGGGSGGGGSQAVVTQEPSLTVSPGGTVTLT | |
| | of EP500 | CASSTGAVTTSNYANWVQEKPDHLFTGLIGGINKRAPGVPARFSGSLLGG | |
| | | KAALTLSGVQPEDEAEYYCALWYSNLWVFGGGTKLTVLEPKSCDKTHTCP | |
| | | PCPAPEAAGGPSVFLFPPKPKDTLMISRTPEVTCVVVDVSHEDPEVKFNW | |
| | | YVDGVEVHNAKTKPREEQYNSTYRVVSVLTVLHQDWLNGKEYKCKVSNKA | |
| | | LGAPIEKTISKAKGQPREPQVCTLPPSRDELTKNQVSLSCAVKGFYPSDI | |
| | | AVEWESNGQPENNYKTTPPVLDSDGSFKLVSKLTVDKSRWQQGNVFSCSV | |
| | | MHEALHNHYTQKSLSLSPGK | |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An isolated antibody that binds Nectin Cell Adhesion Molecule 4 (nectin-4), wherein the antibody binds the same epitope as a reference antibody or competes against the reference antibody from binding to nectin-4, and wherein the reference antibody is selected from the group consisting of 2020EP034-H09, 2020EP034-B09, 2020EP034-E01, 2020EP47-F02, 2021EP030-B10, 2021EP030-C11, 2021EP030-D06, 2021EP030-E10, 2021EP030-F02, 2021EP030-H06, 2021EP029-C04, 2021EP032-D10, and 2021EP032-E06.

2. The isolated antibody of claim 1, wherein the antibody comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody.

3. The isolated antibody of claim 1, wherein the HC CDRs of the antibody collectively contain no more than 8 amino acid residue variations as compared with the HC CDRs of the reference antibody; and/or wherein the LC CDRs of the antibody collectively contain no more than 8 amino acid residue variations as compared with the LC CDRs of the reference antibody.

4. The isolated antibody of claim 1, wherein the antibody comprises a V_{H} that is at least 85% identical to the V_{H} of the reference antibody, and/or a V_{L} that is at least 85% identical to the V_{L} of the reference antibody.

5. The isolated antibody of claim 1, wherein the antibody has a binding affinity of less than about 25 nM to nectin-4 expressed on cell surface, optionally wherein the binding affinity is less than 10 nM, and preferably wherein the binding affinity is less than 1 nM.

6. The isolated antibody of claim 1, which comprises the same heavy chain complementary determining regions (HC CDRs) and the same light chain complementary determining regions (LC CDRs) as the reference antibody.

7. The isolated antibody of claim 6, which comprises the same V_{H} and the same V_{L} as the reference antibody.

8. The isolated antibody of claim 1, wherein the antibody is a human antibody or a humanized antibody.

9. The isolated antibody of claim 1, wherein the antibody is a single-chain antibody (scFv).

10. The isolated antibody of claim 1, wherein the antibody is a multi-chain molecule comprising at least two polypeptides.

11. The isolated antibody of claim 10, wherein each of the at least two polypeptides comprise an Fc fragment.

12. A multi-specific antibody, comprising:
a first binding moiety that binds nectin-4; and
a second binding moiety that binds CD3.

13. The multi-specific antibody of claim 12, wherein the first binding moiety comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody; and
wherein the reference antibody is selected from the group consisting of 2020EP034-H09, 2020EP034-B09, 2020EP034-E01, 2020EP47-F02, 2021EP030-B10, 2021EP030-C11, 2021EP030-D06, 2021EP030-E10, 2021EP030-F02, 2021EP030-H06, 2021EP029-C04, 2021EP032-D10, and 2021EP032-E06.

14. The multi-specific antibody of claim 12, wherein the first binding moiety and/or the second binding moiety is in single-chain variable fragment (scFv) format.

15. The multi-specific antibody of claim 12, wherein the first binding moiety and/or the second binding moiety is in immunoglobulin (Ig) format.

16. The multi-specific antibody of claim 12, wherein one of the first binding moiety and the second binding moiety is in scFv format and the other binding moiety is in Ig format.

17. The multi-specific antibody of claim 12, wherein:
(i) the first binding moiety comprises a first heavy chain and a first light chain, wherein the first heavy chain comprises a first heavy chain variable region (V_{H}) and a first heavy chain constant region, which comprises a first Fc fragment; and wherein the first light chain comprises a first light chain variable region (V_{L}) and a first light chain constant region; and
(ii) the second binding moiety comprises a second heavy chain and a second light chain, wherein the second heavy chain comprises a second heavy chain variable region (V_{H}) and a second heavy chain constant region, which comprises a second Fc fragment; and wherein the second light chain comprises a second light chain variable region (V_{L}) and a second light chain constant region; and
wherein the first Fc fragment and the second Fc fragment form a dimer.

18. The multi-specific antibody of claim 12, wherein:
(i) the first binding moiety comprises a first heavy chain, a second heavy chain, and a light chain, wherein the first heavy chain comprises V_{H} and a first heavy chain constant region, which comprises a first Fc fragment, wherein the second heavy chain comprises the V_{H} and a second heavy chain constant region, which comprises a second Fc fragment, and wherein the light chain comprises a V_{L} and a light chain constant region; and
(ii) the second binding moiety is an scFv fragment, which is fused with either the first heavy chain or the second heavy chain of (i), optionally wherein the scFv fragment is fused with the first or second heavy chain between the first or second Fc fragment and the V_{H}; and
wherein the first Fc fragment and the second Fc fragment form a dimer.

19. The multi-specific antibody of claim 12, wherein:
(i) the first binding moiety comprises a first heavy chain, a second heavy chain, and a light chain, wherein the first heavy chain comprises V_{H} and a first heavy chain constant region, which comprises a first Fc fragment, and wherein the second heavy chain comprises the V_{H} and a second heavy chain constant region, which comprises a second Fc fragment, and wherein the light chain comprises a V_{L} and a light chain constant region; and
(ii) the second binding moiety is a heavy chain only fragment (VHH), which is fused with either the first heavy chain or the second heavy chain of (i), optionally wherein the VHH fragment is fused with the first or second heavy chain between the first or second Fc fragment and the V_{H}; and
wherein the first Fc fragment and the second Fc fragment form a dimer.

20. The multi-specific antibody of claim 12, wherein:
(i) the first binding moiety comprises a first heavy chain and a first light chain, wherein the first heavy chain comprises a first heavy chain variable region (V_{H}) and a first heavy chain constant region, which comprises a first Fc fragment; and wherein the first light chain comprises a first light chain variable region (V_{L}) and a first light chain constant region; and
(ii) the second binding moiety is an scFv fragment fused to a second Fc fragment; and
wherein the first Fc fragment and the second Fc fragment form a dimer.

21. The multi-specific antibody of claim 17, wherein the first Fc fragment and the second Fc fragment comprise mutations that enhances heterodimeration over homodimeration as relative to the wild-type counterpart.

22. The multi-specific antibody of claim 21, wherein the mutations are knob-hole mutations.

23. The multi-specific antibody of claim 22, wherein the knob mutation is selected from the group consisting of S354C, T366W and K409A; and wherein the hole mutation is selected from the group consisting of S354C, Y349C, T366S, L368A, F405K, and Y407V.

24. The multi-specific antibody of claim 12, further comprises a cytokine, which optionally is IL-2.

25. The multi-specific antibody of claim 24, wherein the cytokine is fused to the C-terminus of the first Fc fragment, the C-terminus of the second Fc fragment, or both.

26. The multi-specific antibody of claim 12, further comprising a third binding moiety, which binds a T cell co-stimulatory receptor.

27. The multi-chain antibody of claim 26, wherein the T cell co-stimulatory receptor is ICOS, 4-1BB, CD28, or CD86.

28. A protein complex, comprising a first moiety that binds nectin-4 and a second moiety that comprises a cytokine.

29. The protein complex of claim 28, wherein the first moiety that binds nectin-4 comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody; and
wherein the reference antibody is selected from the group consisting of 2020EP034-H09, 2020EP034-B09, 2020EP034-E01, 2020EP47-F02, 2021EP030-B10, 2021EP030-C11, 2021EP030-D06, 2021EP030-E10, 2021EP030-F02, 2021EP030-H06, 2021EP029-C04, 2021EP032-D10, and 2021EP032-E06.

30. The protein complex of claim 28, wherein:
(i) the first moiety comprises an scFv fragment fused to a first Fc fragment; and
(ii) the second moiety comprises the cytokine fused to a second Fc fragment; and
wherein the first Fc fragment and the second Fc fragment form a dimer.

31. The protein complex of claim 28, wherein the first moiety comprises a first polypeptide, which comprises an scFv fragment fused to a first Fc fragment, and a second polypeptide, which comprises the scFv fragment fused to a second Fc fragment;
wherein the cytokine of the second moiety is fused to the C-terminus of the first Fc fragment, the C-terminus of the second Fc fragment, or both; and
wherein the first Fc fragment and the second Fc fragment form a dimer.

32. The protein complex of claim 28, wherein:
(i) the first moiety comprises a heavy chain comprising a V_{H} and a heavy chain constant region, which comprises a first Fc fragment, and a light chain comprising a V_{L} and a light chain constant region; and
(ii) the second moiety comprises the cytokine fused to a second Fc fragment;
wherein the first Fc fragment and the second Fc fragment form a dimer.

33. The protein complex of claim 28, wherein the first moiety comprises a first heavy chain comprising a V_{H} and a first heavy chain constant region, which comprises a first Fc fragment, a second heavy chain comprising the V_{H} and a second heavy chain constant region, which comprises a second Fc fragment, and a light chain comprising a V_{L} and a light chain constant region;
wherein the cytokine of the second moiety is fused to the C-terminus of the first Fc fragment, the C-terminus of the second Fc fragment, or both; and
wherein the first Fc fragment and the second Fc fragment form a dimer.

34. The protein complex of claim 28, wherein the cytokine is IL-2.

35. The protein complex of claim 28, wherein the first Fc fragment and the second Fc fragment comprise mutations that enhances heterodimeration over homodimeration as relative to the wild-type counterpart.

36. The protein complex of claim 35, wherein the mutations are knob-hole mutations.

37. The protein of claim 36, wherein the knob mutation is selected from the group consisting of S354C, T366W and K409A; and wherein the hole mutation is selected from the group consisting of S354C, Y349C, T366S, L368A, F405K, and Y407V.

38. A nucleic acid or a set of nucleic acids, which collectively encodes an antibody that binds Nectin Cell Adhesion Molecule 4 (nectin-4) or a multi-specific antibody comprising the antibody; wherein the antibody comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody; and
wherein the reference antibody is selected from the group consisting of 2020EP034-H09, 2020EP034-B09, 2020EP034-E01, 2020EP47-F02, 2021EP030-B10, 2021EP030-C11, 2021EP030-D06, 2021EP030-E10, 2021EP030-F02, 2021EP030-H06, 2021EP029-C04, 2021EP032-D10, and 2021EP032-E06.

39. The nucleic acid or the set of nucleic acids of claim 38, which is a vector or a set of vectors.

40. The nucleic acid or the set of nucleic acids or claim 39, wherein the vector is an expression vector.

41. A host cell comprising the nucleic acid or the set of nucleic acids of claim 38.

42. A pharmaceutical composition comprising an antibody that binds Nectin Cell Adhesion Molecule 4 (nectin-4) a multi-specific antibody or a protein complex comprising the antibody, or a nucleic acid or nucleic acids encoding the antibody, and a pharmaceutically acceptable carrier;
wherein the antibody comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody; and
wherein the reference antibody is selected from the group consisting of 2020EP034-H09, 2020EP034-B09, 2020EP034-E01, 2020EP47-F02, 2021EP030-B10, 2021EP030-C11, 2021EP030-D06, 2021EP030-E10, 2021EP030-F02, 2021EP030-H06, 2021EP029-C04, 2021EP032-D10, and 2021EP032-E06.

43. A method for inhibiting nectin-4 or nectin-4⁺ cells in a subject, comprising administering to a subject in need thereof any effective amount of the pharmaceutical composition of claim 42.

44. The method of claim 43, wherein the subject is a human patient having nectin-4⁺ pathogenic cells.

45. The method of claim 44, wherein the subject is a human patient having nectin-4 positive cancer, optionally wherein the cancer is breast cancer, bladder cancer, ovary cancer, cervical cancer, pancreatic cancer, lung cancer, or head and neck cancer.

46. A method for detecting presence of nectin, comprising:
(i) contacting an antibody of an antibody that binds Nectin Cell Adhesion Molecule 4 (nectin-4) with a sample suspected of containing nectin-4, and
(ii) detecting binding of the antibody to nectin-4; wherein the antibody comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody; and
wherein the reference antibody is selected from the group consisting of 2020EP034-H09, 2020EP034-B09, 2020EP034-E01, 2020EP47-F02, 2021EP030-B10, 2021EP030-C11, 2021EP030-D06, 2021EP030-E10, 2021EP030-F02, 2021EP030-H06, 2021EP029-C04, 2021EP032-D10, and 2021EP032-E06.

47. The method of claim 46, wherein the antibody is conjugated to a detectable label.

48. The method of claim 47, wherein the nectin-4 is expressed on cell surface.

49. The method of claim 48, wherein the contacting step is performed by administering the antibody to a subject.

50. A method of producing an antibody binding to nectin-4 or a multi-specific antibody or protein complex comprising such, comprising:
(i) culturing the host cell of claim 41 under conditions allowing for expression of the antibody that binds nectin-4, the multi-specific antibody comprising such, or the protein complex comprising such; and
(ii) harvesting the antibody, the multi-specific antibody, or the protein complex thus produced from the cell culture.
